SEQUENCE LISTING (46 sequences)

SEQ ID NO: 1 - DNA, 800 bp, Artificial Sequence
  Synthetic Sequence
acactaaatgcatgtttagattaaagcttgtaaatttaagtttgagttaaatttaagttagtaaattgagtttgcaaaaataacttagtgtactacgaaattgagtagttttcagacaaaattttagttgcacatagttttgaggataaccaaactatgtttagcattcaaaagtactttttgaacagttaaccaaacatgtcctaaatcattatacattaacaaattcctttattaaaaaaagtttaaatatgatttataatgttcatagtattaagttctggattattagtttttaaaacatttgatctataaggttagttttatcaagcggcaagtcaatcgtgtcgttcacatctttgcaagagcacctcgtttctatgctaattactgcattttttttattttaacctgtatgtatgatcttattttgaatgaaatgcaataagttatttctagtaaaaaaaaataaacatttgatagaaacaaattaaagcatgcaaaaataactcattagcatcggttaaattgaagggtttgaataatttgcacaaggttctgaattcaaatcttgttcattgtaaaaaataaagcatgaaaaaaagaggggcaaaatttaaacataaataataaggattcggtaagatcgagaatcgcaatgtagggattcagataaaaatatgttaagcagattgaaggataatatatatatatatatatatatatatatatatatatattgtatctgaaggataatattttaaatttactgaagcatagctccaaaattacgcggtttc

SEQ ID NO: 2 - DNA, 800 bp, Artificial Sequence
  Synthetic Sequence
ttgaagatttaggaacttggggtttatggaaattggaattgggattaagggtttgtatcccttgtgccatgttgttaatttgtgccattcttgaaagatctgctagagtcagcttgtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccatttgggttccctatgtttattttaacctgtatgtatgatcttattttgaatgaaatgcaataagttatttctagtaaaaaaaaataaacatttgatagaaacaaattaaagcatgcaaaaataactcattagcatcggttaaattgaagggtttgaataatttgcacaaggttctgaattcaaatcttgttcattgtaaaaaataaagcatgaaaaaaagaggggcaaaatttaaacataaataataaggattcggtaagatcgagaatcgcaatgtagggattcagataaaaatatgttaagcagattgaaggataatatatatatatatatatatatatatatatatatatattgtatctgaaggataatattttaaatttactgaagcatagctccaaaattacgcggtttc

SEQ ID NO: 3 - DNA, 25 bp, Artificial Sequence
  Synthetic Sequence
cagttaaccaaacatgtcctaaatc

SEQ ID NO: 4 - DNA, 21 bp, Artificial Sequence
  Synthetic Sequence
catcttcaacgatggcctttc

SEQ ID NO: 5 - DNA, 22 bp, Artificial Sequence
  Synthetic Sequence
tcaaacccttcaatttaaccga

SEQ ID NO: 6 - DNA, 800 bp, Artificial Sequence
  Synthetic Sequence
aagtccccatagattacataaccgacaaaaacaatgcccatatctaggaagccaatacagtcgatataaataacattaatccacacctaaatgtcataactcataaacaaccctaagcattaaattggagtccaagtactagagaaaggcttaatttcgtattgtaatctccctcagaatttcttaatcttgtgatcaacaaagcatatcctcgttttaaattctaaaggttatggcaaaattcactggcatacgaacaattcatatatccattcctattatatatagttggcagaagtacaaggaggcgccaaatagaaaacacaaattggaacggtgaagagaaagaagagtacctcggagagagttgaggcgagagatgagatcgggagggaagagattgggatcggagaagaactgtttgaggcgaatggcctggtcgtcgcggccatcgtcgagaagttcgtgaagaagctcgaatgcggtgagaaggtagttctcttccaacagaaagttcaccacgcaattgcacagcgaagatctctccacgtccattttctctctctgtctctgatcttaagccattcattcaagacaagacaagagaagagaagagaagagaagagaacactctcagtcagatcgtggtttcaactttcaagactgtgctagctagttaggtgccatcttacatgtttactttttttctttataagattaaattgctgaataccatgctctcctgtgtccaaagcagtacacccgcgtaaaaatagatttcatcgtcctttcgattttac

SEQ ID NO: 7 - DNA, 802 bp, Artificial Sequence
  Synthetic Sequence
aattaaataaatcaattacttcataaataattttttttatagaatatgttgacattctagccggatatagaactaatgtaaagaaaccttaaaaattttgtttggaagaatatgttattgaaagacaaatctaattaagtttatcagggtcatttgttgaagataggaaaccttcagcaatttgaatattaagtaactgcttctcccagaatgatcggagtttctcctcctgctattacatgagcaaaaataaaaaataaataaaagataagattaagcttcaacatgtgaaggagtagtacactcaccagtgaccctaataggcaacagcatgaaaaaaaataaaaaagaataaaaatagcatctacatatagcttctcgttgttagaaaaacaaaactatttgggatcggagaagaactgtttgaggcgaatggcctggtcgtcgcggccatcgtcgagaagttcgtgaagaagctcgaatgcggtgagaaggtagttctcttccaacagaaagttcaccacgcaattgcacagcgaagatctctccacgtccattttctctctctgtctctgatcttaagccattcattcaagacaagacaagagaagagaagagaagagaagagaacactctcagtcagatcgtggtttcaactttcaagactgtgctagctagttaggtgccatcttacatgtttactttttttctttataagattaaattgctgaataccatgctctcctgtgtccaaagcagtacacccgcgtaaaaatagatttcatcgtcctttcgattttac

SEQ ID NO: 8 - DNA, 22 bp, Artificial Sequence
  Synthetic Sequence
aagaagagtacctcggagagag

SEQ ID NO: 9 - DNA, 22 bp, Artificial Sequence
  Synthetic Sequence
ctcccagaatgatcggagtttc

SEQ ID NO: 10 - DNA, 21 bp, Artificial Sequence
  Synthetic Sequence
aactaccttctcaccgcattc

SEQ ID NO: 11 - DNA, 22 bp, Artificial Sequence
  Synthetic Sequence
cgagcttcttcacgaacttctc

SEQ ID NO: 12 - DNA, 18 bp, Artificial Sequence
  Synthetic Sequence
accgcattcgagcttctt

SEQ ID NO: 13 - DNA, 24 bp, Artificial Sequence
  Synthetic Sequence
ctttctgttggaagagaactacct

SEQ ID NO: 14 - DNA, 20 bp, Artificial Sequence
  Synthetic Sequence
gagagatcttcgctgtgcaa

SEQ ID NO: 15 - DNA, 18 bp, Artificial Sequence
  Synthetic Sequence
gcaattgcgtggtgaact

SEQ ID NO: 16 - DNA, 17 bp, Artificial Sequence
  Synthetic Sequence
aggccattcgcctcaaa

SEQ ID NO: 17 - DNA, 20 bp, Artificial Sequence
  Synthetic Sequence
cacgaacttctcgacgatgg

SEQ ID NO: 18 - DNA, 19 bp, Artificial Sequence
  Synthetic Sequence
ggccattcgcctcaaacag

SEQ ID NO: 19 - DNA, 24 bp, Artificial Sequence
  Synthetic Sequence
cccttcaatttaaccgatgctaat

SEQ ID NO: 20 - DNA, 23 bp, Artificial Sequence
  Synthetic Sequence
gcccatatctaggaagccaatac

SEQ ID NO: 21 - DNA, 28 bp, Artificial Sequence
  Synthetic Sequence
ccacacctaaatgtcataactcataaac

SEQ ID NO: 22 - DNA, 20 bp, Artificial Sequence
  Synthetic Sequence
agatcgggagggaagagatt

SEQ ID NO: 23 - DNA, 20 bp, Artificial Sequence
  Synthetic Sequence
gtacaaggaggcgccaaata

SEQ ID NO: 24 - DNA, 25 bp, Artificial Sequence
  Synthetic Sequence
ttcgtattgtaatctccctcagaat

SEQ ID NO: 25 - DNA, 25 bp, Artificial Sequence
  Synthetic Sequence
tccaagtactagagaaaggcttaat

SEQ ID NO: 26 - DNA, 24 bp, Artificial Sequence
  Synthetic Sequence
aggaagccaatacagtcgatataa

SEQ ID NO: 27 - DNA, 22 bp, Artificial Sequence
  Synthetic Sequence
tcactggcatacgaacaattca

SEQ ID NO: 28 - DNA, 24 bp, Artificial Sequence
  Synthetic Sequence
tggagtccaagtactagagaaagg

SEQ ID NO: 29 - DNA, 25 bp, Artificial Sequence
  Synthetic Sequence
tccctcagaatttcttaatcttgtg

SEQ ID NO: 30 - DNA, 25 bp, Artificial Sequence
  Synthetic Sequence
gaacagttaaccaaacatgtcctaa

SEQ ID NO: 31 - DNA, 24 bp, Artificial Sequence
  Synthetic Sequence
ttcgtattgtaatctccctcagaa

SEQ ID NO: 32 - DNA, 23 bp, Artificial Sequence
  Synthetic Sequence
ggagtttctcctcctgctattac

SEQ ID NO: 33 - DNA, 22 bp, Artificial Sequence
  Synthetic Sequence
acactcaccagtgaccctaata

SEQ ID NO: 34 - DNA, 20 bp, Artificial Sequence
  Synthetic Sequence
tgatcggagtttctcctcct

SEQ ID NO: 35 - DNA, 25 bp, Artificial Sequence
  Synthetic Sequence
ggtcatttgttgaagataggaaacc

SEQ ID NO: 36 - DNA, 22 bp, Artificial Sequence
  Synthetic Sequence
aaggagtagtacactcaccagt

SEQ ID NO: 37 - DNA, 22 bp, Artificial Sequence
  Synthetic Sequence
cctaataggcaacagcatgaaa

SEQ ID NO: 38 - DNA, 23 bp, Artificial Sequence
  Synthetic Sequence
tcaacatgtgaaggagtagtaca

SEQ ID NO: 39 - DNA, 25 bp, Artificial Sequence
  Synthetic Sequence
gcatctacatatagcttctcgttgt

SEQ ID NO: 40 - DNA, 24 bp, Artificial Sequence
  Synthetic Sequence
gtacactcaccagtgaccctaata

SEQ ID NO: 41 - DNA, 22 bp, Artificial Sequence
  Synthetic Sequence
ccctaataggcaacagcatgaa

SEQ ID NO: 42 - DNA, 22 bp, Artificial Sequence
  Synthetic Sequence
caacgatggcctttcctttatc

SEQ ID NO: 43 - DNA, 802 bp, Artificial Sequence; identical to SEQ ID NO: 7
  Synthetic Sequence

SEQ ID NO: 44 - DNA, 800 bp, Artificial Sequence
  Synthetic Sequence
gaaaccgcgtaattttggagctatgcttcagtaaatttaaaatattatccttcagatacaatatatatatatatatatatatatatatatatatatattatccttcaatctgcttaacatatttttatctgaatccctacattgcgattctcgatcttaccgaatccttattatttatgtttaaattttgcccctctttttttcatgctttattttttacaatgaacaagatttgaattcagaaccttgtgcaaattattcaaacccttcaatttaaccgatgctaatgagttatttttgcatgctttaatttgtttctatcaaatgtttatttttttttactagaaataacttattgcatttcattcaaaataagatcatacatacaggttaaaataaacatagggaacccaaatggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctttcaagaatggcacaaattaacaacatggcacaagggatacaaacccttaatcccaattccaatttccataaaccccaagttcctaaatcttcaa

SEQ ID NO: 45 - DNA, 800 bp, Artificial Sequence
  Synthetic Sequence
  misc_feature (118)..(118): n is c or g
  misc_feature (211)..(211): n is c or g
  misc_feature (219)..(219): n is a or c
  misc_feature (564)..(564): n is t or c
  misc_feature (596)..(596): n is c or g
  misc_feature (601)..(601): n is c or g
  misc_feature (611)..(611): n is g or c
  misc_feature (619)..(619): n is g or c
aagtccccatagattacataaccgacaaaaacaatgcccatatctaggaagccaatacagtcgatataaataacattaatccacacctaaatgtcataactcataaacaaccctaagnattaaattggagtccaagtactagagaaaggcttaatttcgtattgtaatctccctcagaatttcttaatcttgtgatcaacaaagcatatcntcgttttnaattctaaaggttatggcaaaattcactggcatacgaacaattcatatatccattcctattatatatagttggcagaagtacaaggaggcgccaaatagaaaacacaaattggaacggtgaagagaaagaagagtacctcggagagagttgaggcgagagatgagatcgggagggaagagattgggatcggagaagaactgtttgaggcgaatggcctggtcgtcgcggccatcgtcgagaagttcgtgaagaagctcgaatgcggtgagaaggtagttctcttccaacagaaagttcaccacgcaattgcacagcgaagatctctccacgtccattttctctcnctgtctctgatcttaagccattcattcaaganaaganaagagaaganaagagaanagaagagaacactctcagtcagatcgtggtttcaactttcaagactgtgctagctagttaggtgccatcttacatgtttactttttttctttataagattaaattgctgaataccatgctctcctgtgtccaaagcagtacacccgcgtaaaaatagatttcatcgtcctttcgattttac

SEQ ID NO: 46 - DNA, 800 bp, Artificial Sequence
  Synthetic Sequence
  misc_feature (2)..(2): n is A or G
  misc_feature (31)..(31): n is G or A
  misc_feature (131)..(131): n is G or A
  misc_feature (154)..(154): n is A or G
  misc_feature (160)..(160): n is C or T
  misc_feature (318)..(318): n is C or T
  misc_feature (331)..(332): n is A or T
  misc_feature (389)..(389): n is G or T
  misc_feature (397)..(398): n is T or A
  misc_feature (405)..(405): n is A, T, G, or C
  misc_feature (406)..(406): n is T or G
  misc_feature (538)..(538): n is A or C
  misc_feature (564)..(564): n is T or G
  misc_feature (615)..(615): n is G or A
  misc_feature (626)..(626): n is A or C
  misc_feature (694)..(694): n is T or A
gnaaccgcgtaattttggagctatgcttcantaaatttaaaatattatccttcagatacaatatatatatatatatatatatatatatatatatatattatccttcaatctgcttaacatatttttatctnaatccctacattgcgattctcgntcttancgaatccttattatttatgtttaaattttgcccctctttttttcatgctttattttttacaatgaacaagatttgaattcagaaccttgtgcaaattattcaaacccttcaatttaaccgatgctaatgagttatttttgcatgctttaatttgtttntatcaaatgtttnnttttttttactagaaataacttattgcatttcattcaaaataagatcatacatacangttaaaannaaaaaanngcagtaattagcatagaaacgaggtgctcttgcaaagatgtgaacgacacgattgacttgccgcttgataaaactaaccttatagatcaaatgttttaaaaactaataatccagaacttaatactatgaacnttataaatcatatttaaacttttttnaataaaggaatttgttaatgtataatgatttaggacatgtttggttaactnttcaaaaagtncttttgaatgctaaacatagtttggttatcctcaaaactatgtgcaactaaaattttgtctgaaaacnactcaatttcgtagtacactaagttatttttgcaaactcaatttactaacttaaatttaactcaaacttaaatttacaagctttaatctaaacatgcatttagtgt

(19) United States
(12) Patent Application Publication
(10) Pub. No.: US 2022/0177965 A1
(43) Pub. Date: Jun. 9, 2022

(54) RELATIVE QUANTIFICATION OF GENETIC VARIANTS IN A SAMPLE

(71) Applicant: Ontera Inc., Santa Cruz, CA (US)

(72) Inventors: Michael Pearson, Santa Cruz, CA (US); Trevor J. Morin, Santa Cruz, CA (US)

(21) Appl. No.: 17/437,028

(22) PCT Filed: Mar. 5, 2020
(86) PCT No.: PCT/US2020/021233

§ 371 Date: Sep. 7, 2021

Related U.S. Application Data
(60) Provisional application No. 62/815,763, filed on Mar. 8, 2019.
(60) Provisional application No. 62/933,935, filed on Nov. 11, 2019.

(51) Int. Cl. C12Q 1/6876; C12Q 1/6825; G01N 21/64; G01N 27/04

(52) U.S. Cl. CPC C12Q 1/6876; C12Q 1/6825; G01N 2021/6439; G01N 27/04; C12Q 2600/166; G01N 21/6428

(57) ABSTRACT

Provided herein is a method for determination of the frequency of a genetic rearrangement within the combined DNA from a population, and for determination of the fraction or amount of any physical or chemical property correlated with a genetic rearrangement in a population.

### CROSS-REFERENCE TO RELATED APPLICATIONS

This application claims the benefit of U.S. Provisional Patent Application No. 62/815,763, filed Mar. 8, 2019, and U.S. Provisional Patent Application No. 62/933,935, filed Nov. 11, 2019, each of which applications is incorporated herein by reference in its entirety.

### INCORPORATION BY REFERENCE OF SEQUENCE LISTING PROVIDED AS A TEXT FILE

A Sequence Listing is provided herewith as a text file, “45641 WO Seq Listing ST25.txt” created on Mar. 4, 2020 and having a size of 19 KB. The contents of the text file are incorporated by reference herein in their entirety.

### INTRODUCTION

Genetic rearrangements, such as translocations, inversions, duplications, deletions, and insertions in the genomes of organisms (including cells and viruses) can be benign, or they can lead to phenotypic changes. Some rearrangements lead to significant phenotypic alterations, especially when located within a functional gene, or when the insertion is a transgene that was artificially introduced to alter the phenotype of the organism.

Determination of the presence, or absence, of a specific known genetic rearrangement in a single organism is simple, and can be determined using endpoint PCR, microarrays, DNA sequencing, in-situ hybridization of probes, and other methods. In a haploid or diploid organism, the zygosity can be determined by performing a set of binary tests, each testing for the presence, or the absence of each possible variant. The final result is the exact state of the alleles present in the organism.

For some purposes, however, the frequency of a known genetic rearrangement in a large population of organisms (or in the combined DNA from a population of many organisms) is more important than the genetic state of any particular organism in that population. Determination of this frequency is a much more difficult problem.

For some other purposes, the fraction or amount of a physical or chemical property correlated with a genetic rearrangement in a large population of organisms is more important than the physical or chemical property of any particular organism in that population. For example, we may know that the mass of a portion of the population is highly correlated to the frequency of a genetic rearrangement in the pooled DNA from the population. Determination of this value is also a difficult problem.

The most straightforward method to determine the frequency of a genetic rearrangement in a population is through the individual testing of zygosity in either all individuals in the population, or a statistically relevant number of individuals. If a physical or chemical property of each individual was also tested, then the correlation between the property and the genotype can be calculated. In this way, the frequency of the rearrangement in a new test population can be used to calculate the correlated physical or chemical property in the test population. Although accurate, this is not practical for all applications, due to the time and cost involved.

An alternative approach is to perform a single test on a homogenized sample generated from either all individuals in the population, or a statistically relevant number of individuals. The state-of-the-art for this type of test relies on the use of qPCR or digital PCR technology. Using these technologies, the level of a genetic rearrangement is typically determined relative to the level of an independent reference gene. The reference gene is selected to be a highly conserved gene, present at a level of 100% in the same population sample as the rearrangement of interest.

Using qPCR, the levels of the rearrangement of interest and the reference gene are determined by a monitoring a fluorescence signal that is directly correlated to the rate of amplification during the PCR. The number of cycles that it takes for a fluorescence signal to pass a certain threshold is used to determine how many copies were present at the beginning of the reaction. This method requires that both of the PCR amplifications are completely independent, both reactions have close to 100% efficiency, and the amplicons are close to the same length. Using more than one type of fluorescent probe, and an excess of common PCR components, allows for both of the completely independent PCRs to be multiplexed within the same reaction volumes, but the analysis is the same as if they had been in different reactions. The exact and/or relative lengths, mass, charge, or other non-fluorescent properties of the two DNA amplicons are not measured, and they are not important to the result. The exact and/or relative number of the two DNA amplicons at the end point of the PCR are not important to the result.

Using digital PCR, the test sample is diluted to the point where single DNA molecules are distributed into thousands of individual amplification reactions containing primers for the reference gene. In parallel, the same diluted sample is tested with primers for the rearrangement of interest. Primers for both reactions are chosen such that the PCR amplicons are short, and close to the same length. The completely independent PCRs proceed to an end-point, and a fluorescent signal indicates when any of the thousands of reaction volumes contained the DNA specified by the primers. The frequency is determined by comparing the number of reactions positive for the rearrangement to the number of reactions positive for the reference gene. Using more than one type of fluorescent probe allows for both of the completely independent PCRs to be multiplexed within the same reaction volumes, but the analysis is the same as if they had been in different reactions. The exact and/or relative lengths, mass, charge, or other non-fluorescent properties of the two DNA amplicons are not measured. The exact and/or relative number of the two DNA amplicons are not measured, and they are not important to the result.

What is needed, therefore, are improved methods to determine the frequency of a genetic variant in a population.

### SUMMARY

Aspects of the present disclosure include methods of an amplification reaction and analysis to determine the frequency of a two genetic sequences in a sample. Aspects of the present disclosure include methods of quantifying a relative amount of genetic variants in a sample. In some embodiments, the method comprises quantifying the frequency of two genetic variants in a mixed population of a plurality of genetically variable organisms (e.g. seeds), and analyzing the results based on the amount of two different length PCR products. In some embodiments, the reaction is made quanitative by limiting a common primer.

Provided herein is a method for determination of the frequency of a genetic rearrangement within the combined DNA from a population, and for determination of the fraction or amount of any physical or chemical property correlated with a genetic rearrangement in a population.

In some embodiments, the method of the present disclosure of quantifying a relative amount of genetic variants in a sample comprise mixing said sample with a set of primers capable of binding specifically to a target sequence to initiate an amplification reaction, said set of primers comprising: a first primer that binds specifically to a common sequence on a first strand of a first variant and a second variant in the sample, wherein said first primer is added at a reaction limiting concentration; a second primer that binds specifically to a second strand of said first variant; and a third primer that binds specifically to a second strand of said second variant; performing an amplification reaction on said mixed sample to generate two amplification products of different length, wherein said first amplification product is generated from the first and second primer, and wherein the second amplification product is generated from the first and third primer; detecting at least two distinct signals corresponding to the first amplification product and the second amplification product; and quantifying the relative amount of the first and the second amplification products based on said detected signals.

In some embodiments, the amplification reaction is limited to align amplification rates of said first and second variants.

In some embodiments, at least one component of the amplification reaction is provided at a limiting reaction to align amplification rates of said first and second variants.

In some embodiments, the amplification reaction is inhibited by PCR conditions, a PCR blocking oligonucleotide, or sequence specific cleavage of the DNA template.

In some embodiments, the sample is derived from an organism or a population of organisms.

In some embodiments, the relative amount of genetic variants is used to determine a zygosity of said organism.

In some embodiments, the organism is suspected of being a genetically modified organism.

In some embodiments, at least one of said genetic variants is recombinantly engineered.

In some embodiments, the method further comprises amplifying a control gene in said sample, and quantifying one or both of said amplification products relative to said amplified control gene.

In some embodiments, the quantification determines a zyogosity of an organism comprising said genetic variants.

In some embodiments, at least one of said genetic variants comprises a recombinantly engineered gene.

In some embodiments, least one of said genetic variants comprise an inserted sequence.

In some embodiments, at least one of said genetic variants comprises a genetic rearrangement.

In some embodiments, the sample is derived from a virus, a protozoan, a fungus, a mold, a plant, an animal, or a human.

In some embodiments, the amplification reaction is selected from PCR or isothermal amplification.

In some embodiments, the distinct signal is detected using a nanopore device.

In some embodiments, the signals from said first and second genetic variants are discriminated by a characteristic selected from the group consisting of: amplicon length, sequence, physical or chemical modification incorporated into the primer, and physical or chemical probe added to the amplicon post-amplification.

In some embodiments, the physical or chemical probe comprises PEG.

In some embodiments, the physical or chemical probe comprises a fluorophore

In some embodiments, the PEG or fluorophore is bound to DNA, LNA, XNA, or PNA.

In some embodiments, the amplification reaction comprises one or more modified nucleotides or one or more modified primers.

In some embodiments, the modification comprises a direct label or an indirect label.

In some embodiments, the modification comprises a charged chemical moiety, a neutral chemical moiety, a hydrophobic moiety, or a hydrophilic moiety.

In some embodiments, the modification comprises a fluorescent dye.

In some embodiments, the detection is performed using a sensor configured to measures an electrical signal that fluctuates upon translocation of said first and/or second amplification product through a nanopore.

In some embodiments, the electrical signal is distinct between said first and second amplification products.

In some embodiments, the set of primers further comprises a fourth primer and a fifth primer that each each bind to a third strand and a fourth strand, wherein the third primer binds to the third strand.

In some embodiments, the method comprises performing said amplification reaction on said mixed sample further generates a third amplification product and a fourth amplification product.

In some embodiments, the four amplification products are each of different lengths.

In some embodiments, the four amplification products are of three different lengths, with two amplification products being the same length.

In some embodiments, the third amplification product is generated from the fourth primer and the third primer, and said fourth amplification product is generated from the fourth primer and the fifth primer.

In some embodiments, the first or second variant comprises a single nucleotide polymorphism.

In some embodiments, the first or second variant comprises a silent mutation, a missense mutation, or a nonsense mutation.

In some embodiments, the first or second variant comprises a modified nucleotide or a non-natural nucleotide.

In some embodiments, the method further comprises, prior to detecting, loading the first amplification product onto a nanopore device.

In some embodiments, the method further comprises, prior to detecting, loading the second amplification product onto a nanopore device.

In some embodiments, the method further comprises applying a voltage at least one nanopore for translocating the first and/or second amplification product through the at least one nanopore.

In some embodiments, the first primer is a forward primer selected from

(SEQ ID NO: 5)
TCAAACCCTTCAATTTAACCGA;
(SEQ ID NO: 10)
AACTACCTTCTCACCGCATTC;
(SEQ ID NO: 11)
CGAGCTTCTTCACGAACTTCTC;
(SEQ ID NO: 12)
ACCGCATTCGAGCTTCTT;
(SEQ ID NO: 13)
CTTTCTGTTGGAAGAGAACTACCT;
(SEQ ID NO: 14)
GAGAGATCTTCGCTGTGCAA;
(SEQ ID NO: 15)
GCAATTGCGTGGTGAACT;
(SEQ ID NO: 16)
AGGCCATTCGCCTCAAA;
(SEQ ID NO: 17)
CACGAACTTCTCGACGATGG;
(SEQ ID NO: 18)
GGCCATTCGCCTCAAACAG;
and
(SEQ ID NO: 19)
CCCTTCAATTTAACCGATGCTAAT.

In some embodiments, the second primer is a reverse primer selected from:

(SEQ ID NO: 3)
CAGTTAACCAAACATGTCCTAAATC;
(SEQ ID NO: 20)
GCCCATATCTAGGAAGCCAATAC;
(SEQ ID NO: 8)
AAGAAGAGTACCTCGGAGAGAG;
(SEQ ID NO: 21)
CCACACCTAAATGTCATAACTCATAAAC;
(SEQ ID NO: 22)
AGATCGGGAGGGAAGAGATT;
(SEQ ID NO: 23)
GTACAAGGAGGCGCCAAATA;
(SEQ ID NO: 24)
TTCGTATTGTAATCTCCCTCAGAAT;
(SEQ ID NO: 25)
TCCAAGTACTAGAGAAAGGCTTAAT;
(SEQ ID NO: 26)
AGGAAGCCAATACAGTCGATATAA;
(SEQ ID NO: 27)
TCACTGGCATACGAACAATTCA;
(SEQ ID NO: 28)
TGGAGTCCAAGTACTAGAGAAAGG;
(SEQ ID NO: 29)
TCCCTCAGAATTTCTTAATCTTGTG;
(SEQ ID NO: 30)
GAACAGTTAACCAAACATGTCCTAA;
and
(SEQ ID NO: 31)
TTCGTATTGTAATCTCCCTCAGAA.

In some embodiments, the third primer is a reverse primer selected from

(SEQ ID NO: 4)
CATCTTCAACGATGGCCTTTC;
(SEQ ID NO: 32)
GGAGTTTCTCCTCCTGCTATTAC;
(SEQ ID NO: 9)
CTCCCAGAATGATCGGAGTTTC;
(SEQ ID NO: 33)
ACACTCACCAGTGACCCTAATA;
(SEQ ID NO: 34)
TGATCGGAGTTTCTCCTCCT;
(SEQ ID NO: 35)
GGTCATTTGTTGAAGATAGGAAACC;
(SEQ ID NO: 36)
AAGGAGTAGTACACTCACCAGT;
(SEQ ID NO: 37)
CCTAATAGGCAACAGCATGAAA;
(SEQ ID NO: 38)
TCAACATGTGAAGGAGTAGTACA;
(SEQ ID NO: 39)
GCATCTACATATAGCTTCTCGTTGT;
(SEQ ID NO: 40)
GTACACTCACCAGTGACCCTAATA,
(SEQ ID NO: 41)
CCCTAATAGGCAACAGCATGAA;
and
(SEQ ID NO: 42)
CAACGATGGCCTTTCCTTTATC.

### BRIEF DESCRIPTION OF THE DRAWINGS

FIG. 1 depicts the analysis after separation of PCR products—capillary electrophoresis with fluorescence.

FIG. 2 depicts a correction equation showing a plot of % weight (y-axis) vs % moleculesM (x-axis) and a curve was fit with the following equation: y=−0.0000002240812246x⁴+0.0000798384452526x³−0.0155731965304255x²+1.9814122167848600x.

FIG. 3 depicts a correction equation with a plot of % weight (y-axis) vs % fluorescenceM (x-axis) and a curve was fit with the following equation: y=−0.0000009572152848x⁴+0.0001704660333688x³−0.0095159536947165x²+1.1994295515901300x.

FIG. 4 depicts a correction equation with a a plot of % weightM (y-axis) vs % eventsM (x-axis) and a curve was fit with the following equation: y=0.0000015901859517x⁴−0.0003883825100108x³+0.0255081044479226x²+0.7447058163287470x.

FIG. 5 depicts a diagram of primer arrangements for quanitification of genetic variants, such as an insertion, where identification of distinct genetic variants can be obtained using features such as PCR product length.

FIG. 6 depicts depicts a diagram of primer arrangements for quanitification of genetic variants, such as an insertion, where identification of distinct genetic variants can be obtained using features such as PCR product length.

FIG. 7 depicts a diagram of primer arrangements for quanitification of genetic variants, such as an insertion, where identification of distinct genetic variants can be obtained using features such as modification of primers.

FIG. 8 depicts a diagram of primer arrangements for quanitification of genetic variants, such as an insertion, where identification of distinct genetic variants can be obtained using features such as sequence specific probes.

FIG. 9 depicts a diagram of primer arrangements for quanitification of genetic variants, such as an insertion, where identification of distinct genetic variants can be obtained using features such as probes that are altered during the reaction (e.g., fluorescent probe alteration).

FIG. 10 depicts a diagram of primer arrangements for quanitification of genetic variants, such as a deletion, where identification of distinct genetic variants can be obtained using features such as PCR product length.

FIG. 11 depicts a diagram of primer arrangements for quanitification of genetic variants, such as a deletion, where identification of distinct genetic variants can be obtained using features such as PCR product length.

FIG. 12 depicts a diagram of primer arrangements for quanitification of genetic variants, such as a deletion, where identification of distinct genetic variants can be obtained using features such as modification of primers.

FIG. 13 depicts a diagram of primer arrangements for quanitification of genetic variants, such as a deletion, where identification of distinct genetic variants can be obtained using features such as sequence specific probes.

FIG. 14 depicts a diagram of primer arrangements for quanitification of genetic variants, such as a deletion, where identification of distinct genetic variants can be obtained using features such as probes that are altered during the reaction (e.g., fluorescent probe alteration).

FIG. 15 depicts a diagram of primer arrangements for quanitification of genetic variants, such as translocation, where identification of distinct genetic variants can be obtained using features such as PCR product length.

FIG. 16 depicts a diagram of primer arrangements for quanitification of genetic variants, such as translocation, where identification of distinct genetic variants can be obtained using features such as PCR product length.

FIG. 17 depicts depicts a diagram of primer arrangements for quanitification of genetic variants, such as translocation, where identification of distinct genetic variants can be obtained using features such as modification of primers.

FIG. 18 depicts a diagram of primer arrangements for quanitification of genetic variants, such as translocation, where identification of distinct genetic variants can be obtained using features such as sequence specific probes.

FIG. 19 depicts a diagram of primer arrangements for quanitification of genetic variants, such as translocation, where identification of distinct genetic variants can be obtained using features such as probes that are altered during the reaction (e.g., fluorescent probe alteration).

FIG. 20 depicts an example quantification of specific genetic variants in a population using endpoint 3-primer PCR with different product lengths.

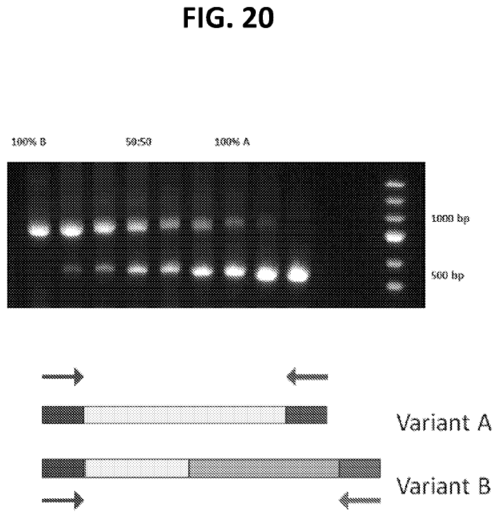

FIG. 21 shows an example of competitive PCR with conventional soy DNA and compared to the mutant variant indicating RR soybean DNA.

FIG. 22 shows an example of quantitive PCR with lectin and competitive PCR with insertion of conventional soybean DNA and a mutant variant indicating RR soybean DNA with resulting amplicons.

FIG. 23 depicts the percent GM trait vs amount of PCR products. (Top graph) Lectin is always amplified, independent of the soy DNA. It is used to count total soybeans amplified so it always equals 100%. When GMO DNA is high, it is harder to differentiated percentage of GMO present. (Bottom Graph) Only WT or GM DNA is amplified, not both. It is easy to see the percentage of GMO present even at high concentrations of GMO DNA.

FIG. 24 shows electrical current impedance measurements vs time spend in the nanopore of WT, and mutant variant RR soybean DNA using a nanopore device.

FIG. 25 shows a spectral graph of electrical current impedance measurements vs time of WT and mutant variant RR soybean DNA

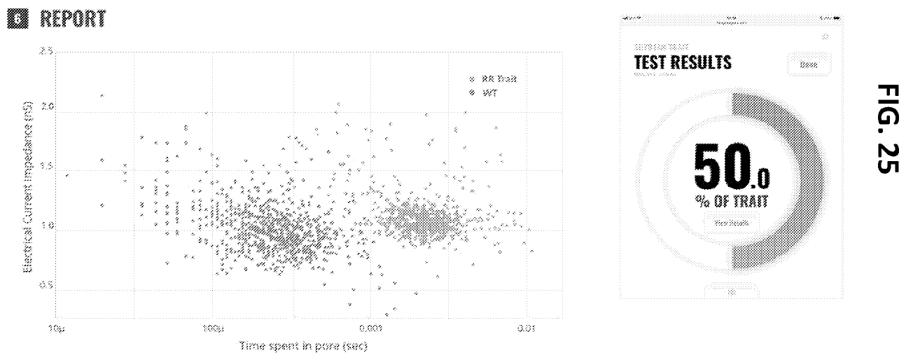

FIG. 26 depicts a specificity test for assay 2 qualitatively shows the expected ratios of Trait vs. Non-Trait amplicons. The % Trait-Extract PCR products are made from 0% Trait, 50% Trait, and 100% Trait seed mixes, with the exception of the 50% Mix which was made from Extracts (Step 3). The products are visualized using the Gel Electrophoresis Protocol, showing the Trait 298 bp and Non-trait 153 bp amplicon lengths. 6% TBE PAGE gel run at 200V for 25 minutes. Stained with SYBR Green for 15 minutes. Imaged using Bio-Rad ChemiDoc M

FIG. 27 depicts a table with PCR quantification of FIG. 26 data using a capillary electrophoresis protocol.

FIG. 28 depicts a table with the % trait PCR values produced using a capillary electrophoresis protocol for reference experiments A and B.

FIG. 29 depicts the correlation between the % Trait-Extract and % Trait PCR is used to fit a Calibration Equation. The 21 data points are the combined values from Experiments A and B in the table in FIG. 28. % Trait PCR values are plotted on the horizontal axis so the fit can convert % Trait PCR values, produced form analysis of unknown raw seed mixtures, into % Trait-Extract predictions.

FIG. 30 depicts a table with the % Trait PCR values produced using the capillary electrophoresis protocol for test experiment C.

FIG. 31 depicts a table with calculated % Trait-Extract values by applying the calibration equations to the test % Trait PCR data from the table in FIG. 30.

FIG. 32 depicts the % Trait PCR predictions generated by applying the support vector machine method to nanopore data and also the 2^{nd} degree calibration equation.

FIG. 33 depicts qualitative gels of sixteen assays tested with 0%, 50%, and 100% Trait-Extract. 6% TBE PAGE gel run at 200V for 25 minutes. Stained with SYBR Green for 15 minutes. Imaged using Bio-Rad ChemiDoc MP

FIG. 34 depicts the specificity test of assays 2, 14, and 16 with templates made from 0%, 50%, and 100% Trait Extract and Extract-Mixes. 6% TBE PAGE gel run at 200V for 25 minutes. Stained with SYBR Green for 15 minutes. Imaged using Bio-Rad ChemiDoc MP

FIG. 35 depicts qualitative gels of Experiments A (left) and B (right) for assays 12, 14, and 16. 6% TBE PAGE gel run at 200V for 25 minutes. Stained with SYBR Green for 15 minutes. Imaged using Bio-Rad ChemiDoc MP

FIG. 36 depicts qualitative gel of assay 2 experiment C. 6% TBE PAGE gel run at 200V for 25 minutes. Stained with SYBR Green for 15 minutes. Imaged using Bio-Rad ChemiDoc MP

FIG. 37 depicts qualitative gel of Assay 2 Experiments C1-C3 (C1 is the same as Experiment C in FIG. 36). 6% TBE PAGE gel run at 200V for 25 minutes. Stained with SYBR Green for 15 minutes. Imaged using Bio-Rad ChemiDoc MP

FIG. 38 depicts replicate lanes of triplicate PCR wells for fast PCR workflow.

FIG. 39 depicts qualitative gel of Assay 14 fast PCR samples (MBS device, PCR protocol B). 6% TBE PAGE gel run at 200V for 25 minutes. Stained with SYBR Green for 15 minutes. Imaged using Bio-Rad ChemiDoc MP.

FIG. 40 depicts a table with sixteen total assays with primers that were gel proofed (Suppl Doc S2, FIG. 33).

FIG. 41 depicts a table with Capillary electrophoresis protocol applied to 50% Trait mixtures for the sixteen different three-primer assays following end-point PCR.

FIG. 42 depicts a table with data and calculated Calibration Equations for assays 2, 14 and 16 using Experiment A and B data permutations.

FIG. 43 depicts a table with data and calculated Calibration Equations for assays 2, 14 and 16 using Experiment A and B data permutations.

FIG. 44 depicts a table with data and calculated Calibration Equations for assays 2, 14 and 16 using Experiment A and B data permutations.

FIG. 45 depicts a table with data and calculated Calibration Equations for assays 2, 14 and 16 using Experiment A and B data permutations.

FIG. 46 depicts a table with data and calculated Calibration Equations for assays 2, 14 and 16 using Experiment A and B data permutations.

FIG. 47 depicts a table with data and calculated Calibration Equations for assays 2, 14 and 16 using Experiment A and B data permutations.

FIG. 48 depicts a table with DNA sequences at the junctions of the transgenic insertion in Trait soybeans.

FIG. 49 depicts a table with triplicate repeats of experiment C data and analysis results. *Calibration equations are those found for Experiment A and B data, and reported in Example 3.

FIG. 50 depicts a table with Nanopore-based quantification results for of experiment C data. Raw Error Range is −7.14% to −0.09% 2nd Degree Calibration Error Range is −5.44% to 3.34%. *Calibration equations are from assay 2. Experiments A and B, with PCR Protocol A

FIG. 51 depicts depicts a table with Nanopore-based quantification results for of experiment C data. Raw Error Range is −6.33% to 0.79% 2nd Degree Calibration Error Range is −4.27% to 4.00%.

FIG. 52 depicts depicts nanopore sizes from experiment C as shown in Example 3 and in FIGS. 50-51. ***Diameter is starting value at the start of reagent testing, as described in “S1 Text”. A total of 16 chips were used, with 4 chips used for each of the Pore Set columns.

FIG. 53 depicts a table with Quantitative results of applying the capillary electrophoresis protocol to produce % Trait PCR values for the fast PCR products.

FIG. 54 depicts a table with Quantitative results of applying the capillary electrophoresis protocol to produce % Trait PCR values for the fast PCR products.

FIG. 55 depicts a table with Quantitative results of applying the capillary electrophoresis protocol to produce % Trait PCR values for the fast PCR products. **CI95% follows standard formula (A Concise Guide to Clinical Trials, Allan Hackshaw, pp. 205, 2009 ISBN: 978-1-405-16774-1) ***Table format patterened after reference: Huang, Chia-Chia, and Tzu-Ming Pan. “Event-Specific Real-Time Detection and Quantification of Genetically Modified Roundup Ready Soybean.” Journal of Agricultural and Food Chemistry 53, no. 10 (May 2005): 3833-39. doi:10.1021/jf048580x.

FIG. 56 illustrates a schematic of fabricated solid-state nanopore chip. The nanopore diameter is within 25-35 nm across an entire wafer.

FIG. 57 illustrates a schematic of the exploded and assembled views of the injection molded test strip. In the exploded view, the small square die between the molded gasket and bottom is the 3 mm×3 mm nanopore chip.

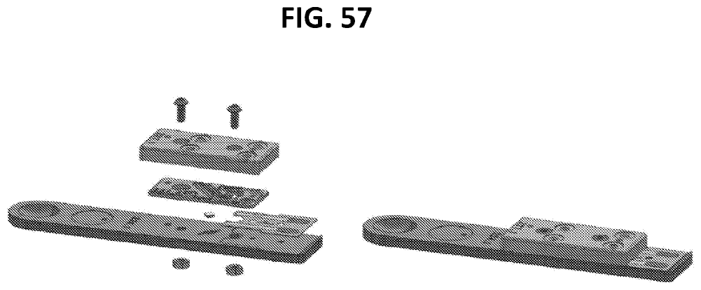

FIG. 58 depicts a table with the trained model used to classify the testing dataset events and scores the model's accuracy on unseen event data. The confusion matrix is also generated from the total of 575 events.

FIGS. 59A-59B depict nanopore event populations from (FIG. 59A) controls and (FIG. 59B) unknown mixture reagent runs, with model-based boundary for trait vs. non-trait event binning created in FIG. 59A and applied in FIG. 59B. Panel a, Superposition of events (max amplitude vs. base-10 log of dwell time duration) from 100% trait and 100% non-trait controls that were sequentially recorded, and the identified model-based grid boundary that is subsequently used for predictions. Panel b, Events from unknown reagents after binning each event using the model-based grid boundary in panel a). The true mixture is 30% trait, and the SVM prediction after applying equation (1) is 27.7%.

FIGS. 60A-60B depict a principle component analysis uses single 50% trait control mixture and then predicts trait % for the unknown mixture. FIG. 60A, The clustering result of the 50% mixture based on PCA, projected onto the one dimensional (1D) principal component (PC) axis that maximizes separation in event parameter space. FIG. 60B Events from the 50% mixture reagent that is shown after PCA in FIG. 60A. This is the same control mixture used for the SVM results in FIGS. 59A-59B.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

The term “target polynucleotide” used herein refers to a polynucleotide comprising a sequence of interest (i.e., a target polynucleotide sequence or a target sequence). A target polynucleotide can include regions (e.g., sufficiently complementary sequences) for hybridizing to primers for amplification of the target polynucleotide. These regions can be part of the sequence of interest, flanking the sequence of interest, or further upstream or downstream of the sequence of interest in sufficient proximity to allow amplification of the sequence of interest via an amplification reaction. In some embodiments, these regions for hybridizing to primers are located at the two ends of the amplicon generated by an amplification reaction. Described herein according to some embodiments are methods, devices, and compositions for detecting a target polynucleotide comprising a sequence of interest.

The term “amplification” or “amplification reaction” as used herein refers to a reaction that generates a plurality of clonal amplicons comprising a target polynucleotide sequence from the target polynucleotide sequence. As used herein, amplification reaction reagents include any molecules that are necessary to perform amplification of the target polynucleotide sequence. Amplification reaction reagents can include, but are not limited to, free primers, dNTPs (deoxynucleotide triphosphates, dATP, dGTP, dCTP, dTTP), polymerase enzymes (e.g., Taq or Pfu), salts (Magnesium chloride, Magnesium Sulfate, Ammonium sulfate, sodium chloride, potassium chloride), BSA (bovine serum albumin) stabilizer, and detergents (e.g., triton X-100). Amplification reactions can include, but are not limited to, e.g., PCR, ligase chain reaction (LCR), transcription mediated amplification (TMA), reverse transcriptase initiated PCR, DNA or RNA hybridization techniques, sequencing, isothermal amplification, and loop-mediated isothermal amplification (LAMP). Techniques of amplification to generate an amplicon from a target polynucleotide sequence are well known to one of skill in the art. In some embodiments, the method comprises combining the mixed sample with an effective amount of a buffer (e.g. HF buffer), DNA polymerase (Phusion hot start flex DNA polymerase, dNTPs, the set of primers, and water.

The term “nanopore” as used herein refers to an opening (hole or channel) of sufficient size to allow the passage of particularly sized polymers. With an amplifier, voltage is applied to drive negatively charged polymers through the nanopore, and the current through the pore detects if molecules are passing through it.

The term “sensor” as used herein refers to a device that collects a signal from a nanopore device. In many embodiments, the sensor includes a pair of electrodes placed at two sides of a pore to measure an ionic current across the pore when a molecule or other entity, in particular a polymer scaffold, moves through the pore. In addition to the electrodes, an additional sensor, e.g., an optical sensor, may be to detect an optical signal in the nanopore device. Other sensors may be used to detect such properties as current blockade, electron tunneling current, charge-induced field effect, nanopore transit time, optical signal, light scattering, and plasmon resonance.

The term “current measurement” as used herein refers to a series of measurements of current flow at an applied voltage through the nanopore over time. The current is expressed as a measurement to quantitate events, and the current normalized by voltage (conductance) is also used to quantitate events.

The term “open channel” as used herein refers to the baseline level of current through a nanopore channel within a noise range where the current does not deviate from a threshold of value defined by the analysis software.

The term “event” as used herein refers to a set of current impedance measurements that begins when the current measurement deviates from the open channel value by a defined threshold, and ends when the current returns to within a threshold of the open channel value.

The term “nanopore instrument” or “nanopore device” as used herein refers to a device that combines one or more nanopores (in parallel or in series) with circuitry for sensing single molecule events. Each nanopore within the nanopore device, including its chambers and/or channels and electrodes used to facilitate sensing with that nanopore, is referred to herein as a nanopore sensor. In some embodiments, nanopore instruments use a sensitive voltage-clamp amplifier to apply a specified voltage across the pore or pores while measuring the ionic current through the pore(s). When a single charged molecule such as a double-stranded DNA (dsDNA) or amplicon product is captured and driven through the pore by electrophoresis, the measured current shifts, indicating a capture event (i.e., the translocation of a molecule through the nanopore, or the capture of a molecule in the nanopore), and the shift amount (in current amplitude) and duration of the event are used to characterize the molecule captured in the nanopore. After recording many events during an experiment, distributions of the events are analyzed to characterize the corresponding molecule according to its shift amount (i.e., its current signature). In this way, nanopores provide a simple, label-free, purely electrical single-molecule method for biomolecular sensing.

The term “electrical signal” as used herein encompasses a series of data collected on current, impedance/resistance, or voltage over time depending on configuration of the electronic circuitry. Conventionally, current is measured in a “voltage clamp” configuration; voltage is measured in a “current clamp” configuration, and resistance measurements can be derived in either configuration using Ohm's law V=IR. Impedance can also be generated by measured from current or voltage data collected from the nanopore device. Types of electrical signals referenced herein include current signatures and current impedance signatures, although various other electrical signals may be used to detect particles in a nanopore.

### DETAILED DESCRIPTION

The details of various embodiments of the present disclosure are set forth in the description below. Other features, objects, and advantages of the invention will be apparent from the description and the drawings, and from the claims.

Aspects of the present disclosure include methods of an amplification reaction and analysis to determine the frequency of a two genetic sequences in a sample. Aspects of the present disclosure include methods of quantifying a relative amount of genetic variants in a sample. In some embodiments, the method comprises quantifying the frequency of two genetic variants in a mixed population of a plurality of genetically variable organisms (e.g. seeds), and analyzing the results based on the amount of two different length PCR products. In some embodiments, the reaction is made quantitative by limiting a common primer.

In some embodiments, provided herein is a method that uses a PCR with three primers, to determine the frequency of a specific genetic rearrangement within the combined DNA from a population of organisms. One common primer binds to a genomic site that is unaltered in all of the organisms. The other two primers are specific to genomic sites that differ in position due to the genetic rearrangement. During the PCR, the common primer will be used to amplify both of the amplicons, while the specific primers are only used to amplify one amplicon, or the other. The specific primers are designed to give the associated DNA amplicons differing measurable properties. In some embodiments, the common primer (e.g. first primer) is a forward primer.

In some embodiments, provided herein is a method that uses PCR with three primers to quantify the frequency of two genetic variants in a mixed population of a plurality of genetically variable organisms. In some cases, the organism comprises one or more, 10 or more, 100 or more, 500 or more, 1000 or more, 2000 or more, 5000 or more, or 10000 or more seeds. In some cases, the analysis is based on the amount of two different length PCR products. In some cases, the reaction is made quantitative by limiting the common primer.

Using the one common, and two specific primers, and a DNA template sample made from the combined DNA from a population of organisms, a PCR is performed. During the PCR, the three primers produce two DNA amplicons. At the end point of the PCR, the two DNA amplicons are present at a ratio that is correlated to the frequency of two alternative genetic rearrangement states present in the starting DNA template, and thus correlated to the frequency of phenotypic traits in the population that produced the DNA template.

In some embodiments, primers can bind to both variants, but cannot participate in the exponential amplification due to either PCR conditions (such as extention time), a PCR blocking oligonucleotide, a sequence specific cleavage of the DNA template by an enzyme, etc.)

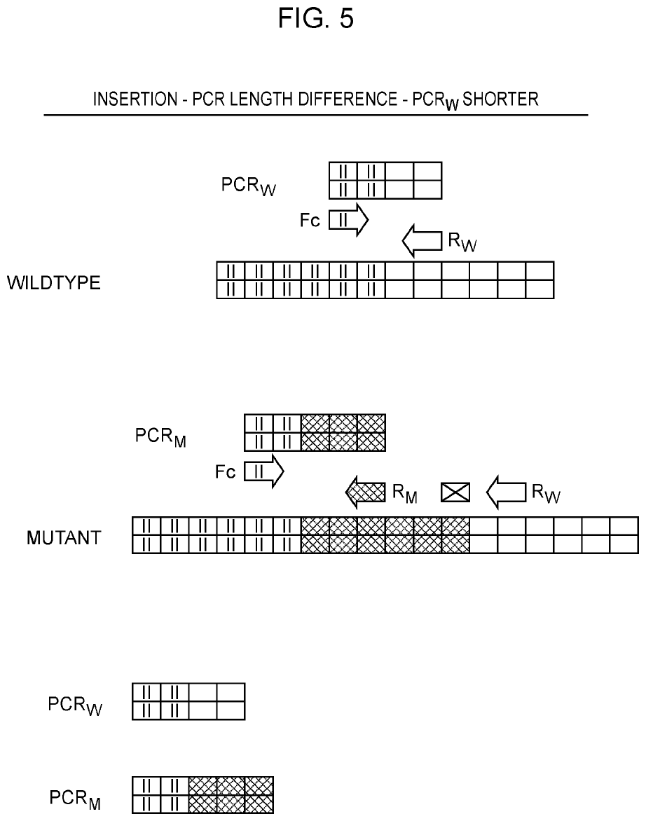

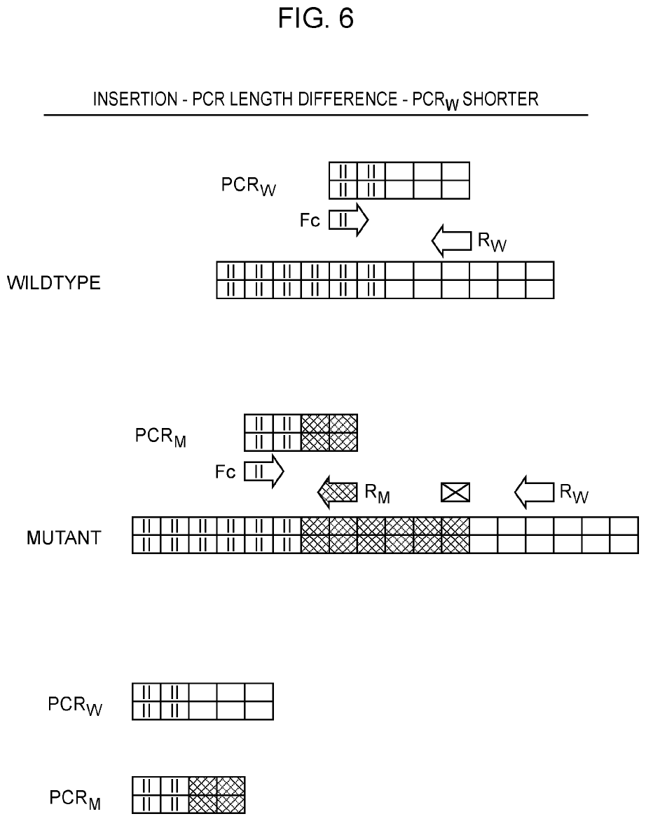

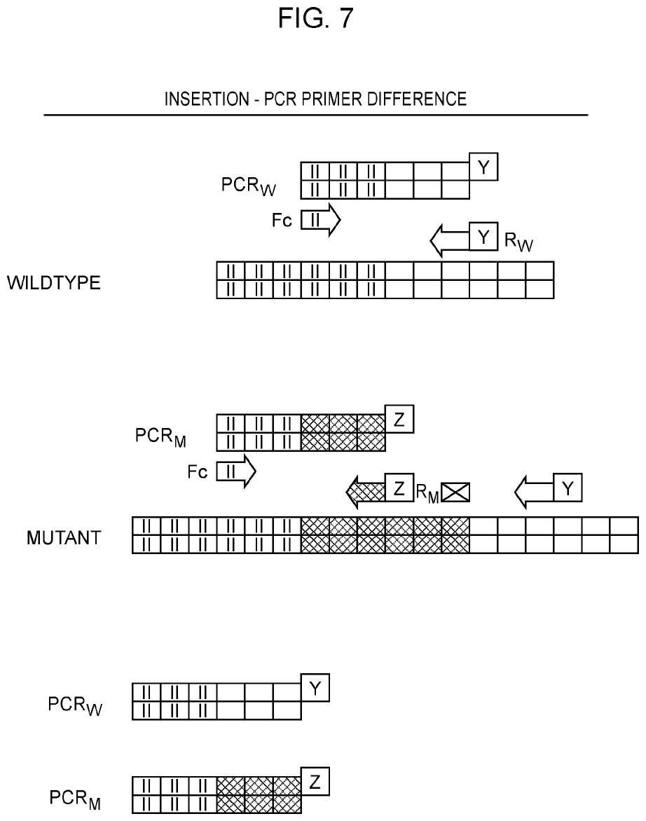

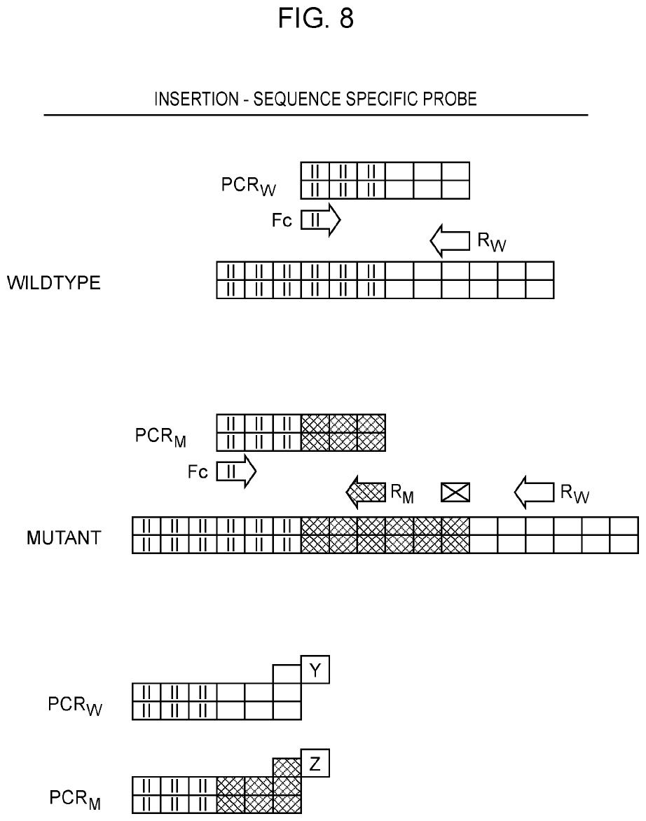

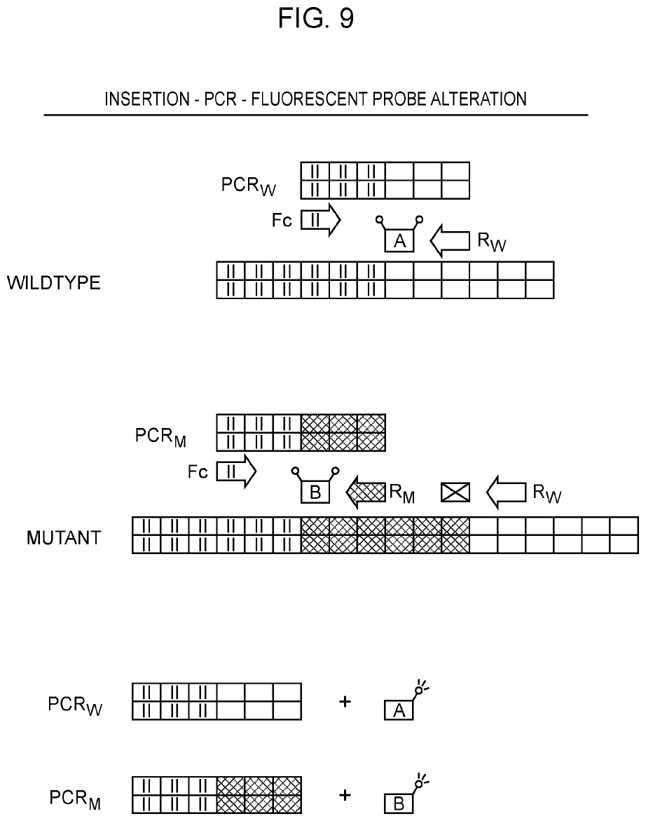

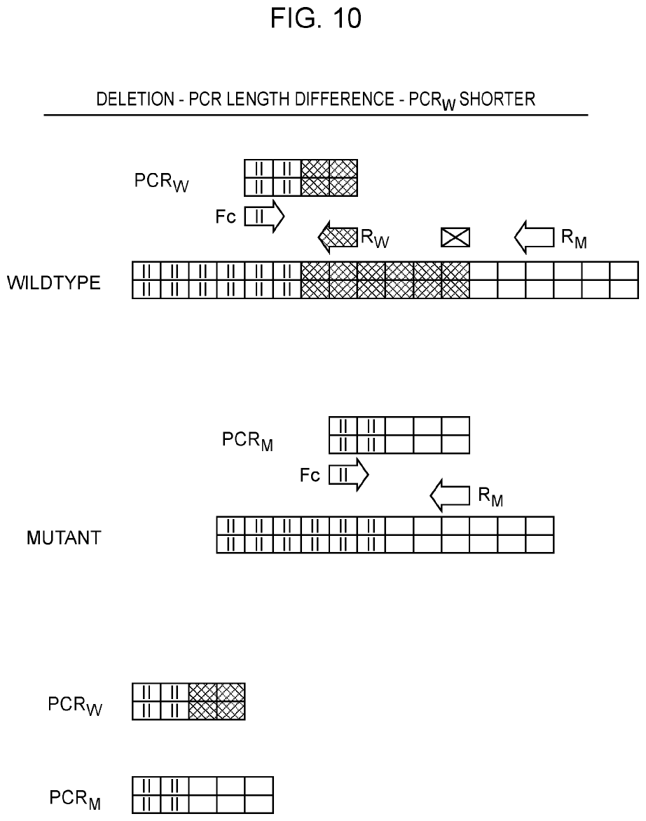

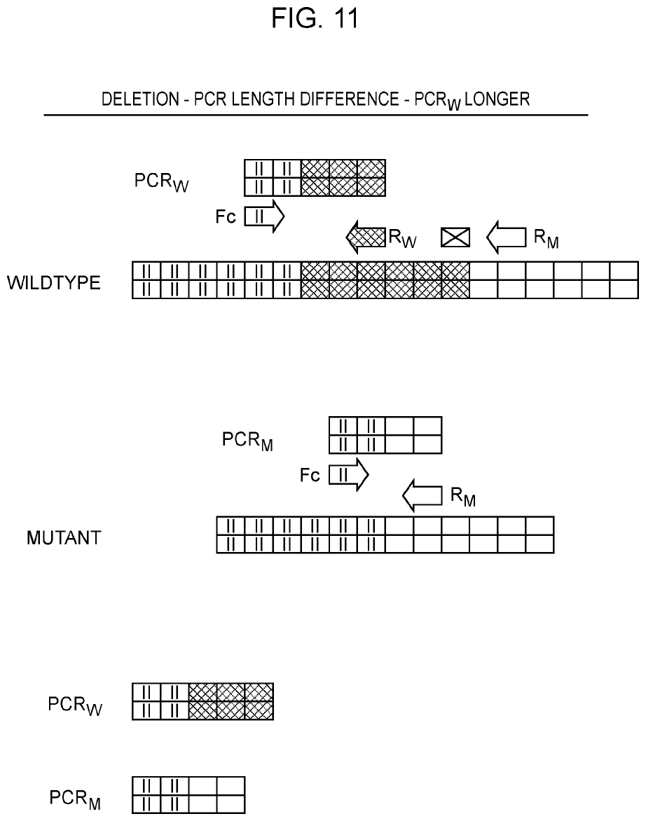

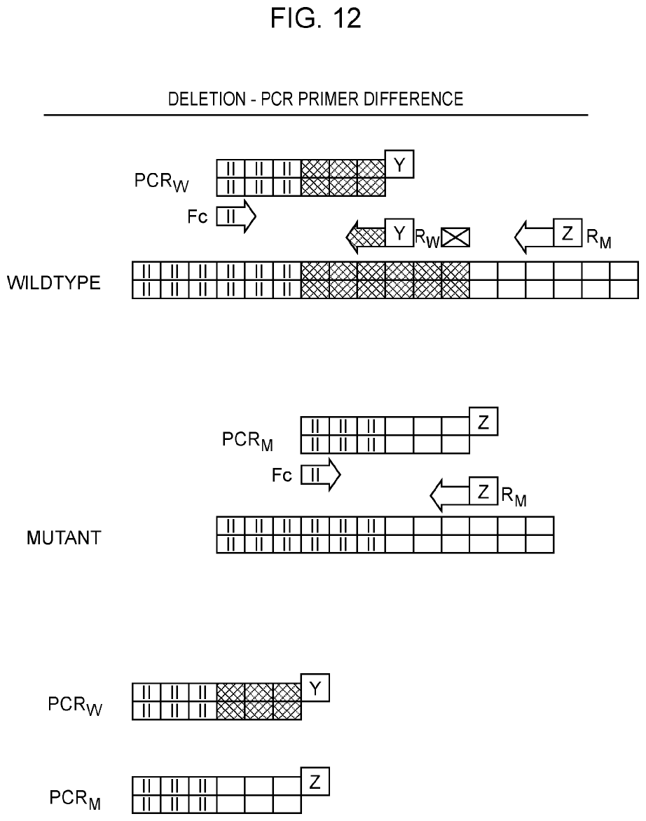

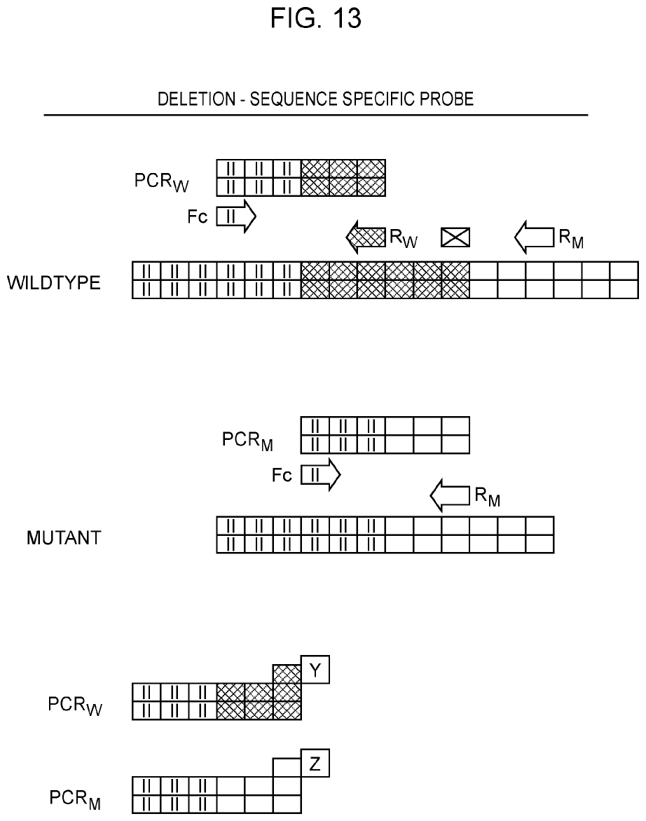

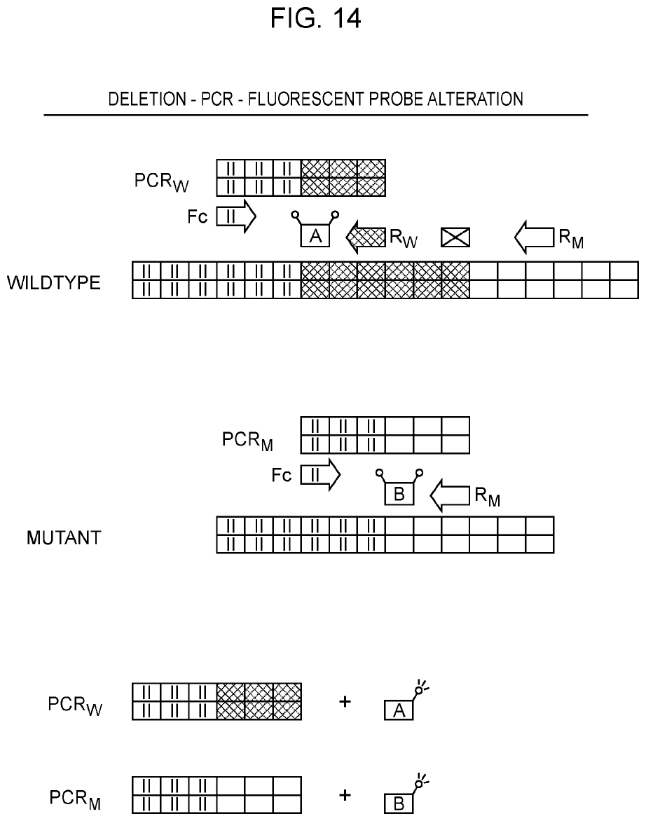

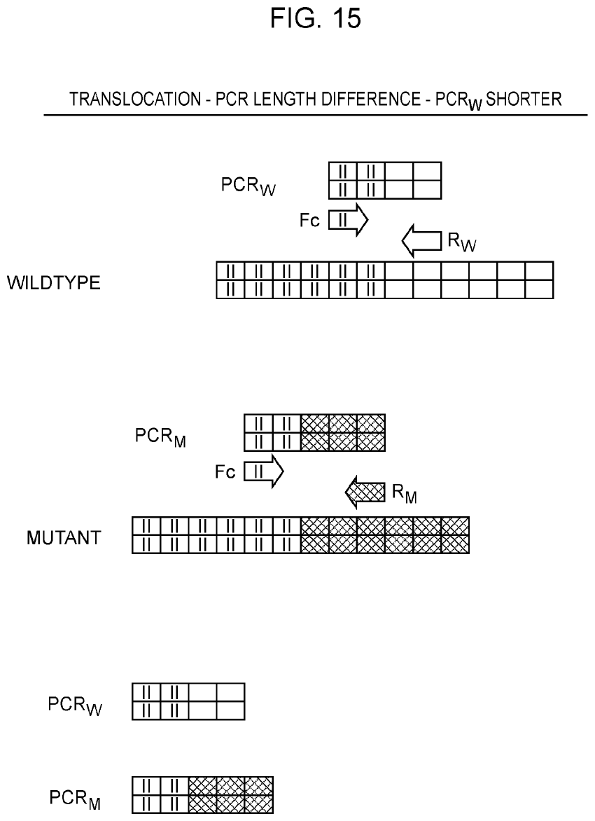

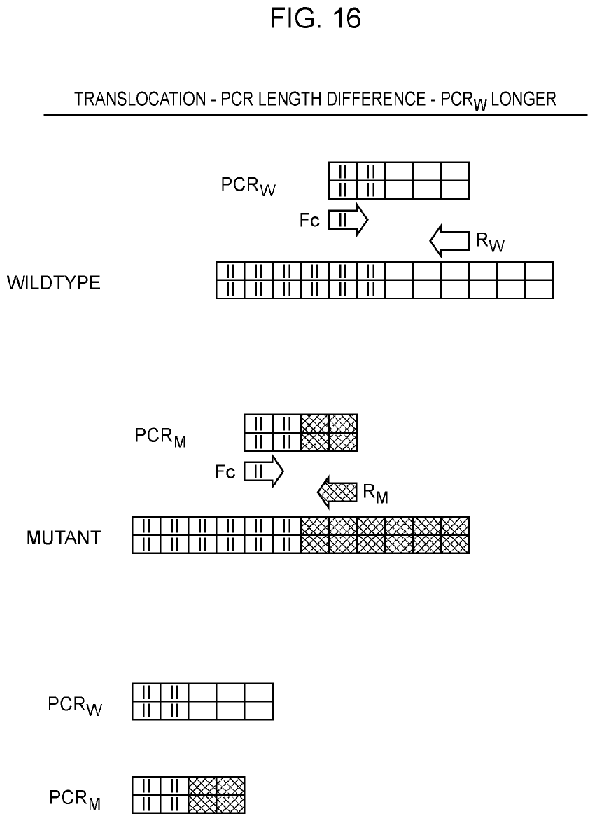

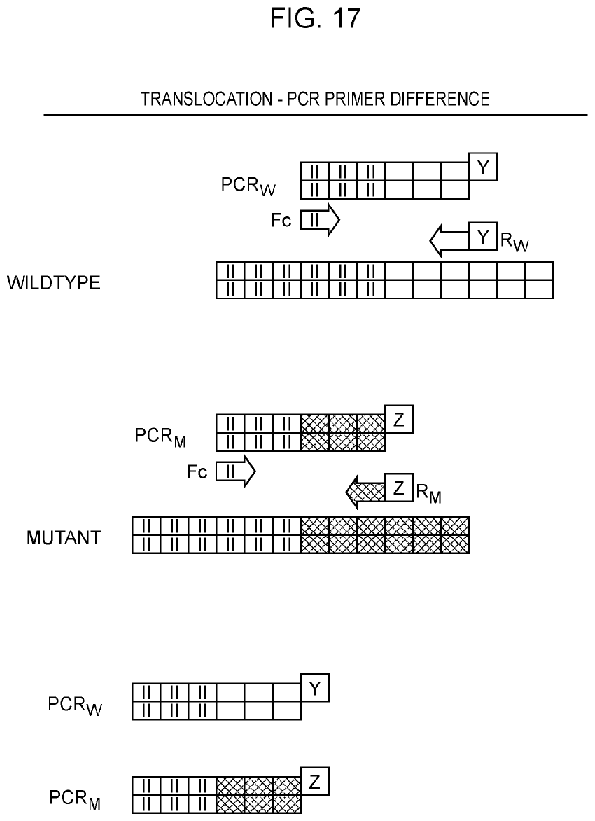

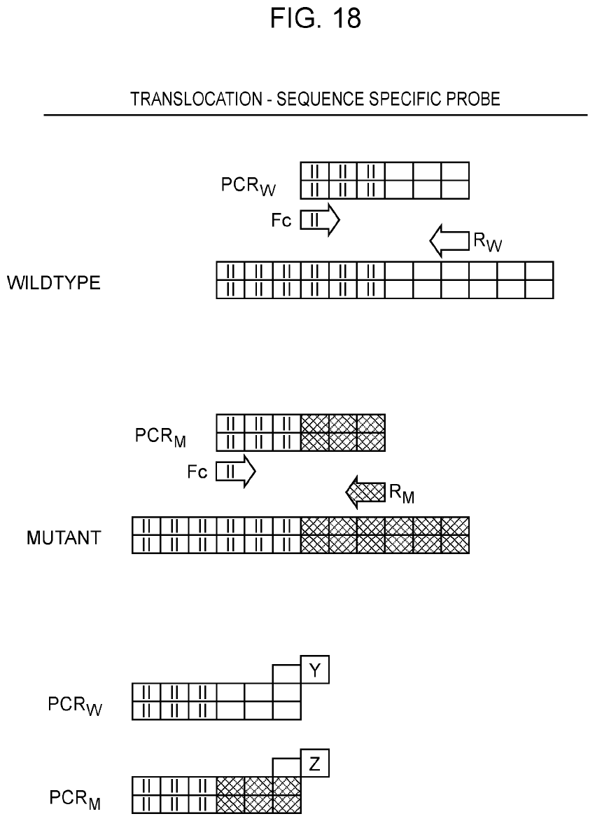

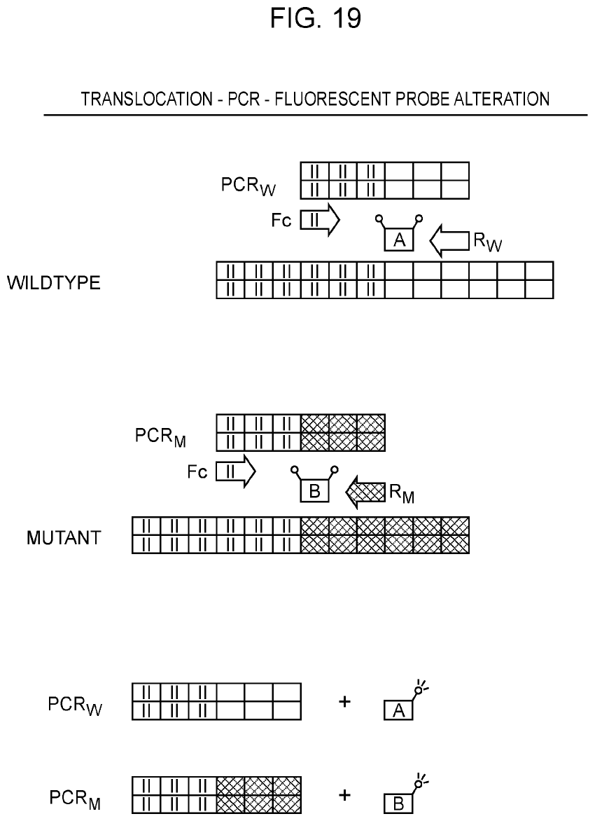

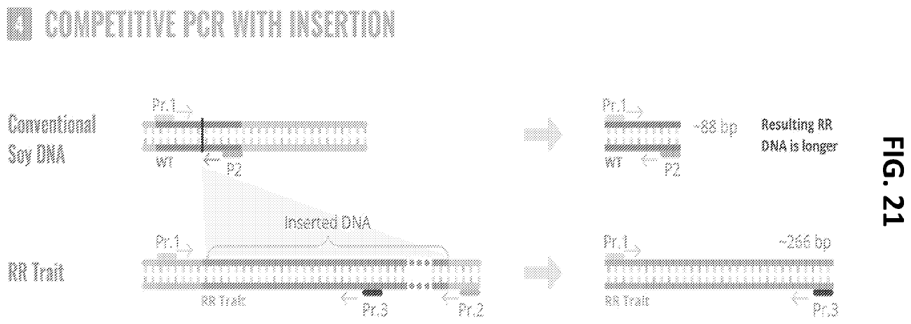

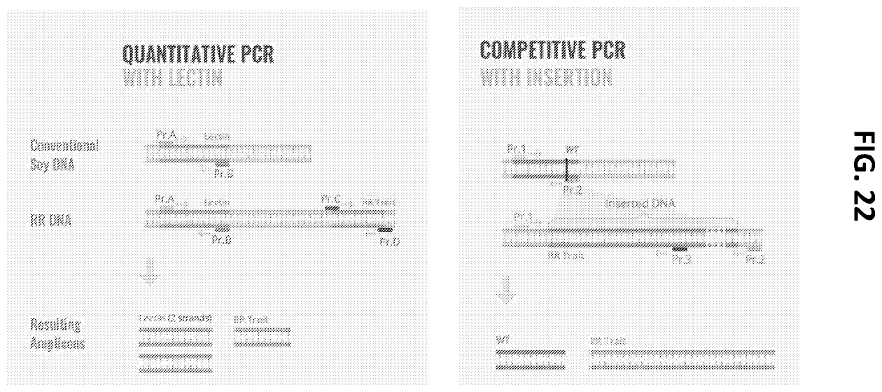

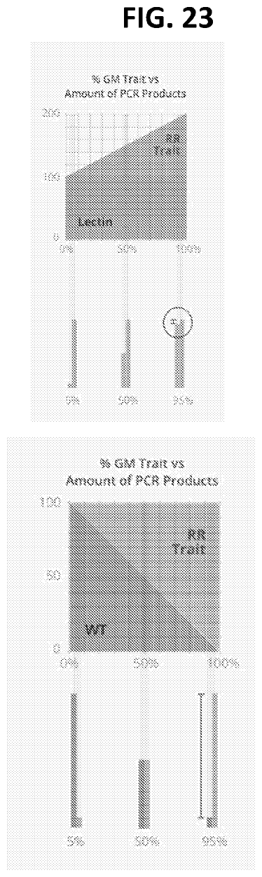

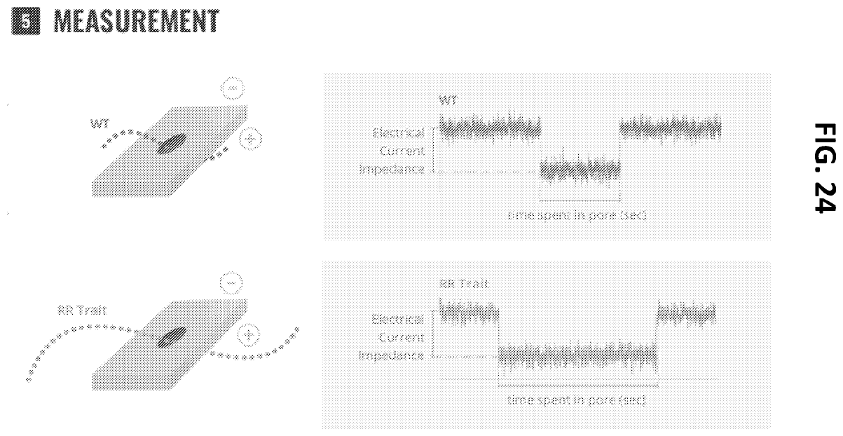

Several embodiments of PCR amplification reactions to distinguish and quantify sequence variants in a sample are shown in in FIGS. 5-19 and FIGS. 21-24. Provided in FIGS. 5-19 are diagrams of embodiments of primer arrangements for quantification of genetic variants, such as insertions, deletions, translocations, duplications, and inversions. As illustrated, identification of distinct genetic variants can be obtained using such features as PCR product length, modification of the primers, sequence specific probes, and probes that are altered during the reaction (such as taqman probes). In preferred embodiments, both amplifications occur in the same reaction volume (unlike digital PCR), and the measurement is only made after the PCR is finished (unlike qPCR).

In some embodiments, the method comprises obtaining a first DNA sequence from a mixed population sample, wherein the first DNA sequence comprises a transgene inserted into the genome (e.g. trait DNA also used interchangeably herein as “variant” DNA). In some embodiments, the method further comprises obtaining a second DNA sequence from the mixed population sample that does not comprise a transgene (e.g. DNA sequence from a non-transgenic organism, also used interchangeably herein as “non-trait”. “non-trait specific” or Wildtype DNA).

In some embodiments, the method comprises designing a set of primers capable of binding specifically to a target sequence to initiate an amplification reaction. In some embodiments, the method comprises designing two oligonucleotide PCR primers that generate PCR amplicons with base pairs ranging from 50-500 base pairs in length. In some embodiments, the method comprises designing two oligonucleotide PCR primers that generate PCR amplicons with base pairs ranging from 50-100 base pairs in length, 100-150 base pairs in length, 150-200 base pairs in length, 200-250 base pairs in length, 250-300 base pairs in length, 300-350 base pairs in length, 350-400 base pairs in length, 400-450 base pairs in length, or 450-500 base pairs in length.

In some embodiments, a first primer (e.g. common oligonucleotide primer) comprises a common DNA. In some embodiments, the common DNA comprises sequences from the trait DNA and the non-trait DNA that are identical, or substantially identical.

In some embodiments, the methods of the present disclosure comprise one or more variants in a mixed sample. In some embodiments, the first or second variant comprises a single nucleotide polymorphism. In some embodiments, the first or second variant comprises a silent mutation, a missense mutation, or a nonsense mutation. In some embodiments, the first or second variant comprises a modified nucleotide or a non-natural nucleotide. In some embodiments, the first or second variant comprises a nucleotide sequence that is genetically modified to introduce one or more traits. In some embodiments, the one or more traits comprise traits resistant to herbicides or pests. In some embodiments, the one or more traits is a soybean that comprises resistance to glyphosate.

In some embodiments, the second primer (e.g. wild type oligonucleotide primer) comprises the non-trait (e.g. wild type non-trait DNA) that crosses the site that is disrupted when the transgene is translocated, inverted, duplicated, deleted, inserted, or any other genetic rearrangement. In some embodiments, the second primer (e.g. oligonucleotide primer) comprises the trait (e.g. variant primer) that crosses the junction in the trait DNA. In some embodiments, the common primer is common to the amplification reaction for both targets, while the third primer is a trait primer (e.g. variant primer) that that crosses the junction in the trait DNA (variant DNA). In some embodiments, the third primer (e.g. wildtype oligonucleotide primer) comprises the non-trait (e.g. wild type non-trait DNA) that crosses the site that is disrupted when the transgene is translocated, inverted, duplicated, deleted, inserted, or any other genetic rearrangement. In some embodiments, the common primer (e.g. first primer) is used to design the non-trait primer (e.g. wildtype oligonucleotide primer).

In some embodiments, the common primer is common to the amplification reaction for both targets, while the first primer is designed to generate, for example, a first amplification product. In some embodiments, the common primer is common to the amplification reaction for both targets, while the third primer is designed to generate, for example, a second amplification product.

In some embodiments, the method comprises a set of primers, wherein the set of primers comprise a first primer that binds specifically to a common sequence on a first strand of said first variant and said second variant from the mixed sample, wherein said first primer is added at a reaction limiting concentration; a second primer that binds specifically to a second strand of said first variant; and a third primer that binds specifically to a second strand of said second variant.

In some embodiments, the sample comprises a region of target genes for which the first primer, second primer, and/or third primer binds. As a non-limiting example, target genes representing the wildtype (SEQ ID NO: 1) or (SEQ ID NO:6) and the mutant variant indicating RR seeds (SEQ ID NO: 2) or (SEQ ID NO:7) are shown below. In bold and underlined are example target sequences for the primers used in the PCR amplification reaction for each:

SEQ ID NO: 1 >Wildtype Glycine max chromosome 2 pos 8001961..8002760
(SEQ ID NO: 1)
ACACTAAATGCATGTTTAGATTAAAGCTTGTAAATTTAAGTTTGAGTTAAATTTA
AGTTAGTAAATTGAGTTTGCAAAAATAACTTAGTGTACTACGAAATTGAGTAGT
TTTCAGACAAAATTTTAGTTGCACATAGTTTTGAGGATAACCAAACTATGTTTAG
CATTCAAAAGTACTTTTTGAACAGTTAACCAAACATGTCCTAAATCATTATAC
ATTAACAAATTCCTTTATTAAAAAAAGTTTAAATATGATTTATAATGTTCATAGT
ATTAAGTTCTGGATTATTAGTTTTTAAAACATTTGATCTATAAGGTTAGTTTTATC
AAGCGGCAAGTCAATCGTGTCGTTCACATCTTTGCAAGAGCACCTCGTTTCTATG
CTAATTACTGCATTTTTTTTATTTTAACCTGTATGTATGATCTTATTTTGAATGAA
ATGCAATAAGTTATTTCTAGTAAAAAAAAATAAACATTTGATAGAAACAAATTA
AAGCATGCAAAAATAACTCATTAGCATCGGTTAAATTGAAGGGTTTGAATAAT
TTGCACAAGGTTCTGAATTCAAATCTTGTTCATTGTAAAAAATAAAGCATGAAA
AAAAGAGGGGCAAAATTTAAACATAAATAATAAGGATTCGGTAAGATCGAGAA
TCGCAATGTAGGGATTCAGATAAAAATATGTTAAGCAGATTGAAGGATAATATA
TATATATATATATATATATATATATATATATATTGTATCTGAAGGATAATATTTT
AAATTTACTGAAGCATAGCTCCAAAATTACGCGGTTTC;
SEQ ID NO: 2 >Mutant MON04032 transgene/chromosome junction B
(SEQ ID NO 2)
TTGAAGATTTAGGAACTTGGGGTTTATGGAAATTGGAATTGGGATTAAGGGTTT
GTATCCCTTGTGCCATGTTGTTAATTTGTGCCATTCTTGAAAGATCTGCTAGAGT
CAGCTTGTCAGCGTGTCCTCTCCAAATGAAATGAACTTCCTTATATAGAGGAAG
GGTCTTGCGAAGGATAGTGGGATTGTGCGTCATCCCTTACGTCAGTGGAGATAT
CACATCAATCCACTTGCTTTGAAGACGTGGTTGGAACGTCTTCTTTTTCCACGAT
GCTCCTCGTGGGTGGGGGTCCATCTTTGGGACCACTGTCGGCAGAGGCATCTTC
AACGATGGCCTTTCCTTTATCGCAATGATGGCATTTGTAGGAGCCACCTTCCTT
TTCCATTTGGGTTCCCTATGTTTATTTTAACCTGTATGTATGATCTTATTTTGAAT
GAAATGCAATAAGTTATTTCTAGTAAAAAAAAATAAACATTTGATAGAAACAAA
TTAAAGCATGCAAAAATAACTCATTAGCATCGGTTAAATTGAAGGGTTTGAAT
AATTTGCACAAGGTTCTGAATTCAAATCTTGTTCATTGTAAAAAATAAAGCATG
AAAAAAAGAGGGGCAAAATTTAAACATAAATAATAAGGATTCGGTAAGATCGA
GAATCGCAATGTAGGGATTCAGATAAAAATATGTTAAGCAGATTGAAGGATAAT
ATATATATATATATATATATATATATATATATATATTGTATCTGAAGGATAATAT
TTTAAATTTACTGAAGCATAGCTCCAAAATTACGCGGTTTC;
SEQ ID NO: 6 >Wildtype Glycine max chromosome 2 pos 7841570..78423692
(SEQ ID NO: 6)
AAGTCCCCATAGATTACATAACCGACAAAAACAATGCCCATATCTAGGAAGCCA
ATACAGTCGATATAAATAACATTAATCCACACCTAAATGTCATAACTCATAAAC
AACCCTAAGCATTAAATTGGAGTCCAAGTACTAGAGAAAGGCTTAATTTCGTAT
TGTAATCTCCCTCAGAATTTCTTAATCTTGTGATCAACAAAGCATATCCTCGTTTT
AAATTCTAAAGGTTATGGCAAAATTCACTGGCATACGAACAATTCATATATCCA
TTCCTATTATATATAGTTGGCAGAAGTACAAGGAGGCGCCAAATAGAAAACACA
AATTGGAACGGTGAAGAGAAAGAAGAGTACCTCGGAGAGAGTTGAGGCGAGA
GATGAGATCGGGAGGGAAGAGATTGGGATCGGAGAAGAACTGTTTGAGGCGAA
TGGCCTGGTCGTCGCGGCCATCGTCGAGAAGTTCGTGAAGAAGCTCGAATGCG
GTGAGAAGGTAGTTCTCTTCCAACAGAAAGTTCACCACGCAATTGCACAGCGA
AGATCTCTCCACGTCCATTTTCTCTCTCTGTCTCTGATCTTAAGCCATTCATTCAA
GACAAGACAAGAGAAGAGAAGAGAAGAGAAGAGAACACTCTCAGTCAGATCGT
GGTTTCAACTTTCAAGACTGTGCTAGCTAGTTAGGTGCCATCTTACATGTTTACT
TTTTTTCTTTATAAGATTAAATTGCTGAATACCATGCTCTCCTGTGTCCAAAGCA
GTACACCCGCGTAAAAATAGATTTCATCGTCCTTTCGATTTTAC;
SEQ ID NO: 7 >Mutant MON04032 transgene/chromosome junction A
(SEQ ID NO: 7)
AATTAAATAAATCAATTACTTCATAAATAATTTTTTTTATAGAATATGTTGACAT
TCTAGCCGGATATAGAACTAATGTAAAGAAACCTTAAAAATTTTGTTTGGAAGA
ATATGTTATTGAAAGACAAATCTAATTAAGTTTATCAGGGTCATTTGTTGAAGAT
AGGAAACCTTCAGCAATTTGAATATTAAGTAACTGCTTCTCCCAGAATGATCG
GAGTTTCTCCTCCTGCTATTACATGAGCAAAAATAAAAAATAAATAAAAGATA
AGATTAAGCTTCAACATGTGAAGGAGTAGTACACTCACCAGTGACCCTAATAGG
CAACAGCATGAAAAAAAATAAAAAAGAATAAAAATAGCATCTACATATAGCTT
CTCGTTGTTAGAAAAACAAAACTATTTGGGATCGGAGAAGAACTGTTTGAGGCG
AATGGCCTGGTCGTCGCGGCCATCGTCGAGAAGTTCGTGAAGAAGCTCGAATGC
GGTGAGAAGGTAGTTCTCTTCCAACAGAAAGTTCACCACGCAATTGCACAGCG
AAGATCTCTCCACGTCCATTTTCTCTCTCTGTCTCTGATCTTAAGCCATTCATTCA
AGACAAGACAAGAGAAGAGAAGAGAAGAGAAGAGAACACTCTCAGTCAGATC
GTGGTTTCAACTTTCAAGACTGTGCTAGCTAGTTAGGTGCCATCTTACATGTTTA
CTTTTTTTCTTTATAAGATTAAATTGCTGAATACCATGCTCTCCTGTGTCCAAAGC
AGTACACCCGCGTAAAAATAGATTTCATCGTCCTTTCGATTTTAC.

In some embodiments, the first primer (e.g. Fc primer or common primer used interchangeably herein) is a primer that is common to the amplification reaction for both targets, while the second primer (e.g. Rw primer or second variant primer) is designed to generate the wildtype PCR product (i.e., amplicon), and the third primer (e.g. Rm primer, trait-specific primer, or second variant primer used intercheably herein) is designed to generate the mutant/variant PCR product. In some embodiments, the common primer (e.g Fc primer or first primer) is a forward primer. In some embodiments, the second primer (e.g second variant primer or Rw primer) is a reverse primer. In some embodiments, the third primer (e.g third variant primer, trait-primer, or Rm primer used interchangeably herein) is a reverse primer. In some embodiments, the mutant PCR product is 222 bp in length, and the wildtype PCR product is 356 bp in length.

In some embodiments, a first primer that binds specifically to a common sequence on a first strand of said first variant and said second variant from the mixed sample, wherein said first primer is added at a reaction limiting concentration. In some embodiments, the common primer is a forward primer. Non-limiting nucleotide sequences of a common primer (e.g. first primer) include, but are not limited to: TCAAACCCTTCAATTTAACCGA (SEQ ID NO:5); AACTACCTTCTCACCGCATTC (SEQ ID NO: 10); CGAGCTTCTTCACGAACTTCTC (SEQ ID NO: 11); ACCGCATTCGAGCTTCTT (SEQ ID NO: 12); CTTTCTGTTGGAAGAGAACTACCT (SEQ ID NO: 13); GAGAGATCTTCGCTGTGCAA (SEQ ID NO: 14); GCAATTGCGTGGTGAACT (SEQ ID NO: 15); AGGCCATTCGCCTCAAA (SEQ ID NO: 16); CACGAACTTCTCGACGATGG (SEQ ID NO: 17); GGCCATTCGCCTCAAACAG (SEQ ID NO: 18); and CCCTTCAATTTAACCGATGCTAAT (SEQ ID NO: 19). In some embodiments, the common primer comprises a nucleotide sequence that is common to the wildtype and the variant (e.g. mutant) nucleotide sequence. In some embodiments, the common primer comprises a nucleotide sequence that is common to a first variant and a second variant nucleotide sequence. In some embodiments, the common primer comprises the nucleotide sequence: TCAAACCCTTCAATTTAACCGA (SEQ ID NO:5). In some embodiments, the common primer comprises the nucleotide sequence: AACTACCTTCTCACCGCATTC (SEQ ID NO: 10). In some embodiments, the common primer comprises the nucleotide sequence: CGAGCTTCTTCACGAACTTCTC (SEQ ID NO: 11). In some embodiments, the common primer comprises the nucleotide sequence: ACCGCATTCGAGCTTCTT (SEQ ID NO: 12). In some embodiments, the common primer comprises the nucleotide sequence: CTTTCTGTTGGAAGAGAACTACCT (SEQ ID NO: 13). In some embodiments, the common primer comprises the nucleotide sequence: GAGAGATCTTCGCTGTGCAA (SEQ ID NO: 14). In some embodiments, the common primer comprises the nucleotide sequence: GCAATTGCGTGGTGAACT (SEQ ID NO: 15). In some embodiments, the common primer comprises the nucleotide sequence: AGGCCATTCGCCTCAAA (SEQ ID NO: 16). In some embodiments, the common primer comprises the nucleotide sequence: CACGAACTTCTCGACGATGG (SEQ ID NO: 17). In some embodiments, the common primer comprises the nucleotide sequence: GGCCATTCGCCTCAAACAG (SEQ ID NO: 18). In some embodiments, the common primer comprises the nucleotide sequence: CCCTTCAATTTAACCGATGCTAAT (SEQ ID NO: 19).

In some embodiments, the set of primers comprises a second primer that binds specifically to a second strand of said first variant. In some embodiments, the second primer is a reverse primer. Non-limiting nucleotide sequences of a second primer include, but are not limited to: CAGTTAACCAAACATGTCCTAAATC (SEQ ID NO: 3); GCCCATATCTAGGAAGCCAATAC (SEQ ID NO: 20); AAGAAGAGTACCTCGGAGAGAG (SEQ ID NO: 8); CCACACCTAAATGTCATAACTCATAAAC (SEQ ID NO: 21); AGATCGGGAGGGAAGAGATT (SEQ ID NO: 22); GTACAAGGAGGCGCCAAATA (SEQ ID NO: 23); TTCGTATTGTAATCTCCCTCAGAAT (SEQ ID NO: 24); TCCAAGTACTAGAGAAAGGCTTAAT (SEQ ID NO: 25); AGGAAGCCAATACAGTCGATATAA (SEQ ID NO: 26); TCACTGGCATACGAACAATTCA (SEQ ID NO: 27); TGGAGTCCAAGTACTAGAGAAAGG (SEQ ID NO: 28); TCCCTCAGAATTTCTTAATCTTGTG (SEQ ID NO: 29); GAACAGTTAACCAAACATGTCCTAA (SEQ ID NO: 30); TTCGTATTGTAATCTCCCTCAGAA (SEQ ID NO: 31); and CAGTTAACCAAACATGTCCTAAATC (SEQ ID NO: 3). In some embodiments, the second primer comprises the nucleotide sequence: CAGTTAACCAAACATGTCCTAAATC (SEQ ID NO: 3). In some embodiments, the second second primer comprises the nucleotide sequence: GCCCATATCTAGGAAGCCAATAC (SEQ ID NO: 20). In some embodiments, the second primer comprises the nucleotide sequence: AAGAAGAGTACCTCGGAGAGAG (SEQ ID NO: 8). In some embodiments, the second primer comprises the nucleotide sequence: CCACACCTAAATGTCATAACTCATAAAC (SEQ ID NO: 21). In some embodiments, the second primer comprises the nucleotide sequence: AGATCGGGAGGGAAGAGATT (SEQ ID NO: 22). In some embodiments, the second primer comprises the nucleotide sequence: GTACAAGGAGGCGCCAAATA (SEQ ID NO: 23). In some embodiments, the second primer comprises the nucleotide sequence: TTCGTATTGTAATCTCCCTCAGAAT (SEQ ID NO: 24). In some embodiments, the primer comprises the nucleotide sequence: TTCGTATTGTAATCTCCCTCAGAA (SEQ ID NO: 31) In some embodiments, the second primer comprises the nucleotide sequence: TCCAAGTACTAGAGAAAGGCTTAAT (SEQ ID NO: 25). In some embodiments, the second primer comprises the nucleotide sequence: AGGAAGCCAATACAGTCGATATAA (SEQ ID NO: 26). In some embodiments, the second primer comprises the nucleotide sequence: TCACTGGCATACGAACAATTCA (SEQ ID NO: 27). In some embodiments, the second primer comprises the nucleotide sequence: TGGAGTCCAAGTACTAGAGAAAGG (SEQ ID NO: 28). In some embodiments, the second primer comprises the nucleotide sequence: TCCCTCAGAATTTCTTAATCTTGTG (SEQ ID NO: 29). In some embodiments, the second primer comprises the nucleotide sequence: GAACAGTTAACCAAACATGTCCTAA (SEQ ID NO: 30).

In some embodiments, the set of primers comprises a third primer that binds specifically to a second strand of said second variant. In some embodiments, the third primer is a reverse primer. Non-limiting nucleotide sequences of a third primer (e.g. third primer) include, but are not limited to: CATCTTCAACGATGGCCTTTC (SEQ ID NO: 4); GGAGTTTCTCCTCCTGCTATTAC (SEQ ID NO: 32); CTCCCAGAATGATCGGAGTTTC (SEQ ID NO: 9); ACACTCACCAGTGACCCTAATA (SEQ ID NO: 33); TGATCGGAGTTTCTCCTCCT (SEQ ID NO: 34); GGTCATTTGTTGAAGATAGGAAACC (SEQ ID NO: 35); AAGGAGTAGTACACTCACCAGT (SEQ ID NO: 36); CCTAATAGGCAACAGCATGAAA (SEQ ID NO: 37); TCAACATGTGAAGGAGTAGTACA (SEQ ID NO: 38); GCATCTACATATAGCTTCTCGTTGT (SEQ ID NO: 39); GTACACTCACCAGTGACCCTAATA (SEQ ID NO: 40); CCCTAATAGGCAACAGCATGAA (SEQ ID NO: 41); and CAACGATGGCCTTTCCTTTATC (SEQ ID NO: 42). In some embodiments, the third primer comprises the nucleotide sequence: CATCTTCAACGATGGCCTTTC (SEQ ID NO: 4). In some embodiments, the third primer comprises the nucleotide sequence: GGAGTTTCTCCTCCTGCTATTAC (SEQ ID NO: 32). In some embodiments, the third primer comprises the nucleotide sequence: CTCCCAGAATGATCGGAGTTTC (SEQ ID NO: 9). In some embodiments, the third primer comprises the nucleotide sequence: ACACTCACCAGTGACCCTAATA (SEQ ID NO: 33). In some embodiments, the third primer comprises the nucleotide sequence: TGATCGGAGTTTCTCCTCCT (SEQ ID NO: 34). In some embodiments, the third primer comprises the nucleotide sequence: GGTCATTTGTTGAAGATAGGAAACC (SEQ ID NO: 35). In some embodiments, the third primer comprises the nucleotide sequence: AAGGAGTAGTACACTCACCAGT (SEQ ID NO: 36). In some embodiments, the third primer comprises the nucleotide sequence: CCTAATAGGCAACAGCATGAAA (SEQ ID NO: 37). In some embodiments, the third primer comprises the nucleotide sequence: TCAACATGTGAAGGAGTAGTACA (SEQ ID NO: 38). In some embodiments, the third primer comprises the nucleotide sequence: GCATCTACATATAGCTTCTCGTTGT (SEQ ID NO: 39). In some embodiments, the third primer comprises the nucleotide sequence: GTACACTCACCAGTGACCCTAATA (SEQ ID NO: 40). In some embodiments, the third primer comprises the nucleotide sequence: CCCTAATAGGCAACAGCATGAA (SEQ ID NO: 41). In some embodiments, the third primer comprises the nucleotide sequence: CAACGATGGCCTTTCCTTTATC (SEQ ID NO: 42).

In some embodiments, modified nucleotides or primers are used in the amplification reaction to facilitate detection and discrimination between amplification products, including as described in International PCT Publication No. WO 2018/183380, “Target Polynucleotide Detection and Sequencing By Incorporation of Modified Nucleotides for Nanopore Analysis,” published Oct. 4, 2018, incorporated by reference in its entirety herein.

In some embodiments, the first primer and the third primer differ in base pair length ranging from 5-50 base pairs, 50-100 base pairs, 100-150 base pairs, 150-200 base pairs, 200-250 base pairs, 250-300 base pairs, 300-350 base pairs, 350-400 base pairs, 400-450 base pairs, or 450-500 base pairs. In some embodiments, the first primer and third primer differ in base pair length by 50 base pairs or more, 100 base pairs or more, 150 base pairs or more, 200 base pairs or more, 250 base pairs or more, 300 base pairs or more, 350 base pairs or more, 400 base pairs or more, 450 base pairs or more, or 500 base pairs or more.

In some embodiments, the method comprises performing an amplification reaction on said mixed sample to generate two amplification products of different length. In some embodiments, the first amplification product is generated from the first and second primer. In some embodiments, the second amplification product is generated from the first and third primer. In some embodiments, the set of primers further comprises a fourth primer and a fifth primer that each each bind to a third strand and a fourth strand, wherein the third primer binds to the third strand. In some embodiments, the performing the amplification reaction on said mixed sample further generates a third amplification product and a fourth amplification product.

In some embodiments, the first amplification product (e.g. amplicon product or PCR product used interchangeably herein), second amplification product, third amplification product, and/or fourth amplification product (e.g. amplicon product) differ in length ranging from 5-50 base pairs, 50-100 base pairs, 100-150 base pairs, 150-200 base pairs, 200-250 base pairs, 250-300 base pairs, 300-350 base pairs, 350-400 base pairs, 400-450 base pairs, or 450-500 base pairs. In some embodiments, the first amplification product, second amplification product, third amplification product, and/or fourth amplification product differ in base pair length by 50 base pairs or more, 100 base pairs or more, 150 base pairs or more, 200 base pairs or more, 250 base pairs or more, 300 base pairs or more, 350 base pairs or more, 400 base pairs or more, 450 base pairs or more, or 500 base pairs or more. In some cases, the first amplification product and the second amplification product that differ in length provide for relative quantification of the first and second amplification products following end-point PCR. In some embodiments, the first amplification product, second amplification product, third amplification product, and/or fourth amplification product each comprise base pair lengths ranging from 100 base pairs-150 base pairs in length, 150-200 base pairs in length, 200-250 base pairs in length, 250-300 base pairs in length, 300-350 base pairs in length, 350-400 base pairs in length, 400-450 base pairs in length, or 450-500 base pairs in length. In some embodiments, the first amplification product, second amplification product, third amplification product, and/or fourth amplification product each comprise a base pair length of 100 base pairs or more, 150 base pairs or more, 200 base pairs or more 250 base pairs or more 300 base pairs or more 350 base pairs or more, 400 base pairs or more 450 base pairs or more or 500 base pairs or more. In some embodiments, the first amplification product, second amplification product, third amplification product, and/or fourth amplification product is about 222 base pairs (bp) in length. In some embodiments, the first and/or second amplification product is about 356 bp in length. In some embodiments, the first amplification product, second amplification product, third amplification product, and/or fourth amplification product is about 298 bp in length. In some embodiments, the first amplification product, second amplification product, third amplification product, and/or fourth amplification product is about 153 bp in length. In some embodiments, the first and/or second amplification product is about 219, 298, 180, 277, 369, 258, 217, 159, 110, 140, 124, 211, 222, 398, 153, 104, 391, 400, 392, 282, 226, 311, 267, 354, or 356 bp in length. In some embodiments, the first amplification product has a length that is greater than the second amplification product. In some embodiments, the first amplification product has a length that is less than the second amplification product. In some embodiments, the first amplification product and the second amplification product have the same length. In some embodiments, the third amplification product has a length that is greater than the fourth amplification product. In some embodiments, the third amplification product has a length that is less than the fourth amplification product. In some embodiments, the third amplification product and the fourth amplification product have the same length. In some embodiments, the four amplification products are each of different lengths. In some embodiments, the four amplification products are each the same lengths. In some embodiments, the four amplification products are of three different lengths, with two amplification products being the same length. In some embodiments, the third amplification product is generated from the fourth primer and the third primer and said fourth amplification product is generated from the fourth primer and the fifth primer.

In some embodiments, the method comprises extracting DNA from one or more organisms of the same population (e.g. one or more seeds of a soybean) to create a mixed sample. In some embodiments, a mixed sample comprises a population of mixed DNA extracts from wildtype and/or variant genomes of organisms, such as, but not limited to cells, viruses, agricultural plants or seeds, and the like. In some embodiments, the sample comprises 0% variants (e.g. traits), 5% variants, 10% variants, 15% variants, 20% variants, 25% variants, 30% variants, 35% variants, 40% variants, 45% variants, 50% variants, 55% variants, 60% variants, 65% variants, 70% variants, 75% variants, 80% variants, 85% variants, 90% variants, 95% variants, or 100% variants of the mixed sample. In some embodiments, the sample comprises 0% non-variants (e.g. non-traits), 5% non-variants, 10% non-variants, 15% non-variants, 20% non-variants, 25% non-variants, 30% non-variants, 35% non-variants, 40% non-variants, 45% non-variants, 50% non-variants, 55% non-variants, 60% non-variants, 65% non-variants, 70% non-variants, 75% non-variants, 80% non-variants, 85% non-variants, 90% non-variants, 95% non-variants, or 100% non-variants of the mixed sample. In some embodiments, the mixed sample comprises a percentage of variants ranging from 0-10%, 10-20%, 20-30%, 30-40%, 40-50%, 50-60%, 60-70%, 70-80%, 80-90%, or 90-100%. In some embodiments, the mixed sample comprises a percentage of non-variants ranging from 0-10%, 10-20%, 20-30%, 30-40%, 40-50%, 50-60%, 60-70%, 70-80%, 80-90%, or 90-100%. In some embodiments, the mixed sample comprises 50% of variants, and 50% non-variants. In some embodiments, the mixed sample comprises 100% of variants, and 0% non-variants. In some embodiments, the mixed sample comprises 0% of variants, and 100% non-variants. In some embodiments, the mixed sample contains DNA extracts from 5 or more samples, 10 or more samples, 20 or more samples, 30 or more samples, 40 or more samples, 50 or more samples, 60 or more samples, 70 or more samples, 80 or more samples, 90 or more samples, 100 or more samples, 200 or more samples, 300 or more samples, 400 or more samples, 500 or more samples, 600 or more samples, 700 or more samples, 800 or more samples 900 or more samples, 1000 or more samples, 1500 or more samples, 2000 or more samples, 2500 or more samples, 3000 or more samples, 3500 or more samples, 4000 or more samples, 4500 or more samples, 5000 or more samples, 5500 or more samples, 6000 or more samples, 6500 or more samples, 7000 or more samples, 7500 or more samples, 8000 or more samples 8500 or more samples, 9000 or more samples 9500 or more samples, or 10,000 or more samples. In some embodiments, the samples are DNA extracts derived from a population of plants, agricultural seeds, such as, but not limited to wildtype and/or genetically modified soybean seeds, fruit seeds, vegetable seeds, or any other argricultural seeds. In some embodiments, the samples are DNA extracts derived from a population of wildtype and/or genetically modified eukaryotic cells, prokaryotic cells, mammalian cells, non-mammalian cells, yeast cells, insect cells, human cells, plant cells, mold, fungus, virus, protozoan, an animal a human, and the like. In some embodiments, the mixed sample comprises DNA extracts from target genes from an organism of interest.

In some embodiments, the sample is derived from an organism or a population of organisms. In some embodiments, the relative amount of genetic variants is used to determine a zygosity of said organism. In some embodiments, the organism is suspected of being a genetically modified organism. In some embodiments, at least one of said genetic variants is recombinantly engineered.

In some embodiments, the quantification determines a zyogosity of an organism comprising said genetic variants. In some embodiments, at least one of said genetic variants comprises a recombinantly engineered gene. In some embodiments, at least one of said genetic variants comprise an inserted sequence. In some embodiments, at least one of said genetic variants comprises a genetic rearrangement. In some embodiments, the sample is derived from a virus, a protozoan, a fungus, a mold, a plant, an animal, or a human.

In some embodiments, the methods of the present disclosure include mixing a sample with a set of primers capable of binding specifically to a target sequence to initiate an amplification reaction. For example, a sample containing a DNA can be mixed with a set of primers.

In some embodiments, the method comprises performing an amplification reaction on said mixed sample, for example, to generate one or more, two or more, three or more, four or more, five or more, six or more, seven or more, eight or more, nine or more, or ten or more amplification products of different length. In some embodiments, the method comprises performing an amplification reaction on said mixed sample to generate two amplification products of different length, wherein said first amplification product is generated from the first primer and second primer, and wherein the second amplification product is generated from the first and third primer. In some embodiments, said performing an amplification product comprises mixing the sample with amplification reaction components and incubating under conditions that promote DNA amplification. In some embodiments, the amplication reaction reagents can include, but are not limited to:free primers, dNTPs (deoxynucleotide triphosphates, dATP, dGTP, dCTP, dTTP), polymerase enzymes (e.g., Taq or Pfu), salts (Magnesium chloride, Magnesium Sulfate, Ammonium sulfate, sodium chloride, potassium chloride), BSA (bovine serum albumin) stabilizer, and detergents (e.g., triton X-100). Amplification reactions can include, but are not limited to, e.g., PCR, ligase chain reaction (LCR), transcription mediated amplification (TMA), reverse transcriptase initiated PCR, DNA or RNA hybridization techniques, sequencing, isothermal amplification, and loop-mediated isothermal amplification (LAMP). Techniques of amplification to generate an amplicon from a target polynucleotide sequence are well known to one of skill in the art.

In some embodiments, mixing comprises mixing the first primer at a reaction limiting concentration. In some embodiments, the reaction limiting concentration comprises a concentration of the first primer ranging from 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.15, 1.2, 1.25, 1.3, 1.35, 1.4, 1.45, 1.5, 1.55, 1.6, 1.65, 1.7, 1.57, 1.8, 1.85, 1.9, 1.95, or 2.0 μL at 10, 15, 20, 25, 30, 35, 40, 45, 50, 55, 60, 65, 70, 75, 80, 85, 90, 95, 100, 105, 110, 115, 120, 125, 130, 135, 140, 145, or 150 μM. In some embodiments, the second primer comprises a concentration ranging from 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.15, 1.2, 1.25, 1.3, 1.35, 1.4, 1.45, 1.5, 1.55, 1.6, 1.65, 1.7, 1.57, 1.8, 1.85, 1.9, 1.95, or 2.0 μL at 10, 15, 20, 25, 30, 35, 40, 45, 50, 55, 60, 65, 70, 75, 80, 85, 90, 95, 100, 105, 110, 115, 120, 125, 130, 135, 140, 145, or 150 μM. In some embodiments, the third primer comprises a concentration ranging from 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.15, 1.2, 1.25, 1.3, 1.35, 1.4, 1.45, 1.5, 1.55, 1.6, 1.65, 1.7, 1.57, 1.8, 1.85, 1.9, 1.95, or 2.0 μL at 10, 15, 20, 25, 30, 35, 40, 45, 50, 55, 60, 65, 70, 75, 80, 85, 90, 95, 100, 105, 110, 115, 120, 125, 130, 135, 140, 145, or 150 μM. In some embodiments, the first, the second, and the third primers comprise the same concentration relative to each other. In some embodiments, the first, second, and third primers comprise different concentrations relative to each other. In some embodiments, the concentration of the first, second, and third primer is 0.5 μL at 30 μM. In some embodiments, the concentration of the first primer is 1 μL. In some embodiments, the concentration of the second primer is 1 μL at 100 μM. In some embodiments, the concentration of the third primer is 1 μL at 100 μM. In some embodiments, the concentration of the first primer and the third primer is 1 μL at 100 μM, and the concentration of the second primer is 0.75 μL at 100 μM.

In some embodiments, the methods of the present disclosure comprise detecting at least two distinct signals corresponding to the first amplification product and the second amplification product. In some embodiments, the method comprises detecting at least two distinct signals corresponding to the first amplification product and the second amplification product.

In some embodiments, the first amplification product, the second amplification product, the third amplification product, and/or the fourth amplification product are detected using capillary electrophoresis, gel electrophoresis, sequence specific fluorescent probes, or separation of the amplification products with affinity tags on the set of primers.

In some embodiments, the first amplification product and second amplification product are detected using a nanopore device. In some embodiments, the method comprises loading a first amplification product and/or a second amplification product on the nanopore device. In some embodiments, the method comprises loading a third amplification product and/or a fourth amplification product on the nanopore device. In some embodiments, the method comprises loading a first amplification product and/or a second amplification product on into a chamber of the device (e.g. a middle chamber). In some embodiments, the method comprises loading a third amplification product and/or a fourth amplification product on into a chamber of the device (e.g. a middle chamber). In some embodiments, the method comprises loading a first amplification product and/or a second amplification product on into a channel of the nanopore device. In some embodiments, the method comprises loading a third amplification product and/or a fourth amplification product on into a channel of the device. In some embodiments, the detecting comprise detecting a first signal corresponding to the first amplification product as the first amplification product translocates through at least one nanopore. In some embodiments, the detecting comprise detecting a second signal corresponding to the second amplification product as the second amplification product translocates through at least one nanopore. In some embodiments, the detecting comprise detecting a third signal corresponding to the third amplification product as the third amplification product translocates through at least one nanopore. In some embodiments, the detecting comprise detecting a fourth signal corresponding to the fourth amplification product as the fourth amplification product translocates through at least one nanopore.

In some embodiments, the method comprises quantifying the relative amount of the first and the second amplification products based on said detected signals. In some embodiments, quantifying comprises applying a principal component analysis to the detected signals.

In some embodiments, the method comprises determinating the percentage by weight of non-trait specific and/or trait-specific organisms (e.g. seeds, cells, etc.) in a mixed population. In some embodiments, the method comprises distinguishing the two amplification products by their difference in PCR length using the characteristics of the electrical signal as the amplification products pass through a solid-state nanopore. In some embodiments, the method comprises calculating the percentage by weight in the starting mixed sample using the electrical signals detected. In some embodiments, a control (e.g. reference samples) are performed using the methods as described herein in order to calibration equations in order to calculate the percentage by weight in the starting mixed sample using electrical signals detected.

In some embodiments, the methods of the present disclosure of quantifying the relative amount of genetic variants in the mixed sample can be completed during a time period of about 30 minutes or less, about 25 minutes or less, about 20 minutes or less, about 15 minutes or less, about 10 minutes or less, or about 5 minutes or less. In some embodiments, the methods of the present disclosure of quantifying the relative amount of genetic variants in the mixed sample can be completed during a time period ranging from 1-2 minutes, 2-5 minutes, 5-10 minutes, 10-15 minutes, 15-20 minutes, or 25-30 minutes.

After the PCR has reached its end point, the method comprises measuring a detected signal which can distinguish one amplicon type from the other, based upon properties specified by their associated specific primer. The measured property specified by a primer can be the length of the amplicon, the unique DNA sequence between the specific primer binding site and the common primer, or a physical or chemical modification to the primer. In some embodiments, the method comprises making a ratio of the two amplicons based on the two values obtained from the measurements of the two amplicons.

In some embodiments, the method comprises using the ratios obtained from a number of tests on combined DNA template samples from training populations with known frequencies of the genetic rearrangement, for generating a lookup table (or an equation fitting the data). In some embodiments, the method comprises using the table or equation for calculating calculate the frequency of the genetic rearrangement in the combined DNA template samples from unknown populations, using the ratios obtained from a test. In some embodiments, the method comprises creating a reference data set for making a calibration equation. In some embodiments, the reference data is generated from any amount of test PCRs. In some embodiments, the amount of test PCRs is at least a single reaction with the 50% trait-extract mix.

In some embodiments, the frequency of the genetic rearrangement in the combined DNA template sample is correlated to the fraction, or amount, of any physical or chemical properties of the population. Accordingly, a lookup table can also be generated to calculate that fraction, or amount, from the ratios obtained from a test. For example, if the frequency of the genetic rearrangement in the combined DNA template sample is correlated to the fraction of the total mass of organisms that contain the genetic rearrangement, then a lookup table (or equation) can be generated to calculate the fraction of the total mass of the population containing the genetic rearrangement directly from the ratio of the measurements of the two amplicons at the end-point of the PCR.

In some embodiments, training data is used to generate the lookup table. The training data can be measured while using a defined reproducible procedure for all processing steps of the training populations, and the same procedure is used to process test populations. All DNA purification and PCR amplification procedures are compatible with the method, as long as the entire procedure reproducibly generates two distinguishable amplicons at a ratio correlated to the frequency of the genetic rearrangement in the combined DNA template samples of the training populations.

Because only reproducibility is required, the accuracy is not affected by inhibitors or impurities, as long as they were also present at the same (or similar) levels in the training set. Similarly, the accuracy is not affected by the exact concentration of the starting DNA template, or any other components of the PCR mixture, as long as they were at the same (or similar) levels while generating the training set and when performing the test.

All of the conventional methods used to determine the frequency of a genetic rearrangement within a population rely upon independently measuring the level of a genetic rearrangement, and the level of an unrelated reference genetic locus that is set as the 100% level. The frequency of the rearrangement in the population is then directly calculated as a ratio of the level of the rearrangement to the level of the independently determined reference locus. A reference locus does not need to be absolutely conserved at the DNA level, and it can be the sum of all of the variants at a particular genetic locus in the population (and that reference locus could be the site of the rearrangement being tested).

The method, as described herein according to some embodiments, relies instead on the interaction of two PCR amplifications, which can occur in the same location, and share one common primer. The other methods require independent PCR reactions, with one serving as the 100% reference. The independent reactions may occur within the same tube, provided that excess reagents are present. In the method described herein, according to some embodiments, neither of the two DNA amplicons is an independent reference, and the sum of the DNA amplicons is not set as the 100% reference level. Although duplex amplification reactions are described herein, in some embodiments, a single reaction mixture can include three or more distinct genetic variants as well.

The measurement of the relative amplicon levels can be made using any property specified by the specific primers. The measurement could be made while the two amplicon types are still in the same location (for example, using fluorescence in a tube, well, or droplet), after they are separated by their unique properties (for example, using UV absorbance of bands on a gel), while individual molecules are being transferred through a sensor (for example, using the electrical signal while traversing a nanopore), or after each molecule has been isolated (for example, using single molecule DNA sequencing).

In some embodiments, methods of determining relative estimates of the quantity of the genetic variants in a sample is performed as described in International PCT Publication No. WO 2018/081178, “Fractional Abundance of Polynucleotide Sequences in a Sample,” published May 3, 2018, incorporated by reference in its entirety herein.

In some embodiments, the amplification reaction is limited to align amplification rates of said first and second variants. In some embodiments, at least one component of the amplification reaction is provided at a limiting reaction to align amplification rates of said first and second variants. In some embodiments, the amplification reaction is inhibited by PCR conditions, a PCR blocking oligonucleotide, or sequence specific cleavage of the DNA template.

In some embodiments, the method further comprises amplifying a control gene in said sample, and quantifying one or both of said amplification products relative to said amplified control gene.

In some embodiments, the amplification reaction is selected from polymerase chain reaction (PCR) or isothermal amplification. Various PCR-based methods are conventionally known. In some embodiments, the amplification reaction is performed with a touch thermocycler. In some embodiments, the performing the amplification reaction comprises amplifying the amplification reaction mixture with a thermocycler at a temperature of ranging from 95° C. for about 30 seconds, followed by about 35 cycles at a temperature of about 95° C. for about 5 seconds, followed by a temperature of about 72° C. for about 10 seconds, followed by 72° C. for about 30 seconds. In some embodiments, the amplification reaction is performed with a touch thermocycler. In some embodiments, the performing the amplification reaction comprises amplifying the amplification reaction mixture with a thermocycler at a temperature of ranging from 98° C. for about 5 seconds, followed by about 35 cycles at a temperature of about 98° C. for about 1 second, followed by a temperature of about 55° C. for about 1 second, followed by 75° C. for about 3 seconds. In some embodiments, method further comprises merging the triplicate amplification reactions to a volume ranging from about 20-25 μL, 25-30 μL, 30-35 μL, 35-40 μL, 40-45 μL, 45-50 μL, 50-55 μL, 60-65 μL, 65-70 μL, 70-75 μL, 75-80 μL, 80-85 μL, 85-90 μL, 90-95 μL, or 95-100 μL before analysis. In some embodiments, performing the amplification reaction occurs for a time period ranging from 1-2 minutes, 2-3 minutes, 3-4 minutes, 4-5 minutes, 5-6 minutes, 6-7 minutes, 7-8 minutes, 8-9 minutes, or 9-10 minutes. In some embodiments, performing the amplification reaction occurs for a time period of about 10 minutes or less, 9 minutes or less 8 minutes or less, 7 minutes or less, 6 minute or less, 5 minutes or less, 4 minutes or less, 3 minutes or less, 2 minutes or less, or 1 minute or less.

In some embodiment, the method of the present disclosure comprises diluting the amplification products (e.g. first amplification product, second amplification product, third amplification product, and/or fourth amplification product) in a recording buffer (e.g. sensing solution). Non-limiting examples of buffer solutions that can be added to a sensing solution are a TRIS-HCl, a Borate, a CHES, a Bis-tris propane, a CAPS, a potassium phosphate, a TRIS, or a HEPES.

### Sensing Solutions

Aspects of the present method include mixing and/or diluting an amplicon product post-amplification with a sensing solution (e.g. nanopore recording buffer). In some embodiments, the amplification products in the recording buffer is then loaded onto a nanopore device. In some embodiments, the sensing solution is a buffer. In some embodiments, the sensing solution comprises a polyether agent. In some embodiments, the polyether agent is a (poly)ethylene glycol. In some embodiments, the polyether agent is (Poly)propylene Glycol. In some embodiments, the polyether agent is (Poly)butylene Glycol. In some embodiments, the polyether agent is (Poly)alkylene Glycol Ether.

(Poly)ethylene Glycol Agents

The disclosure provides various (poly)ethylene glycol (PEG)-based sensing solutions that can be used for detection or characterization of a biomolecule in a sample using a nanopore device.

In some embodiments, the polyether agent is of Formula (I):

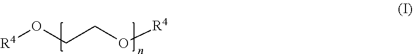

where n is 1-30; and each R⁴ is independently H, alkyl or a terminal group.

In some embodiments of Formula (I), each R⁴ is H. In some embodiments of Formula (I), each R⁴ is alkyl, such as C₍₁₋₆₎ alkyl. In some cases, each R⁴ is methyl. In some embodiments of Formula (I), n is 2-30, such as 3-30, 4-30, 5-30, 6-30, 7-30, 8-30, 10-30, or 10-20. In some embodiments of Formula (I), n is 1. In some embodiments of Formula (I), n is 2-25, such as 2-20, 2-18, 2-16, 2-15, 2-14, 2-13, 2-12, 2-10, 2-8 or 2-6.

In some embodiments of Formula (I), n is 1 and each R⁴ is H (e.g., ethylene glycol). In some embodiments of Formula (I), n is 2 and each R⁴ is H (e.g., diethylene glycol). In some embodiments of Formula (I), n is 3 and each R⁴ is H (e.g., triethylene glycol). In some embodiments of Formula (I), n is 1 and each R⁴ is methyl. In some embodiments of Formula (I), n is 2 and each R⁴ is methyl. In some embodiments of Formula (I), n is 3 and each R⁴ is methyl. In some embodiments of Formula (I), n is 4 and each R⁴ is H (e.g., tetraethylene glycol). In some embodiments, of Formula (I), n is 4 and each R⁴ is methyl (e.g., tetraethylene glycol dimethyl ether).

In some embodiments of Formula (I), the polyether agent is a (poly)ethylene glycol or (poly)ethylene glycol ether having a molecular weight in the range of about 120 to 3000. In some embodiments of Formula (I), the polyether has a molecular weight of 3000 or less, such as, 2500 or less, 2000 or less, 1500 or less, or 1000 or less. It is understood that any of the molecular weights described herein can refer to an average molecular weight due to polydispersity of polyether agents, i.e., such polymers can include molecules with a distribution of molecular weights that can depends on their method of preparation. In some embodiments of Formula (I), the polyether agent has a molecular weight in the range of 100-120, 120-140, 140-160, 160-180, 180-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2200, 2200-2400, 2400-2600, 2600-2800, 2800-3000, 3000-4000, 4000-5000, 5000-6000, 6000-7000, or 7000-8000. It is understood that the size or molecular weight of the particular polyether agent selected for use in the sensing solution can be tailored to provide a desirable sensitivity or accuracy of detection and depends on a variety of conditions, such as the target analyte (e.g., biomolecule), the analyte probe (if utilized) and probe's physical characteristics or chemistry.

In some embodiments of Formula (I) the polyether agent is PEG 120-160 molecular weight. In some embodiments of Formula (I), the polyether agent is PEG 160-200 molecular weight. In some embodiments of Formula (I), the polyether agent is PEG 200-400 molecular weight. In some embodiments of Formula (I), the polyether agent is PEG 200-600 molecular weight. In some embodiments of Formula (I), the polyether is PEG 3000 molecular weight or less.

(Poly)propylene Glycol Agents

The disclosure provides various sensing solutions comprising (poly)propylene glycol (PPG) for the detection and characterization of a biomolecule in a sample using a nanopore device.

In some embodiments, the polyether agent is of Formula (II):

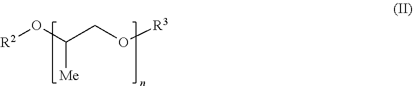

where n is 1-30; and R² and R³ are each independently H, alkyl or a terminal group.

In some embodiments of Formula (II), R² and R³ are each H. In some embodiments of Formula (II), each R² and R³ are each alkyl, such as C₍₁₋₆₎ alkyl. In some cases, R² and R³ are each methyl. In some embodiments of Formula (II), n is 2-30, such as 3-30, 4-30, 5-30, 6-30, 7-30, 8-30, 10-30, or 10-20. In some embodiments of Formula (II), n is 1. In some embodiments of Formula (II), n is 2-25, such as 2-20, 2-18, 2-16, 2-15, 2-14, 2-13, 2-12, 2-10, 2-8, or 2-6.

In some embodiments of Formula (II), n is 2, and R² and R³ are each H (e.g., dipropylene glycol). In some embodiments of Formula (II), n is 3, and R² and R³ are each H (e.g., tripropylene glycol). In some embodiments of Formula (II), n is 3, one of R² and R³ is H, and the other of R² and R³ is methyl. In some embodiments of Formula (II), n is 3, and R² and R³ are each methyl (e.g., tripropylene glycol dimethyl ether). In certain embodiments, the polyether agent is dipropylene glycol. It is understood that polypropylene glycols (e.g., dipropylene glycol) can include different isomeric forms. Dipropylene glycol can be present in one or more isomers, 4-oxa-2,6-heptandiol, 4-oxa-1,6-heptandiol, 2-(2-hydroxy-propoxy)-propan-1-ol, and/or 2-(2-hydroxy-1-methyl-ethoxy)-propan-1-ol. In some cases, the dipropylene glycol utilized is a mixture of 4-oxa-2,6-hexandiol and 4-oxa-1,6-hexandiol.

In some embodiments, the polyether agent is of Formula (IIa) and/or (IIb):

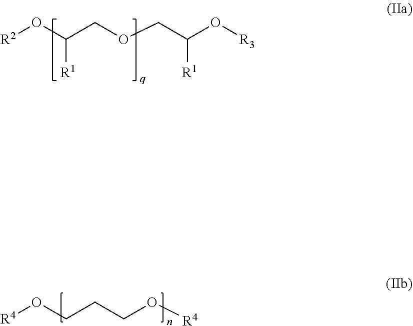

where n is 1-30, q is 1-29; each R¹ is methyl; and R² and R³ and each R⁴ are each independently H, alkyl or a terminal group.

In some embodiments of Formula (IIa), R² and R³ are each H. In some embodiments of Formula (IIa), each R² and R³ are each alkyl, such as C₍₁₋₆₎ alkyl. In some cases, R² and R³ are each methyl. In some embodiments of Formula (IIa), q is 2-29, such as 3-29, 4-29, 5-29, 6-29, 7-29, 8-29, 10-29, or 10-20. In some embodiments of Formula (IIa), q is 1. In some embodiments of Formula (IIa), q is 2-25, such as 2-20, 2-18, 2-16, 2-15, 2-14, 2-13, 2-12, 2-10, 2-8, or 2-6. In some embodiments of Formula (IIb), each R⁴ is H. In some embodiments of Formula (IIb), each R⁴ is alkyl, such as C₍₁₋₆₎ alkyl. In some cases, each R⁴ is methyl. In some embodiments of Formula (II), n is 2-30, such as 3-30, 4-30, 5-30, 6-30, 7-30, 8-30, 10-30, or 10-20. In some embodiments of Formula (IIb), n is 2. In some embodiments of Formula (IIb), n is 2-25, such as 2-20, 2-18, 2-16, 2-15, 2-14, 2-13, 2-12, 2-10, 2-8, or 2-6.

In some embodiments of Formula (IIa), q is 1, and R² and R³ are each H (e.g., dipropylene glycol). In some embodiments of Formula (IIa), q is 2, and R² and R³ are each H (e.g., tripropylene glycol). In some embodiments of Formula (IIa), q is 2, one of R² and R³ is H, and the other of R² and R³ is methyl. In some embodiments of Formula (IIa), q is 2, and R² and R³ are each methyl (e.g., tripropylene glycol dimethyl ether). In some embodiments of Formula (IIb), n is 2, and each R⁴ are each H (e.g., dipropylene glycol). In some embodiments of Formula (IIb), n is 3, and each R⁴ is H (e.g., tripropylene glycol). In some embodiments of Formula (IIb), n is 3, one R⁴ is H, and the other R⁴ is methyl. In some embodiments of Formula (II), n is 3, each R⁴ is methyl (e.g., tripropylene glycol dimethyl ether).

In certain embodiments, the polyether agent is dipropylene glycol. In certain embodiments, the polyether agent is tripropylene glycol. It is understood that polypropylene glycols (e.g., di- or tri-propylene glycol) can include different isomeric forms. Dipropylene glycol can be present in one or more isomers, 4-oxa-2,6-heptandiol, 4-oxa-1,6-heptandiol, 2-(2-hydroxy-propoxy)-propan-1-ol, and/or 2-(2-hydroxy-1-methyl-ethoxy)-propan-1-ol. In some cases, the dipropylene glycol utilized is a mixture of 4-oxa-2,6-hexandiol and 4-oxa-1,6-hexandiol.

In some embodiments of Formula (II)-(IIb), the polyether agent is a (poly)propylene glycol or (poly)propylene glycol ether having a molecular weight in the range of about 120 to 3000. In some embodiments of Formula (II)-(IIb), the polyether has a molecular weight of 3000 or less, such as, 2500 or less, 2000 or less, 1500 or less, or 1000 or less. In some embodiments of Formula (II)-(IIb), the polyether agent has a molecular weight in the range of 100-120, 120-140, 140-160, 160-180, 180-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2200, 2200-2400, 2400-2600, 2600-2800, 2800-3000, 3000-4000, 4000-5000, 5000-6000, 6000-7000, or 7000-8000.

In some embodiments of Formula (II)-(IIb), the polyether agent is PPG 120-160 molecular weight. In some embodiments of Formula (II)-(IIb), the polyether agent is PPG 160-200 molecular weight. In some embodiments of Formula (II)-(IIb), the polyether agent is PPG 200-400 molecular weight. In some embodiments of Formula (II)-(IIb), the polyether agent is PPG 200-600 molecular weight. In some embodiments of Formula (II), the polyether is PPG 3000 molecular weight or less.

(Poly)butylene Glycol Agents

In some embodiments, the polyether agent can be referred to as butylene glycol (n is 1) or (poly)butylene glycol. The polyether agent can be a (poly)-1,4-butylene glycol (R¹ is H) or a (poly)-1,3-butylene glycol (R¹ is methyl). The disclosure provides various sensing solutions including such (poly)butylene glycols for the detection and characterization of a biomolecule in a sample using a nanopore device.

In some embodiments, the polyether agent is of Formula (III):

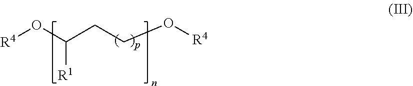

where: p is 1 or 2; n is 1-30; R¹ is H or methyl; and each R⁴ is independently H, alkyl or a terminal group.

In some embodiments of Formula (III), each R⁴ is H. In some embodiments of Formula (III), each R⁴ is alkyl, such as C₍₁₋₆₎ alkyl. In some cases, each R⁴ is methyl. In some embodiments of Formula (III), n is 2-30, such as 3-30, 4-30, 5-30, 6-30, 7-30, 8-30, 10-30, or 10-20. In some embodiments of Formula (III), n is 1. In some embodiments of Formula (III), n is 2-25, such as 2-20, 2-18, 2-16, 2-15, 2-14, 2-13, 2-12, 2-10, 2-8 or 2-6.

In some embodiments of Formula (III), when p is 1, R¹ is methyl. In some embodiments of Formula (III), when p is 2, R¹ is H. In some embodiments of Formula (III), n is 1. In some embodiments of Formula (III), the polyether agent is 1,3-butylene glycol or 1,4-butylene glycol.

In some embodiments of Formula (III), the polyether agent is a (poly)butylene glycol or (poly)butylene glycol ether having a molecular weight in the range of about 120 to 3000. In some embodiments of Formula (III), the polyether has a molecular weight of 3000 or less, such as, 2500 or less, 2000 or less, 1500 or less, or 1000 or less. In some embodiments of Formula (III), the polyether agent has a molecular weight in the range of 100-120, 120-140, 140-160, 160-180, 180-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2200, 2200-2400, 2400-2600, 2600-2800, 2800-3000, 3000-4000, 4000-5000, 5000-6000, 6000-7000, or 7000-8000.

(Poly)alkylene Glycol Ether Agents

As described above, aspects of the polyether agents of formulae (I)-(III) include linear polymers terminated with alkoxy groups. When the polyether agent is a linear polymer, it can be referred to as a (poly)alkylene glycol or (poly)alkylene glycol ether. In some embodiments of formula (I)-(III), the polyether is a (poly)alkylene glycol ether, i.e., where the terminal groups of the polymer are alkyl ether groups. In some embodiments of formula (I)-(III), the polyether is a (poly)alkylene glycol dimethyl ether. In some embodiments of formula (I)-(III), the polyether is a (poly)alkylene glycol diethyl ether. In some embodiments of formula (I), the polyether is a (poly)ethylene glycol dimethyl ether. In some embodiments of formula (II)-(IIb), the polyether is a (poly)propylene glycol dimethyl ether. In some embodiments of formula (III), the polyether is a (poly)1,4-butylene glycol dimethyl ether. In some embodiments of formula (III), the polyether is a (poly)1,3-butylene glycol dimethyl ether.

In certain embodiments, the disclosure provides sensing solution with an effective amount of an acetate, an acrylate, such as poly(ethylene glycol) methyl ether acrylate (CAS 32171-39-4) or the like.

Cation-Salt Agents

The disclosure provides cation-salt agents for the detection and characterization of a biomolecule using a nanopore device. The cation-salt agents of the disclosure are used in sensing solutions at an effective amount to provide enhanced detection and resolution of a biomolecule using a nanopore device. A person skilled in the art would understand that the term “salt agents” can be used interchangeably with the term “electrolytes”.

The disclosure provides various sensing solutions comprising an effective of at least one monovalent cation or monovalent cation salt. In some embodiments, the sensing solution comprises an effective amount of a polyether agent and a monovalent cation. In some embodiments, the monovalent cation can be Li, Na. K, or Cs. In some embodiments, the monovalent cation salt is CsCl, LiCl, NaCl, or KCl. A monovalent cation or a monovalent cation salt can be used in a sensing solution at various molar concentration depending on the biomolecule to be detected. In some embodiments, the monovalent cation or monovalent cation salt can have a total concentration in a sensing solution of about 0.5M, about 1M, about 1.5M, about 2M, about 2.5M, about 3M, about 3.5 M, about 4 M, about 5M, or about 6 M.

The disclosure also provides various sensing solutions comprising an effective of at least one divalent cation or a or divalent cation salt. In some embodiments, the divalent cation can be Ca²⁺ or Mg²⁺. In some embodiments, the divalent cation salt is MgCl₂ or CaCl₂). In some embodiments, the divalent cation or the divalent cation salt can have a total concentration in a sensing solution of about 0.5M, about 1M, about 1.5M, about 2M, about 2.5M, about 3M, about 3.5 M, about 4 M, about 5M, or about 6M

CsCl Agents

The disclosure provides various sensing solutions compositions comprising a CsCl agent for the detection and characterization of a biomolecule using a nanopore device. The CsCl agent can comprise an effective amount in a sensing solution.

The effective amount of a CsCl agent will depend on the biomolecule, method or application used. In some embodiments, an effective amount of CsCl agent is about 0.5, about 1M, about 1.5M, about 2M, about 2.5M, about 3M, about 3.5 M or about 4 M.

The CsCl agents provided by the disclosure can be applied at various concentration in order to form a gradient sensing solution across a membrane in a nanopore device. For example, a higher concentration of CsCl can be applied to the cis chamber and a lower concentration of CsCl can be applied to a trans chamber. Some non-limiting examples include 1M/0.5M CsCl, 2M/1M CsCl, or 3M/1.5M CsCl. In another embodiment, a lower concentration of CsCl can be applied to the cis chamber and a higher concentration of CsCl can be applied to a trans chamber.

CaCl₂ Agents

The disclosure provides various sensing solutions compositions comprising a CaCl₂) agent for the detection and characterization of a biomolecule using a nanopore device. The CaCl₂ agent can comprise an effective amount in a sensing solution.

The effective amount of a CaCl₂) agent will depend on the biomolecule, method or application used. In some embodiments, an effective amount of CaCl₂ agent is about 0.5, about 1M, about 1.5M, about 2M, about 2.5M, about 3M, about 3.5 M or about 4 M.

The CaCl₂ agents provide by the disclosure can be applied as a gradient sensing solution across a membrane of a nanopore device. In some embodiments, a higher concentration of CaCl₂ can be applied to the cis chamber and a lower concentration of CaCl₂) can be applied to a trans chamber. Non-limiting examples of gradient concentrations include 1M/0.5M CaCl₂, 2M/1M CaCl₂), or 3M/1.5M CaCl₂. In other embodiments, a lower concentration of CaCl₂ can be applied to the cis chamber and a higher concentration of CaCl₂ can be applied to a trans chamber.

LiCl Agents

The disclosure provides various sensing solutions compositions comprising a LiCl agent for the detection and characterization of a biomolecule using a nanopore device. The LiCl agent can comprise an effective amount in a sensing solution.

The effective amount of a LiCl agent will depend on the biomolecule, method or application used. In some embodiments, an effective amount of LiCl agent is about 0.5, about 1M, about 1.5M, about 2M, about 2.5M, about 3M, about 3.5 M or about 4 M.

The LiCl agents provided by the disclosure can be applied at various concentration in order to form a gradient sensing solution across a membrane in a nanopore device. For example, a higher concentration of LiCl can be applied to the cis chamber and a lower concentration of LiCl can be applied to a trans chamber. Some non-limiting examples include 1M/0.5M LiCl, 2M/1M LiCl, or 3M/1.5M LiCl. In another embodiment, a lower concentration of LiCl can be applied to the cis chamber and a higher concentration of LiCl can be applied to a trans chamber.

Effective Amount

The effective amount of the polyether agent in a sensing solution will depend on the application, biomolecule, or method used.

Often the effective amount allows for increase accuracy in the detection or characterization of a biomolecule in a nanopore device.

In some embodiments, the effective amount of a polyether agent (e.g., as described herein) in a sensing solution is: about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% v/v. In some embodiments, the effective amount of a polyether agent in a sensing solution is 30% v/v.

In some embodiments, the effective amount of a polyether agent (e.g., as described herein) in a sensing solution is: about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight. In some embodiments, the effective amount of a polyether agent in a sensing solution is 30% or less by weight of a polyether agent (e.g., as described herein).

Like the polyether agent, the effective amount of a CaCl₂, CsCl, or LiCl agent will also depend on the biomolecule, method, or application used.

In some embodiments, the effective amount of a CaCl₂, CsCl, or LiCl agent is about 0.5, about 1M, about 1.5M, about 2M, about 2.5M, about 3M, about 3.5 M or about 4 M. In some embodiments, a combination of any one of these molar concentrations can be used to apply a gradient in an effective amount for the characterization or detection of a biomolecule in a nanopore device.

Additional Agents

A sensing solution can comprise any other agent or chemical known to be in a buffer. As it will be appreciated by one skilled in the art, non-limiting example that can be included in a sensing solution of the disclosure include, buffering solutions, salts, and chelating agents, a carbohydrate, or sugar. It is contemplated that any one of the additional agents can be optimized (e.g., for a concentration) with the sensing solutions using standard screening methods for nanopore detection.

It is contemplated that a divalent or a monovalent cation or a salt can be added to a sensing solution of the disclosure. Non-limited examples of cations or salts that can be added as an additional agent to a sensing solution are: LiCl, NaCl, KCl, MgCl₂, CsCl, CaCl₂, Li, Na, K, Mg, Cs, Ca, or a combination thereof. These salts can be added as various concentrations. For example, an additional salt agent can be used at a molar concentration of greater than 0.01M, 0.02M, 0.05M, 0.1M, 0.2M, 0.5M, 1M, 1.5M, 2M, 2.5M, 3M, 3.5M, 4M, 4.5M, or 5M, or any concentration that works with the sensing solution to increase accuracy.

In some embodiments, the sensing solution of the disclosure can comprise a chelating agent. Chelating agents that can be added to a sensing solution as described herein, include but are not limited to, EDTA, EGTA, or any other chelating agent known in the art. A cheating agent can be added to a sensing solution at different concentrations. For example, the chelating agent can be used at a molar concentration of greater than 0.01M, 0.02M, 0.05M, 0.1M, 0.2M, 0.5M, 1M, 1.5M, 2M, or any concentration that works with the sensing solution to increase accuracy.

A buffer solution can also can be added to a sensing solution of the disclosure. Non-limiting examples of buffer solutions that can be added to a sensing solution are a TRIS-HCl, a Borate, a CHES, a Bis-tris propane, a CAPS, a potassium phosphate, a TRIS, or a HEPES. The buffer solution can be added at various concentrations. For example, the buffer solution can be added to the sensing solution at a molar concentration of greater than 0.01M, 0.02M, 0.05M, 0.1M, 0.2M, 0.5M, 1M, 1.5M, 2M, 2.5M, 3M, 3.5M, 4M, 4.5M, 5M, 6M, 7M, 9M, 10M, 11M, or any concentration that works with the sensing solution to increase accuracy. Also, depending on the application, biomolecule, or method used a sensing solution of the disclosure can also omit certain agents.

In some applications, a sensing solution will not comprise glycerol.

In some applications, a sensing solution will not comprise a PEG greater than 7000. In some applications, a sensing solution provided of the disclosure will not comprise a PEG 8000.

In some embodiments, the PCR amplicon products post-amplification are diluted 1:100 with 4M LiCl+12% PEG 200+10 mM Tris pH 8.8. In some embodiments, the PCR amplifon products are passed through nanopores using 100 mV.

Detection

In some embodiments, the methods of the present disclosure comprise detecting at least two distinct signals corresponding to the first amplification product and the second amplification products based on said detected signal. In some embodiments, the distinct signal is detected using a nanopore device.

In some embodiments, the signals from said first and second genetic variants are discriminated by a characteristic selected from the group consisting of: amplicon length, sequence, physical or chemical modification incorporated into the primer, and physical or chemical probe added to the amplicon post-amplification.

In some embodiments, the physical or chemical probe comprises PEG. In some embodiments, the physical or chemical probe comprises a fluorophore. In some embodiments, the PEG or fluorophore is bound to DNA, LNA, XNA, or PNA. In some embodiments, the amplification reaction comprises one or more modified nucleotides or one or more modified primers. In some embodiments, the modification comprises a direct label or an indirect label. In some embodiments, the modification comprises a charged chemical moiety, a neutral chemical moiety, a hydrophobic moiety, or a hydrophilic moiety. In some embodiments, the modification comprises a fluorescent dye.

### Probes and Voltage-Sensitive Moieties

Depending on the application, the methods can include the use of a molecular probe. The use of probes to enhance detection using a nanopore device is described in U.S. application Ser. No. 15/513,472, which is herein incorporated by reference.

Depending of molecule and probe type it may be desirable to attach or link the probe prior to analysis. The attachment of the probe to the molecule prior to analysis can be externally (outside of the device) or in the nanopore device, but before analysis.

It will be understood by those skilled in the art that various methods well known in the art can be used to attach the probe to the molecule, including but not limited to, hybridization, conjugation, linkage chemistry, and by various chemical bonds (e.g., covalent, hydrogen and the like).

Probes

Probes are capable of specifically binding to a site on a molecule to be detected or characterized. Often binding site of the probe can be a sequence, a modification, or a structure to be detected or characterized.

Examples of probe molecules that can be used with the disclosure, include but are not limited to, a single-strand DNA, a PNA (protein nucleic acid), bis-PNA, gamma-PNA, a PNA-conjugate that increases size or charge of PNA. Other examples of probe molecules are from the group consisting of a natural or recombinant protein, protein fusion, DNA binding domain of a protein, peptide, a nucleic acid, oligo nucleotide, TALEN, CRISPR, a PNA (protein nucleic acid), bis-PNA, gamma-PNA, a PNA-conjugate that increases size, charge, fluorescence, or functionality (e.g. oligo labeled), or any other PNA derivatized polymer, and a chemical compound.

In some aspects, the probe comprises a γ-PNA. γ-PNA has a simple modification in a peptide-like backbone, specifically at the γ-position of the N-(2-aminoethyl)glycine backbone, thus generating a chiral center (Rapireddy S., et al., 2007. J. Am. Chem. Soc., 129:15596-600; He G, et al., 2009, J. Am. Chem. Soc., 131:12088-90; Chema V, et al., 2008, Chembiochem 9:2388-91; Dragulescu-Andrasi, A., et al., 2006, J. Am. Chem. Soc., 128:10258-10267). Unlike bis-PNA, γ-PNA can bind to dsDNA without sequence limitation, leaving one of the two DNA strands accessible for further hybridization.

In some aspects, the function of the probe is to hybridize to a polynucleotide with a target sequence by complement base pairing to form a stable complex. The PNA molecule may additionally be bound to additional molecules to form a complex has sufficiently large cross-section surface area to produce a detectable change or contrast in signal amplitude over that of the background, which is the mean or average signal amplitude corresponding to sections of non-probe-bound polynucleotide.

The stability of the binding of the polynucleotide target sequence to the PNA molecule is important in order for it to be detected by a nanopore device. The binding stability must be maintained throughout the period that the target-bearing polynucleotide is being translocated through the nanopore. If the stability is weak, or unstable, the probe can separate from the target polynucleotide and will not be detected as the target-bearing polynucleotide threads through the nanopores.

In a particular embodiment, an example of a probe is a PNA-conjugate in which the PNA portion specifically recognizes a nucleotide sequence and the conjugate portion increases the size/shape/charge differences between different PNA-conjugates.

Moieties

Different reactive moieties may be incorporated into the ligands to provide chemical handle to which labels maybe conjugated. Examples of reactive moieties include, but are not limited to, primary amines, carboxylic acids, ketones, amides, aldehydes, boronic acids, hydrazones, thiols, maleimides, alcohols, and hydroxyl groups, and biotin.

In some embodiments, a PNA ligand can be modified as to increase ligand charge, and therefore facilitate detection by a nanopore. Specifically, this ligand, which binds to the target DNA sequence by complementary base pairing and Hoogsteen base pairing between the bases on the PNA molecule and the bases in the target DNA, has cysteine residues incorporated into the backbone, which provide a free thiol chemical handle for labeling. Here, the cysteine is labeled to a peptide 2-aminoethylmethanethiosulfonate (MTSEA) through a maleimide linker, which provides a means to detect whether the ligand is bound to its target sequence since the label/peptide gives an increase to the ligand charge. This greater charge results in a greater change in current flow through the pore compared to an unlabeled PNA.

In some aspects, to increase the contrast in the change between the ligand bound polynucleotide and other background molecules present in the sample, modification can be made to the pseudo-peptide backbone to change the overall size of the ligand (e.g., PNA) to increase the contrast.

PEG Probes

In addition, small particle, molecules, protein, peptides, or polymers (e.g. PEG) can be conjugated to the pseudo-peptide backbone to enhance the bulk or cross-sectional surface area of the ligand and target-bearing polynucleotide complex. Enhanced bulk serves to improve the signal amplitude contrast so that any differential signal resulting from the increased bulk can be easily detected. Examples of small particle, molecules, protein, or peptides can be conjugated to the pseudo-peptide backbone include but are not limited to alpha-helical forming peptides, nanometer-sized gold particles or rods (e.g. 3 nm), quantum dots, polyethylene glycol (PEG). Method of conjugation of molecules are well known in the art, e.g. in U.S. Pat. Nos. 5,180,816, 6,423,685, 6,706,252, 6,884,780, and 7,022,673, which are hereby incorporated by reference in their entirety.

The embodiments above describe PEG labeling is through cysteine residues, however other residues can also be used. For example, Lysine residue are easily interchanged with cysteine residues to enable linkage chemistry using NHS-esters and free amines. Also, PEG can easily be interchanged with other bulk-adding constituents, like Dendrons, beads, or rods. between the bifunctional linker and the PNA, or to directly couple the Dendron. Someone skilled in the art would recognize the flexibility of this system in that the amino acid can be changed and linkage chemistry modified for that particular amino acid, e.g. Serine reactive isocyanates. Some examples of linkage chemistry that can be used for this reaction is listed in the table below.

Nanopore Device

Aspects of the present disclosure comprise detecting at least two distinct signals corresponding to the first amplification product and second amplification product. In some embodiments, the distinct signals are detected using a nanopore device. In some embodiments, the detection is performed using a sensor configured to measures an electrical signal that fluctuates upon translocation of the amplification product through a nanopore. In some embodiments, the electrical signal is distinct between said first and second amplification products.

Any nanopore device can be used with the methods as disclosed herein. Examples of devices that can be used with the disclosure included but are not limited to a solid-state nanopore device, a biological nanopore device or a hybrid nanopore device.

The methods of the disclosure can use any of the devices or membranes known in the art.

Upon reading the disclosure a skilled artisan will choose an appropriate nanopore device and membrane. In some applications, the device can have a pore diameter size greater than about 20 nm, about 25 nm, or about 30 nm. In other applications, the device can have a pore diameter size greater than about 60 nm, about 70 nm, about 80 nm, about 90 nm, about 100 nm, about 110 nm, or about 120 nm.

A nanopore device can include at least a pore that forms an opening in a structure separating an interior space of the device into two volumes, and at least a sensor configured to identify objects (for example, by detecting changes in parameters indicative of objects) passing through the pore. A device used with the disclosure can have a pore of any architecture (e.g., round shape, funnel shape, etc.). A device used with the disclosure can have a single pore, a dual pore, or it can have several pores, such as, for example, an array of pores.

Nanopore devices used for the methods described herein are also disclosed in PCT Publication No. WO/2013/012881; U.S. Pat. Nos. 10,344,327, 9,863,912, 10,208,342, 10,048,245; and U.S. Patent Application Publication Nos. 20190383806, 20180023115, and 20190250143, each of which are incorporated herein by reference in their entirety.

In some embodiments, a nanopore device includes a membrane separating two volumes or chambers, where the membrane has a nanopore through the membrane that allows fluid communication between the two volumes. The nonporous membrane can be made from a biological substrate (e.g., lipid membrane) or a non-biological substrate (e.g., solid substrate) or any other substrate known in the art. In some embodiments, the nanopore device can be a solid-state nanopore device, biological nanopore device or a hybrid nanopore device.

When the two volumes contain an electrolyte, such as a salt, a current can flow through the pore by applying a voltage potential across the pore, e.g., via electrodes on either side of the pore. Using a low-noise transimpedance amplifier, nanopore devices monitor ionic current through a single pore that separates two chambers or volumes.

Voltage is applied across the membrane, creating a current (e.g., ionic current) through the nanopore that is filtered, sampled, and recorded for analysis. When the voltage captures a single molecule such as DNA, RNA or protein, it passes through the pore and temporarily shifts the current, creating a single molecule “event.” Using a variety of techniques as described for example in US Patent Application Publication No. 2016/0266089, “Target Detection with Nanopore,” incorporated herein by reference in its entirety, one can detect and quantitate the presence of a specific target molecule from among a population of background molecules by analyzing the distribution of recorded events.

Also provided herein, is a nanopore device comprising a layer that separates an interior space of the device into the first volume and a second volume, wherein said layer comprises a nanopore; wherein said first and second volume are in fluidic communication through said nanopore, and wherein said first volume or said second volume comprises a buffer comprising ethylene glycol. In some embodiments, the system further comprises a first electrode in said first volume and a second electrode in said second volume, wherein said first and second electrode are configured to apply a voltage potential across said nanopore. In some embodiments, the system further comprises a target biomolecule in said first volume or said second volume, wherein said voltage potential induces translocation of said target biomolecule through said nanopore.

The pore(s) in the nanopore device are of a nano scale or micro scale. In one aspect, each pore has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In some embodiments, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In some embodiments, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some embodiments, the nanopore device further includes means to move an amplicon product post-amplification to identify objects that pass through the pore.

In some embodiments, the nanopore device includes a plurality of chambers, each chamber in communication with an adjacent chamber through at least one pore. Among these pores, two pores, namely a first pore and a second pore, are placed so as to allow at least a portion of a target polynucleotide to move out of the first pore and into the second pore. Further, the device includes a sensor at each pore capable of identifying the target polynucleotide during the movement. In some embodiments, the identification entails identifying individual components of the target polynucleotide. In another aspect, the identification entails identifying payload molecules bound to the target polynucleotide. When a single sensor is employed, the single sensor may include two electrodes placed at both ends of a pore to measure an ionic current across the pore. In another embodiment, the single sensor comprises a component other than electrodes.

In some embodiments, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter. In some embodiments, each pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In some embodiments, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some embodiments, the pore has a substantially round shape. “Substantially round”, as used here, refers to a shape that is at least about 80 or 90% in the form of a cylinder. In some embodiments, the pore is square, rectangular, triangular, oval, or hexangular in shape.

In some embodiments, the pore has a depth that is between about 1 nm and about 10,000 nm, or alternatively, between about 2 nm and about 9,000 nm, or between about 3 nm and about 8,000 nm, etc.

In some embodiments, the nanopore extends through a membrane. For example, the pore may be a protein channel inserted in a lipid bilayer membrane or it may be engineered by drilling, etching, or otherwise forming the pore through a solid-state substrate such as silicon dioxide, silicon nitride, grapheme, or layers formed of combinations of these or other materials. Nanopores are sized to permit passage through the pore of the scaffold:fusion:payload, or the product of this molecule following enzyme activity. In other embodiments, temporary blockage of the pore may be desirable for discrimination of molecule types.

In some embodiments, the length or depth of the nanopore is sufficiently large so as to form a channel connecting two otherwise separate volumes. In some such aspects, the depth of each pore is greater than 100 nm, 200 nm, 300 nm, 400 nm, 500 nm, 600 nm, 700 nm, 800 nm, or 900 nm. In some aspects, the depth of each pore is no more than 2000 nm or 1000 nm.

In some embodiments, the device has electrodes in the chambers connected to one or more power supplies. In some aspects, the power supply includes a voltage-clamp or a patch-clamp, which can supply a voltage across each pore and measure the current through each pore independently. In this respect, the power supply and the electrode configuration can set the middle chamber to a common ground for both power supplies. In one aspect, the power supply or supplies are configured to apply a first voltage V₁ between the upper chamber (Chamber A) and the middle chamber (Chamber B), and a second voltage V₂ between the middle chamber and the lower chamber (Chamber C).

In some aspects, the first voltage V₁ and the second voltage V₂ are independently adjustable. In one aspect, the middle chamber is adjusted to be a ground relative to the two voltages. In one aspect, the middle chamber comprises a medium for providing conductance between each of the pores and the electrode in the middle chamber. In one aspect, the middle chamber includes a medium for providing a resistance between each of the pores and the electrode in the middle chamber. Keeping such a resistance sufficiently small relative to the nanopore resistances is useful for decoupling the two voltages and currents across the pores, which is helpful for the independent adjustment of the voltages.

Adjustment of the voltages can be used to control the movement of charged particles in the chambers. For instance, when both voltages are set in the same polarity, a properly charged particle can be moved from the upper chamber to the middle chamber and to the lower chamber, or the other way around, sequentially. In some aspects, when the two voltages are set to opposite polarity, a charged particle can be moved from either the upper or the lower chamber to the middle chamber and kept there.

The adjustment of the voltages in the device can be particularly useful for controlling the movement of a large molecule, such as a charged polymer scaffold, that is long enough to cross both pores at the same time. In such an aspect, the direction and the speed of the movement of the molecule can be controlled by the relative magnitude and polarity of the voltages as described below.

In some embodiments, the method comprises applying 100 mV bias to the nanopore chip (e.g. trans side positive) using a voltage-clamp amplifier. In some embodiments, the method comprises recording the ionic current data. In some embodiments, the ionic current data was recorded using custom software at a sampling rate of 125 kHz for approximately 5 minutes, or enough time to collect ˜1000 molecular translocation events for each reagent. In some embodiments, the method comprises recording each sample % Trait-Extract on 4 independent pores.

In some embodiments, the nanopore diameters range in size from 25-41 nm.

The device can contain materials suitable for holding liquid samples, in particular, biological samples, and/or materials suitable for nanofabrication. In one aspect, such materials include dielectric materials such as, but not limited to, silicon, silicon nitride, silicon dioxide, graphene, carbon nanotubes, TiO₂, HfO₂, Al₂O₃, or other metallic layers, or any combination of these materials. In some aspects, for example, a single sheet of graphene membrane of about 0.3 nm thick can be used as the pore-bearing membrane.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of molecular biology, protein chemistry, biochemistry, and recombinant DNA techniques, within the skill of the art.

### Example 1: Three-Primer Amplification and Fluorescence Detection

An assay for the determination of percentage by weight of Roundup Ready MON04032 (RR) seeds in a mixed population with conventional soybean seeds was performed. This assay uses a difference in PCR length to separate the two PCR products by capillary electrophoresis and measures the fluorescence from an intercalating dye. The fluorescence can then be used to directly calculate the percentage by weight in the starting sample. Alternatively, the number of DNA molecules estimated from the fluorescence can be used for the calculation.

Target Genes, PCR Primers and Amplicons

Target genes representing the wildtype (SEQ ID NO: 1) and the mutant variant indicating RR seeds (SEQ ID NO: 2) are shown below. In bold and underlined are target sequences for the primers used in the PCR amplification reaction for each.

SEQ ID NO: 1 >Wildtype Glycine max chromosome 2 pos 8001961..8002760
(SEQ ID NO: 1)
ACACTAAATGCATGTTTAGATTAAAGCTTGTAAATTTAAGTTTGAGTTAAATTTA
AGTTAGTAAATTGAGTTTGCAAAAATAACTTAGTGTACTACGAAATTGAGTAGT
TTTCAGACAAAATTTTAGTTGCACATAGTTTTGAGGATAACCAAACTATGTTTAG
CATTCAAAAGTACTTTTTGAACAGTTAACCAAACATGTCCTAAATCATTATAC
ATTAACAAATTCCTTTATTAAAAAAAGTTTAAATATGATTTATAATGTTCATAGT
ATTAAGTTCTGGATTATTAGTTTTTAAAACATTTGATCTATAAGGTTAGTTTTATC
AAGCGGCAAGTCAATCGTGTCGTTCACATCTTTGCAAGAGCACCTCGTTTCTATG
CTAATTACTGCATTTTTTTTATTTTAACCTGTATGTATGATCTTATTTTGAATGAA
ATGCAATAAGTTATTTCTAGTAAAAAAAAATAAACATTTGATAGAAACAAATTA
AAGCATGCAAAAATAACTCATTAGCATCGGTTAAATTGAAGGGTTTGAATAAT
TTGCACAAGGTTCTGAATTCAAATCTTGTTCATTGTAAAAAATAAAGCATGAAA
AAAAGAGGGGCAAAATTTAAACATAAATAATAAGGATTCGGTAAGATCGAGAA
TCGCAATGTAGGGATTCAGATAAAAATATGTTAAGCAGATTGAAGGATAATATA
TATATATATATATATATATATATATATATATATTGTATCTGAAGGATAATATTTT
AAATTTACTGAAGCATAGCTCCAAAATTACGCGGTTTC
SEQ ID NO: 2 >Mutant MON04032 transgene/chromosome junction B
(SEQ ID NO 2)
TTGAAGATTTAGGAACTTGGGGTTTATGGAAATTGGAATTGGGATTAAGGGTTT
GTATCCCTTGTGCCATGTTGTTAATTTGTGCCATTCTTGAAAGATCTGCTAGAGT
CAGCTTGTCAGCGTGTCCTCTCCAAATGAAATGAACTTCCTTATATAGAGGAAG
GGTCTTGCGAAGGATAGTGGGATTGTGCGTCATCCCTTACGTCAGTGGAGATAT
CACATCAATCCACTTGCTTTGAAGACGTGGTTGGAACGTCTTCTTTTTCCACGAT
GCTCCTCGTGGGTGGGGGTCCATCTTTGGGACCACTGTCGGCAGAGGCATCTTC
AACGATGGCCTTTCCTTTATCGCAATGATGGCATTTGTAGGAGCCACCTTCCTT
TTCCATTTGGGTTCCCTATGTTTATTTTAACCTGTATGTATGATCTTATTTTGAAT
GAAATGCAATAAGTTATTTCTAGTAAAAAAAAATAAACATTTGATAGAAACAAA
TTAAAGCATGCAAAAATAACTCATTAGCATCGGTTAAATTGAAGGGTTTGAAT
AATTTGCACAAGGTTCTGAATTCAAATCTTGTTCATTGTAAAAAATAAAGCATG
AAAAAAAGAGGGGCAAAATTTAAACATAAATAATAAGGATTCGGTAAGATCGA
GAATCGCAATGTAGGGATTCAGATAAAAATATGTTAAGCAGATTGAAGGATAAT
ATATATATATATATATATATATATATATATATATATTGTATCTGAAGGATAATAT
TTTAAATTTACTGAAGCATAGCTCCAAAATTACGCGGTTTC

Primers used in the reaction are provided below. The Fc primer is common to the amplification reaction for both targets, while the Rw primer is designed to generate the wildtype PCR product (i.e., amplicon), and the Rm primer is designed to generate the mutant/variant PCR product. For this reaction, the mutant PCR product is 222 bp in length, and the wildtype PCR product is 356 bp in length.

Primers

Rw = MDP_AAPY_MON04032 F
(SEQ ID NO: 3)
CAGTTAACCAAACATGTCCTAAATC
Rm = MDP_AAPK_MON04032 F
(SEQ ID NO: 4)
CATCTTCAACGATGGCCTTTC
Fc = MDP_AAPJ_MON04032 R
(SEQ ID NO: 5)
TCAAACCCTTCAATTTAACCGA

DNA Extraction

35 g of w/w ‘Gamekeeper RR’/‘Black Jet’ whole soybean mixes were Dry blended in an Oster blender for 10 seconds, then 100 ml of 250 mM NaOH was added and them mixture shaken for 15 seconds. The liquid was passed through a coffee filter, and then diluted 1:1000 with water. This diluted sample was used directly as the template for PCR.

PCR Amplification

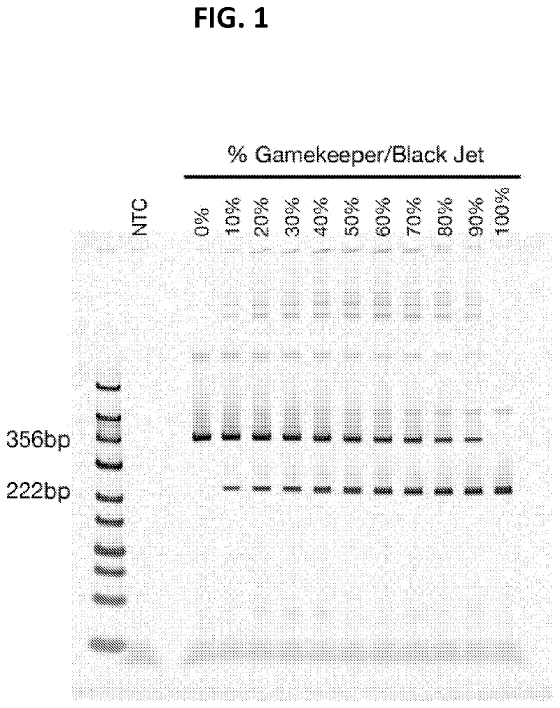

For each series of experiments, a master-mix was made with dNTPs (New England Biolabs), Phusion HF Buffer (New England Biolabs), Phusion Hot Start Flex DNA Polymerase (New England Biolabs), DNA oligonucleotide primers (Integrated DNA Technologies), and water in a 1.5 mL Eppendorf tube. All tubes were kept on ice. 21 μL of the master-mix was mixed in with 4 μL of 1:1000 soybean extract by gently pipetting up and down in a thin walled 200 μL PCR tube. To reduce the effects of random errors arising during any single PCR, multiple PCRs may be performed from a single test template, and then mixed together before analysis. All amplifications were performed in a Bio-Rad C1000 thermal cycler. PCR amplification was performed using the reagents described in Table 1 and the PCR cycling conditions shown in Table 2. After cycling, 3 μL of the PCR product was run on a 6% TBE PAGE gel at 200V for 25 minutes and post-stained with SYBR Green, to quality check the reactions (FIG. 1)

TABLE 1
PCR Master-mix

| | Stock Conc | Final Conc | Volume |
|---|---|---|---|
| Soybean | 1:1000 | 1:6250 | 4 μL |
| Extract | | | |
| Primer Fc | 30 μM | 0.6 μM | 0.5 μL |
| Primer Rw | 30 μM | 0.6 μM | 0.5 μL |
| Primer Rm | 30 μM | 0.6 μM | 0.5 μL |
| dNTPs | 10 μM | 200 μM | 0.5 μL |
| HF PCR | 5x | 1x | 5 μL |
| Buffer | | | |
| HS Phusion | 2 U/μL | 0.5 U | 0.25 μL |
| Water | | | 13.75 μL |
| Total | | | 25 μL |

TABLE 2
PCR Cycling Conditions

| temp | time | cycles | ramp |
|---|---|---|---|
| 95° C. | 0:30 | ×1 | 3°/sec |
| 95° C. | 0:05 | ×35 | 3°/sec |
| 55° C. | 0:10 | | |
| 75° C. | 0:10 | | |
| 72° C. | 0:30 | ×1 | 3°/sec |
| 4° C. | hold | | 3°/sec |

PCR products were analyzed using an Agilent Fragment Analyzer capillary electrophoresis device with dsDNA Reagent Kit (35-1500 bp) (DNF-910), as per the instructions. Each of the two PCR products (PCRw and PCRm) were recorded as both percentage of total fluorescence (% fluorescence) (% fluorescenceW+% fluorescenceM=100%), and percentage of total molecules (% molecules) as calculated from the fluorescence and length (% moleculesW+% moleculesM=100%).

Data Analysis—Generating a Correction Equation

The capillary electrophoresis data (across the entire range of interest) from several samples with known percentage by weight of Mutant RR soybeans (% weightM) was input into Microsoft Excel software (any graphing software can be used), and a graph generated with the % fluorescenceM or % moleculesM of the PCRm product band on the x-axis, vs the % weightM of the Mutant RR soybeans used to make the DNA template on the y-axis. From these graphs, polynomial trendlines were calculated using the software. These equations are then used as correction equations to calculate % weightM from % fluorescenceM (or % moleculesM) for unknown data. Alternatively, trendlines could be calculated for the wildtype PCRw with % weightW, % fluorescenceW, and % moleculesW. As long as the assay is performed by following the same protocol, the correction equation can be improved with additional data, with repeated experiments at fine intervals generating the most accurate correction equation.

To generate the correction equations to calculate the percent by weight of Mutant RR soybeans from % moleculesM or % fluorescenceM, control samples with 0%, 10%, 30%, 50%, 70%, 90% and 100% weight of Mutant RR soybeans in the sample were amplified and detected as described above. Resulting % moleculesM and % fluorescenceM are shown in Table 3.

TABLE 3
Training Data
Training Data

| % weightM | % moleculesM | % fluorescenceM |
|---|---|---|
| 0 | 0.0 | 0.0 |
| 10 | 4.9 | 9.1 |
| 30 | 16.5 | 27.8 |
| 50 | 34.7 | 50.9 |
| 70 | 48.8 | 65.0 |
| 90 | 80.1 | 88.7 |
| 100 | 100.0 | 100.0 |

Correction Equations

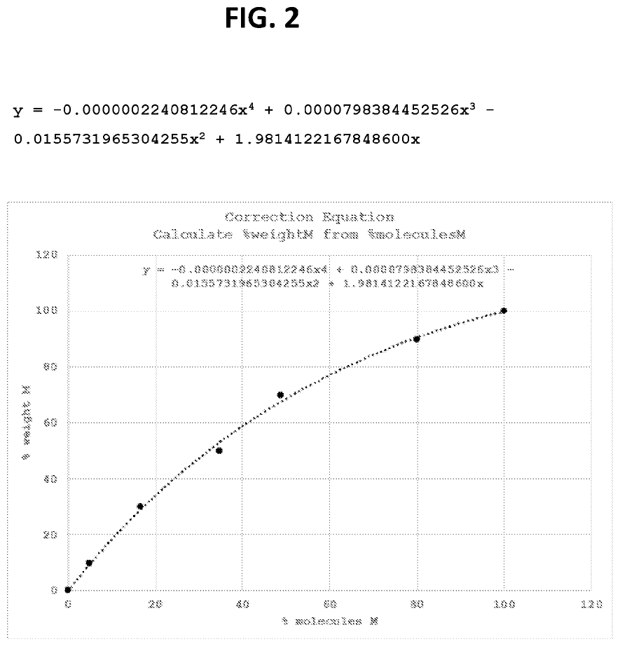

Based on the above data, a plot of % weight (y-axis) vs % moleculesM (x-axis) was generated (FIG. 2) and a curve was fit with the following equation:

y=−0.0000002240812246x⁴+0.0000798384452526x³−0.0155731965304255x²+1.9814122167848600x

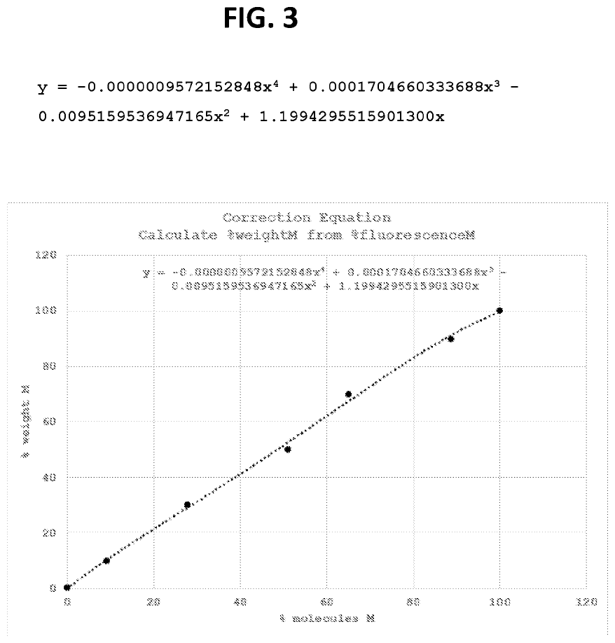

Based on the above data, a plot of % weight (y-axis) vs % fluorescenceM (x-axis) was generated (FIG. 3) and a curve was fit with the following equation:

y=−0.0000009572152848x⁴+0.0001704660333688x³−0.0095159536947165x²+1.1994295515901300x

Data Analysis—Test Data

The raw data from the capillary electrophoresis analysis was used as input into the correction equations to calculate the starting percentage by weight from the test samples. Results are shown in Table 4 and Table 5.

TABLE 4
Determining % weight from % moleculesM
Test Data 1

| % weightM | % moleculesM | calc % weightM | % error |
|---|---|---|---|
| 20 | 11.0 | 20.0 | 0.0 |
| 40 | 24.0 | 39.6 | 0.4 |
| 60 | 44.0 | 63.0 | −3.0 |
| 80 | 66.7 | 82.1 | −2.1 |

TABLE 5
Determining % weight from % fluorescenceM
Test Data 2

| | | calculated | |
|---|---|---|---|
| % weightM | % fluorescenceM | % weightM | % error |
| 20 | 19.4 | 20.8 | −0.8 |
| 40 | 38.1 | 39.3 | 0.7 |
| 60 | 60.5 | 62.7 | −2.7 |
| 80 | 79.6 | 82.7 | −2.7 |

The calculated % weightM was highly accurate with a % error of 3% or less for this example using the method described herein.

### Example 2: Three-Primer Amplification and Nanopore Detection

An assay for the determination of percentage by weight of Roundup Ready MON04032 (RR) seeds in a mixed population with conventional soybean seeds. This assay uses a difference in PCR length to distinguish the two PCR products using the characteristics of the electrical signal as the molecules pass through a solid-state nanopore. Those signals can then be used to directly calculate the percentage by weight in the starting sample.

Target Genes, PCR Primers and Amplicons

Target genes representing the wildtype (SEQ ID NO: 6) and the mutant variant indicating RR seeds (SEQ ID NO: 7) are shown below. In bold and underlined are target sequences for the primers used in the PCR amplification reaction for each.

SEQ ID NO: 6 >Wildtype Glycine max chromosome 2 pos 7841570..78423692
(SEQ ID NO: 6)
AAGTCCCCATAGATTACATAACCGACAAAAACAATGCCCATATCTAGGAAGCCA
ATACAGTCGATATAAATAACATTAATCCACACCTAAATGTCATAACTCATAAAC
AACCCTAAGCATTAAATTGGAGTCCAAGTACTAGAGAAAGGCTTAATTTCGTAT
TGTAATCTCCCTCAGAATTTCTTAATCTTGTGATCAACAAAGCATATCCTCGTTTT
AAATTCTAAAGGTTATGGCAAAATTCACTGGCATACGAACAATTCATATATCCA
TTCCTATTATATATAGTTGGCAGAAGTACAAGGAGGCGCCAAATAGAAAACACA
AATTGGAACGGTGAAGAGAAAGAAGAGTACCTCGGAGAGAGTTGAGGCGAGA
GATGAGATCGGGAGGGAAGAGATTGGGATCGGAGAAGAACTGTTTGAGGCGAA
TGGCCTGGTCGTCGCGGCCATCGTCGAGAAGTTCGTGAAGAAGCTCGAATGCG
GTGAGAAGGTAGTTCTCTTCCAACAGAAAGTTCACCACGCAATTGCACAGCGA
AGATCTCTCCACGTCCATTTTCTCTCTCTGTCTCTGATCTTAAGCCATTCATTCAA
GACAAGACAAGAGAAGAGAAGAGAAGAGAAGAGAACACTCTCAGTCAGATCGT
GGTTTCAACTTTCAAGACTGTGCTAGCTAGTTAGGTGCCATCTTACATGTTTACT
TTTTTTCTTTATAAGATTAAATTGCTGAATACCATGCTCTCCTGTGTCCAAAGCA
GTACACCCGCGTAAAAATAGATTTCATCGTCCTTTCGATTTTAC
SEQ ID NO: 7 >Mutant MON04032 transgene/chromosome junction A
(SEQ ID NO: 7)
AATTAAATAAATCAATTACTTCATAAATAATTTTTTTTATAGAATATGTTGACAT
TCTAGCCGGATATAGAACTAATGTAAAGAAACCTTAAAAATTTTGTTTGGAAGA
ATATGTTATTGAAAGACAAATCTAATTAAGTTTATCAGGGTCATTTGTTGAAGAT
AGGAAACCTTCAGCAATTTGAATATTAAGTAACTGCTTCTCCCAGAATGATCG
GAGTTTCTCCTCCTGCTATTACATGAGCAAAAATAAAAAATAAATAAAAGATA
AGATTAAGCTTCAACATGTGAAGGAGTAGTACACTCACCAGTGACCCTAATAG
GCAACAGCATGAAAAAAAATAAAAAAGAATAAAAATAGCATCTACATATAGC
TTCTCGTTGTTAGAAAAACAAAACTATTTGGGATCGGAGAAGAACTGTTTGAG
GCGAATGGCCTGGTCGTCGCGGCCATCGTCGAGAAGTTCGTGAAGAAGCTCGAA
TGCGGTGAGAAGGTAGTTCTCTTCCAACAGAAAGTTCACCACGCAATTGCACA
GCGAAGATCTCTCCACGTCCATTTTCTCTCTCTGTCTCTGATCTTAAGCCATTCAT
TCAAGACAAGACAAGAGAAGAGAAGAGAAGAGAAGAGAACACTCTCAGTCAG
ATCGTGGTTTCAACTTTCAAGACTGTGCTAGCTAGTTAGGTGCCATCTTACATGT
TTACTTTTTTTCTTTATAAGATTAAATTGCTGAATACCATGCTCTCCTGTGTCCAA
AGCAGTACACCCGCGTAAAAATAGATTTCATCGTCCTTTCGATTTTAC

Primers used in the reaction are provided below. The Fc primer is common to the amplification reaction for both targets, while the Rw primer is designed to generate the wildtype PCR product (i.e., amplicon), and the Rm primer is designed to generate the mutant/variant PCR product. For this reaction, the mutant PCR product is 298 bp in length, and the wildtype PCR product is 153 bp in length.

Primers

Rw = MDP_AAPM_MON04032 F
(SEQ ID NO: 8)
AAGAAGAGTACCTCGGAGAGAG
Rm = MDP_AAOO_MON04032 F
(SEQ ID NO: 9)
CTCCCAGAATGATCGGAGTTTC
Fc = MDP_AAOP_MON04032 R
(SEQ ID NO: 10)
AACTACCTTCTCACCGCATTC

DNA Extraction

35 g of w/w ‘Gamekeeper RR’/‘Black Jet’ whole soybean mixes were Dry blended in an Oster blender for 10 seconds, then 100 ml of 250 mM NaOH was added and them mixture shaken for 15 seconds. The liquid was passed through a coffee filter, and then diluted 1:1000 with water. This diluted sample was used directly as the template for PCR.

PCR Amplification

For each series of experiments, a master-mix was made with dNTPs (New England Biolabs), Phusion HF Buffer (New England Biolabs), Phusion Hot Start Flex DNA Polymerase (New England Biolabs), DNA oligonucleotide primers (Integrated DNA Technologies), and water in a 1.5 mL Eppendorf tube. All tubes were kept on ice. 21 μL of the master-mix was mixed in with 4 μL of 1:1000 soybean extract by gently pipetting up and down in a thin walled 200 μL PCR tube. To reduce the effects of random errors arising during any single PCR, multiple PCRs may be performed from a single test template, and then mixed together before analysis. All amplifications were performed in a Bio-Rad C1000 thermal cycler. After cycling, 3 μL of the PCR product was run on a 6% TBE PAGE gel at 200V for 25 minutes and post-stained with SYBR Green, to quality check the reactions.

TABLE 6
PCR Master-mix

| | Stock Conc | Final Conc | Volume |
|---|---|---|---|
| Soybean | 1:1000 | 1:6250 | 4 μL |
| Extract | | | |
| Primer Fc | 30 μM | 0.6 μM | 0.5 μL |
| Primer Rw | 30 μM | 0.6 μM | 0.5 μL |
| Primer Rm | 30 μM | 0.6 μM | 0.5 μL |
| dNTPs | 10 μM | 200 μM | 0.5 μL |
| HF PCR | 5x | 1x | 5 μL |
| Buffer | | | |
| HS Phusion | 2 U/μL | 0.5 U | 0.25 μL |
| Water | | | 13.75 μL |
| Total | | | 25 μL |

TABLE 7
PCR Cycling Conditions

| temp | time | cycles | ramp |
|---|---|---|---|
| 95° C. | 0:30 | ×1 | 3°/sec |
| 95° C. | 0:05 | ×35 | 3°/sec |
| 55° C. | 0:10 | | |
| 75° C. | 0:10 | | |
| 72° C. | 0:30 | ×1 | 3°/sec |
| 4° C. | hold | | 3°/sec |

Analysis without Separation of PCR Products—Solid-State Nanopore

PCR products were analyzed using solid-state silicon-based nanopores with a diameter of ˜30 nm. The PCR products were diluted 1:100 with 4M LiCl+12% PEG 200+10 mM Tris pH 8.8 and passed through nanopores using 100 mV. For each sample, 500-1000 temporary shifts in current (events) associated with the translocation of DNA were recorded.

Data Analysis—Generating a Correction Equation

Events were classified into two populations using characteristics of the event profiles obtained from samples containing only one of the two lengths of DNA (either 100% wildtype PCRw, or 100% mutant PCRm). Using the classification criteria obtained from these experiments, all of the events from several samples with known percentage by weight of Mutant RR soybeans (% weightM) were analyzed, and each event placed into one of the two categories. Each of the two PCR products (PCRw and PCRm) were recorded as the percentage of total events (% events) (% eventsW+% eventsM=100%). Based on the analysis of the pure populations, the percentage of events that are misclassified (the false positive and false negative events) was also determined.

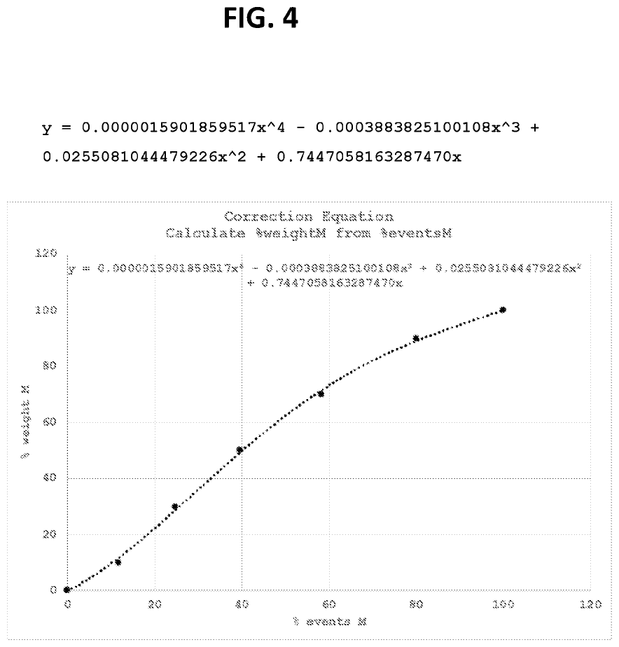

The nanopore event data (corrected for the false positive and false negative rates) from three independent nanopores (across the entire range of interest) from several samples with known percentage by weight of Mutant RR soybeans (% weightM) was averaged (Table 8), and input into Microsoft Excel software (any graphing software can be used), and a graph generated with the % eventsM of the PCRm product band on the x-axis, vs the % weightM of the Mutant RR soybeans used to make the DNA template on the y-axis. From these graphs, polynomial trendlines were calculated using the software (FIG. 4). These equations are then used as correction equations to calculate % weightM from % eventsM for unknown data (Table 9). Alternatively, trendlines could be calculated for the wildtype PCRw with % weightW and % eventsW. As long as the assay is performed by following the same protocol, the correction equation can be improved with additional data, with repeated experiments at fine intervals generating the most accurate correction equation.

TABLE 8
Training Data
Training Data

| % weightM | % moleculesM |
|---|---|
| 0 | 0.0 |
| 10 | 11.8 |
| 30 | 24.7 |
| 50 | 39.6 |
| 70 | 58.3 |
| 90 | 80.1 |
| 100 | 100.0 |

Correction Equations

Based on the above data, a plot of % weightM (y-axis) vs % eventsM (x-axis) was generated (FIG. 4) and a curve was fit with the following equation:

y=0.0000015901859517x⁴−0.0003883825100108x³+0.0255081044479226x²+0.7447058163287470x

Data Analysis—Test Data

The raw data from the nanopore analysis was used as input into the correction equations to calculate the starting percentage by weight from the test samples. Results are shown in Table 9.

TABLE 9
Determining % weight from % eventsM
Test Data

| | | calculated | |
|---|---|---|---|
| % weightM | % eventsM | % weightM | % error |
| 20 | 15.9 | 16.8 | 3.2 |
| 40 | 31.2 | 37.7 | 2.3 |
| 60 | 45.7 | 57.1 | 2.9 |
| 80 | 65.4 | 78.3 | 1.7 |

The calculated % weightM was highly accurate with error rates within the range of fluorescence detection.

Fractional Abundance Analysis

RR1 percentage was determined using a fractional abundance analysis as provided in PCT Publication No. WO 2018/081178, “Fractional Abundance of Polynucleotide Sequences in a Sample,” published May 3, 2018, incorporated by reference.

The SVM with Gaussian Kernel was used to model the controls and the parameters for the modeling are log 10 (duration), median amplitude and max amplitude. The results provided in Table 10 are the median of 4 separate nanopore estimates for the RR1 percentage.

TABLE 10
RR1 percentage determined by fractional abundance

| Sample | Median nanopore estimate |
|---|---|
| 0% RR1 | 1.72% |
| 60% RR1 | 56.52% |
| 70% RR1 | 65.39% |
| 80% RR1 | 78.79% |
| 90% RR1 | 85.39% |
| 100% RR1 | 100.00% |

Although the particular embodiments have been described in some detail by way of illustration and example for purposes of clarity of understanding, it is readily apparent in light of the teachings of this invention that certain changes and modifications may be made thereto without departing from the spirit or scope of the appended claims.

Accordingly, the preceding merely illustrates the principles of the invention. Various arrangements may be devised which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### Example 3: Fast and Accurate Quantification of Insertion-Site Specific Transgene Levels from Raw Seed Samples Using Solid-State Nanopore Technology

The method as described herein is a PCR-based method that is simple to design, starts from whole seeds, and can be run to end-point in less than 5 minutes. Subsequent relative quantification (trait vs. non-trait) using capillary electrophoresis performed in 5% increments across the 0-100% range showed a mean absolute error of 1.9% (s.d.=1.1%). It was showed that the PCR assay can be coupled to non-optical solid-state nanopore sensors to give seed-to-trait quantification results with a mean absolute error of 2.3% (s.d.=1.6%). In concert, the fast PCR and nanopore sensing stages demonstrated here can be fully integrated to produce seed-to-trait quantification results in less than 10 minutes, with high accuracy across the full dynamic range.

An assay method where the two amplified DNA molecules have an identical sequence on one of their ends but not the other was developed. This arrangement could be found at the genomic location of a transgene insertion, when compared to its associated wildtype variant. In a sample containing both the transgene-inserted and the wildtype templates, an end-point PCR is then performed to generate both amplicons in the same sample using three primers, one of which is common to both amplifications. Because the common primer is consumed at a higher rate than either of the other primers, it becomes a limiting reagent that therefore holds both products at close to the same amplification rate. At endpoint, the ratio of the two products is reproducibly correlated to the ratio of the starting template. Since both amplifications occur in the same tube, any inhibition due to crude DNA extractions affects both reactions at the same level, and thus has a minimal effect on the ratio of the products.

By designing primers to give different sized PCR amplicons, the end-point ratio can be determined by any method able to separate and quantify them. The most common laboratory methods for this are gel electrophoresis or capillary electrophoresis, with quantification by using a fluorescent intercalating dye or UV absorbance. Another method is to not separate the PCR products at all, and simply quantify their relative amounts by recording the change in electrical signal when individual DNA molecules translocate through a solid-state nanopore sensor [9].

Briefly, a solid-state nanopore is a nanoscale hole formed in a thin solid-state membrane that separates two aqueous volumes [10,11]. An amplifier applies a voltage across the membrane while measuring the ionic current through the open pore. When a single charged molecule such as a double-stranded DNA is captured and driven through the pore by electrophoresis, the measured current shifts, and the shift depth and duration properties are used to characterize each single-molecule “event.” After recording 100-1000 events in a few minutes, the event distributions are analyzed to characterize the corresponding molecules present [12]. Nanopore sensing thus offers a simple and high-throughput electrical read-out, with an instrument that can have a small footprint at low cost [9]. Previous studies have shown that nanopores can discriminate DNA by length, since longer DNA produce longer duration events [11,13]. For example, length-based discrimination with Bayesian classification has been used for molecular “fingerprinting” in a diagnostic application [14]. A nanopore-based method as shown herein was developed for relative quantification of two DNA populations [15], which is applied here using length-based discrimination but is compatible with any other nanopore-based scheme for DNA discrimination [16,17].

To demonstrate a use case, it was chosen to validate the method by quantification of the relative weight of soybeans that comprise the GTS40-3-2 event, which confers resistance to glyphosate [1], from a 35-gram mixture of seeds. To show the simplicity of assay design and robustness of the method, three different 3-primer assays were designed, and demonstrate insertion-site specific quantification. It was also demonstrated that the method works with crude samples, and that the PCR can be performed in under five minutes. Lastly, it was shown that the post-PCR ratio can be accurately and efficiently quantified using solid-state nanopores.

Three-Primer Assay Design and Quantification

The method was presented as shown in the following sections, and and alternative DNA extraction and PCR protocols were compatible with the method. As a proof-of-concept example, soybeans that comprise the GTS40-3-2 event (Trait Seeds) and conventional soybeans (Non-Trait Seeds) were used to make mixtures for relative quantification. These mixtures were defined by the amount of Trait Seed material in the mix (% Trait), with 0% Trait having only Non-Trait Seed and 100% Trait having only Trait Seed.

1. Obtain DNA Sequences

Genomic DNA sequence was first obtained for one of the junctions where the transgene of interest was inserted into the genome (the Trait DNA). About 400 base pairs on either side of the junction are needed. The same length of corresponding genomic sequence from the non-transgenic organism found in the mixture was also needed (the Non-Trait DNA). Half of the two sequences were identical, or nearly identical (the Common DNA). The procedure used to obtain the Trait DNA and Non-Trait DNA are found in the Genomic DNA Sequences Protocol.

2. Design the Three Primers

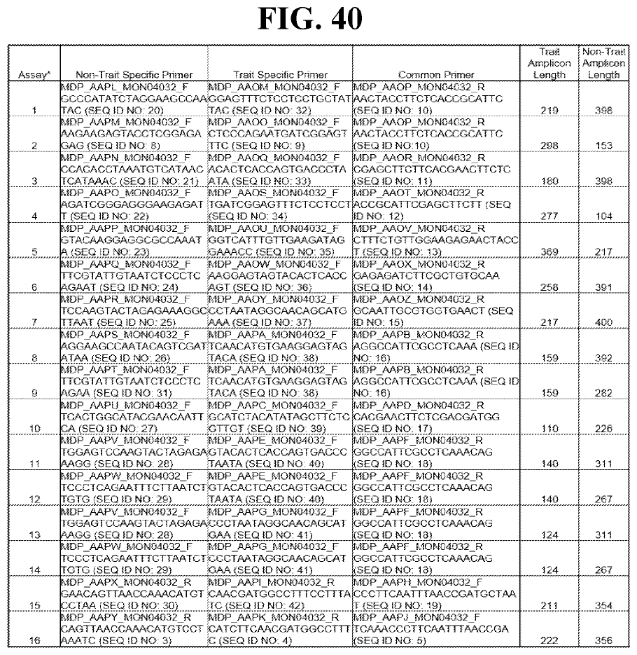

Using the PCR Primer Design Protocol with the Trait DNA, two oligonucleotide PCR primers were next designed that generated a PCR amplicon 80-400 base pairs in length (the Trait PCR) that crossed the junction in the Trait DNA. There was one primer that binds within the transgene (the Trait Primer), and a second primer that binds to the Common DNA (the Common Primer). Using the PCR Primer Design Protocol with the Non-Trait DNA, the Common Primer was used to design another PCR primer (the Non-Trait Primer) that crosses the site that was disrupted when the transgene was inserted. The amplicon generated by these two primers (the Non-Trait PCR) had a length that was sufficiently different from the Trait PCR to facilitate relative quantification of Trait vs. Non-Trait amplicons following end-point PCR. Nominally, the difference in length is at least 100 bp for facile quantification using either capillary electrophoresis or nanopore measurement. All three primers (Trait, Non-Trait, Common) together made an assay. To demonstrate diversity of primer design, sixteen different assays were made shown in FIG. 40 for the model Trait vs. Non-Trait system, three of which (assays 2, 14 and 16) were selected to showcase the full method presented here.

3. Produce Reference DNA Templates

Seed mixtures: Reference DNA templates of 0% Trait and 100% Trait seeds were produced, as well as one from a 50% Trait mixture of seeds. In this example, the Quick DNA Extraction Protocol was used to make crude extracts from whole soybeans in less than one minute. The resulting 0%, 50% and 100% extracts from seeds were denoted as “% Trait-Extract” in figures and tables.

Extract mixtures: To produce accurate mixtures that combined the 0% and 100% extracts, the extracts were normalized to the same A260 absorbance. The 0% Trait and 100% Trait extracts were then mixed by volume to make a total set of 19 additional extracts, from 5% Trait to 95% Trait, in 5% Trait increments. These extracts were denoted as “% Trait-Extract-Mix” in figures and tables.

4. Test the Assay for Specificity

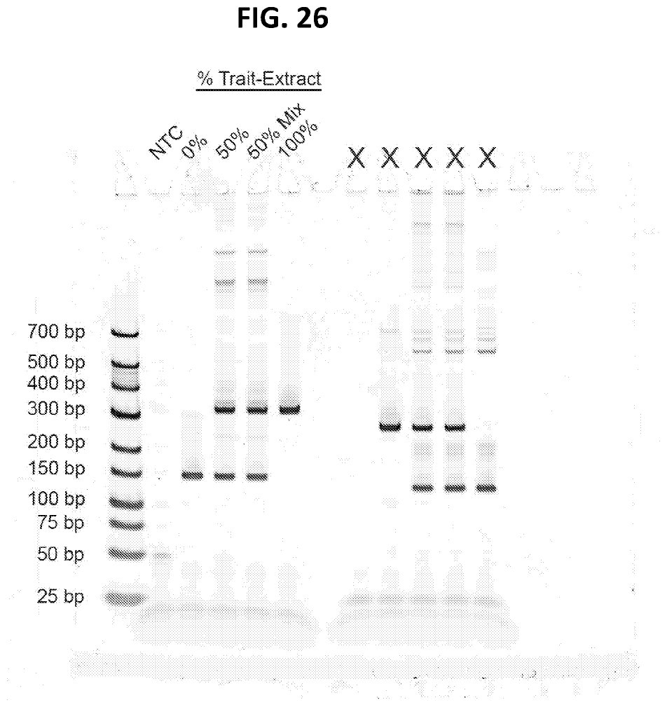

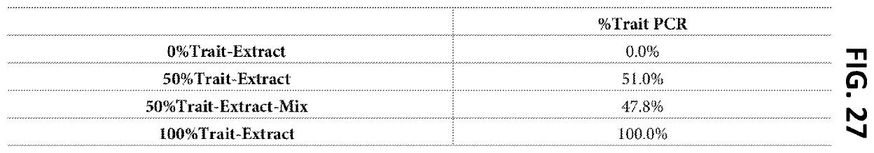

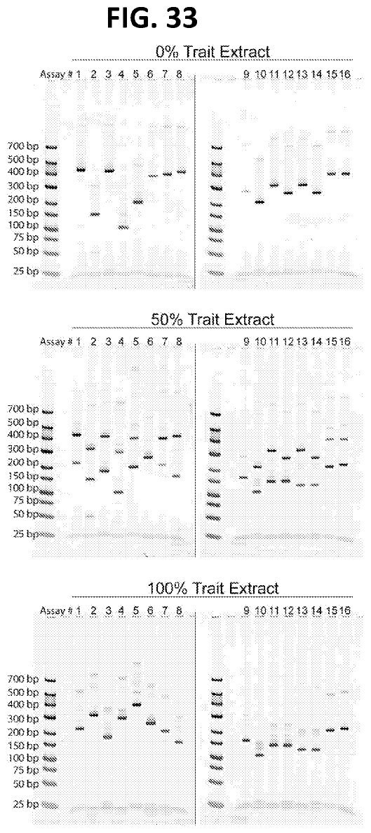

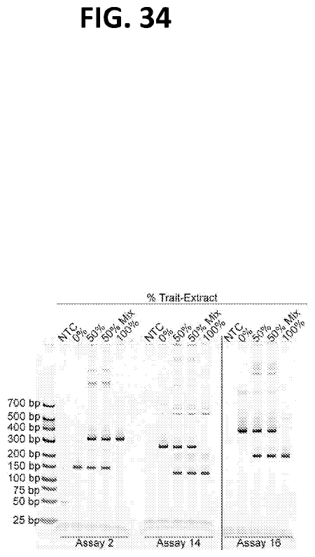

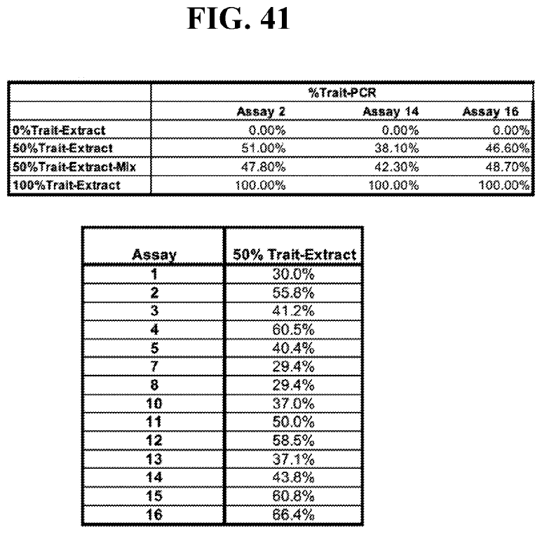

Assays were checked for specificity with the 0%, 50%, and 100% Trait-Extracts, as well as the 50% Trait-Extract-Mix. Using PCR Protocol A, all sixteen assays were tested for specificity (FIG. 33). Successful assays had single PCR amplicons for 0% Trait PCR and 100% Trait PCR, while both amplicons (Trait PCR and Non-Trait PCR) were at similar levels for the 50% Trait. The PCRs could be qualitatively visualized using the Gel Electrophoresis Protocol, as shown for assay 2 in FIG. 26, and shown also for assays 14 and 16 in FIG. 34. When visualized on a gel, the PCRs containing both templates often have bands higher up on the gel, which is likely the result of hetero-duplex formation but does not have a negative impact on the results. The PCR shown in FIG. 26 was next quantitated using the Capillary Electrophoresis Protocol. The quantification was reported as “% Trait PCR”, which is the percentage of Trait PCR (in ng) to total PCR (Trait PCR and Non-Trait PCR in ng). The % Trait PCR of the 50% Trait-Extract and 50% Trait-Extract-Mix showed close to the same value for assay 2 (FIG. 27), and also for assays 14 and 16 (FIG. 41). Quantification of 50% Trait-Extract was also tested for all sixteen assays, with assays 2 and 14 showing values within 10% of the true 50% value (FIG. 41). While Capillary Electrophoresis was used to quantitate the reactions in these examples, any method that can quantify the relative amount of the two amplicons may be used.

5. Generate a Reference Data Set

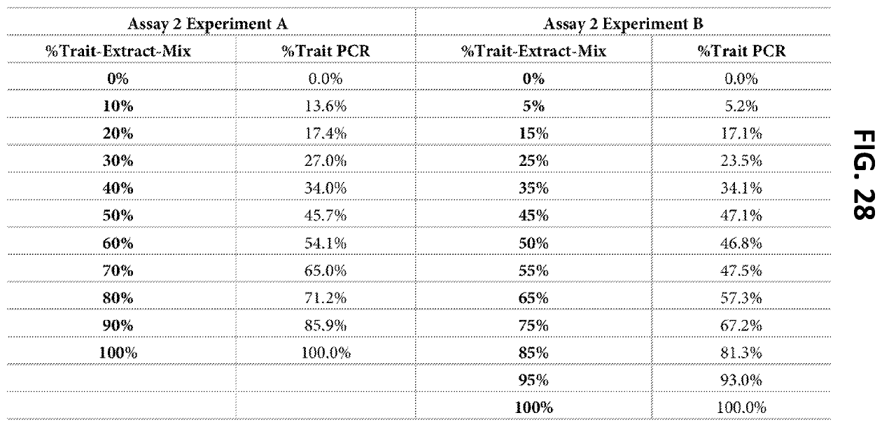

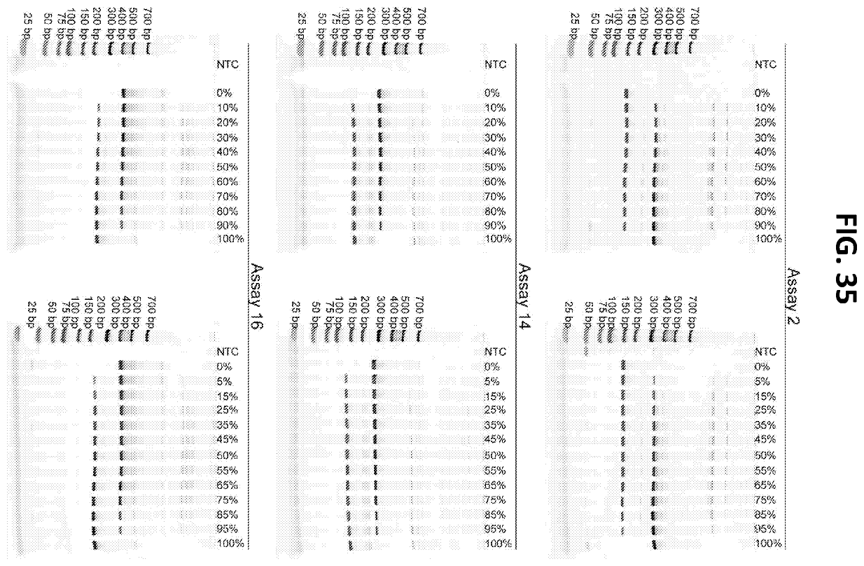

A reference data set was next created and used to make a Calibration Equation. The reference data was generated from any amount of test PCRs. The minimum number of test PCRs is a single reaction with the 50% Trait-Extract-Mix. Twenty-one % Trait-Extract-Mix reactions (0-to-100%, in 5% increments) were used, and using PCR Protocol A with assay 2. These PCRs were performed in two sets (Experiment A and Experiment B), but as long as the same protocol was used, they could be performed all together, or divided into smaller subsets. The PCRs were qualitatively analyzed using the Gel Electrophoresis Protocol (FIG. 35), and quantitatively analyzed using the Capillary Electrophoresis Protocol to yield a set of % Trait PCR values (FIG. 28).

6. Generate a Calibration Equation

The % Trait PCR values were next plotted vs the % Trait-Extract-Mix values.

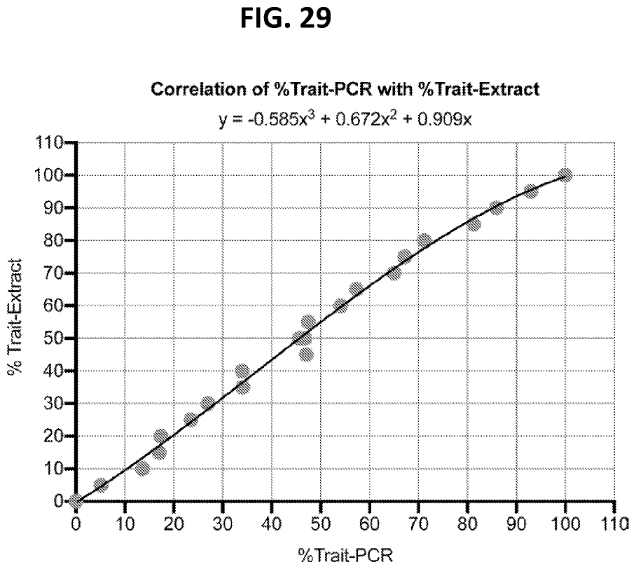

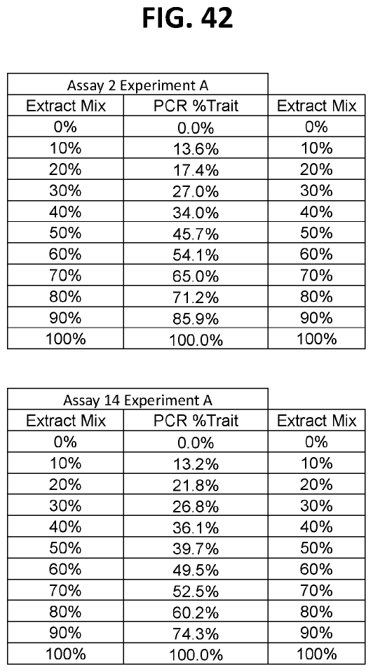

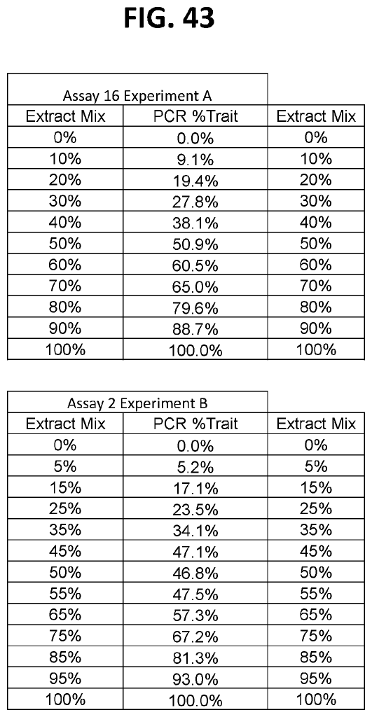

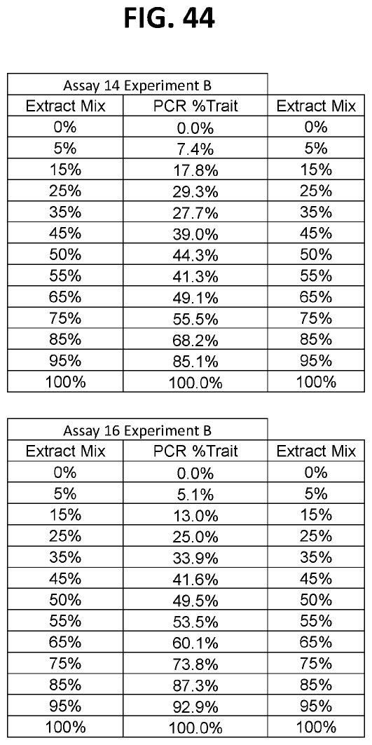

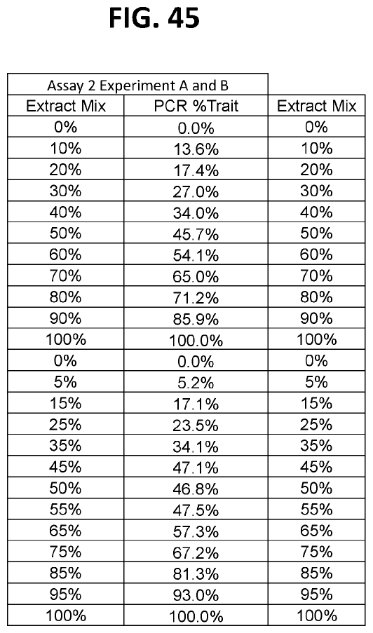

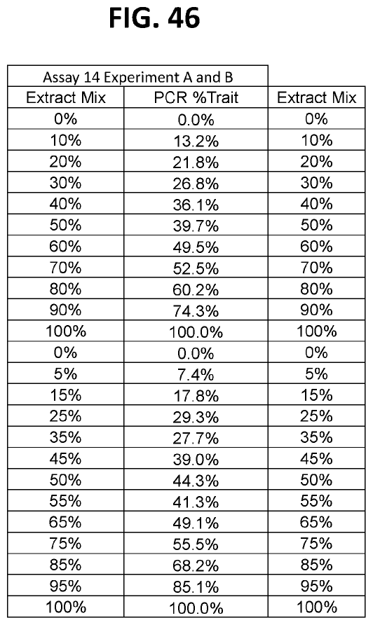

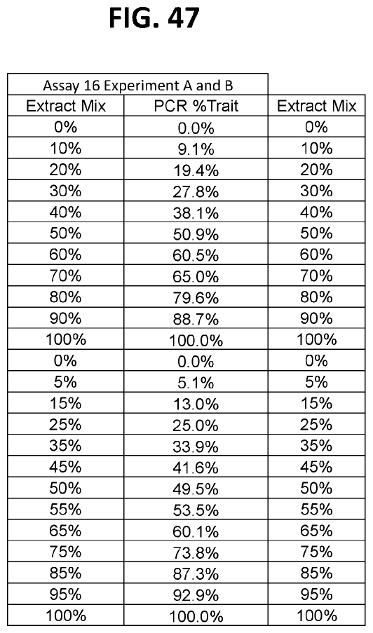

Microsoft Excel Software was used to plot the % Trait PCR values on the X-axis, and the input % Trait-Extract-Mix value on the Y-axis. The software was then used to perform regression analysis to generate a 3^{rd} degree Calibration Equation using all 21 data points combining Experiments A and B (FIG. 29). To demonstrate that this can be done with fewer points, a fit was also made using only the 50% Trait PCR (and anchored at (0,0) and (100,100)) to generate a 2^{nd} degree Calibration Equation. For the assay 2 reference data, for example, the equation produced using only the 50% Trait PCR was: y=−0.151x²+1.151x, where x is % Trait PCR and y is % Trait. The 3^{rd} degree and 2^{nd} degree Calibration Equations calculated for assays 2, 14 and 16 using Experiment A and B data were reported in FIGS. 42-47. Note that by having % Trait PCR values on the X-axis, test data produced from raw seed mixtures can be analyzed (as described in the next section) to produce a % Trait PCR value as the X value, and the equation could be directly applied to produce a % Trait-Extract estimate as the Y value output.

7. Produce Test DNA Templates

Test DNA templates of mixed Trait and Non-Trait organisms were produced next. For this example, 21 mixes of whole soybeans were weighed out, and used the Quick Extraction Protocol on each to make 21 different % Trait extracts, from 0% Trait to 100% Trait in 5% Trait increments. The test extracts were not normalized to a certain A260 reading, in part to emulate the condition of testing from crude seed-mixture extracts. These test extracts are noted as “% Trait-Extract” in figures and tables.

8. Generate Test Data

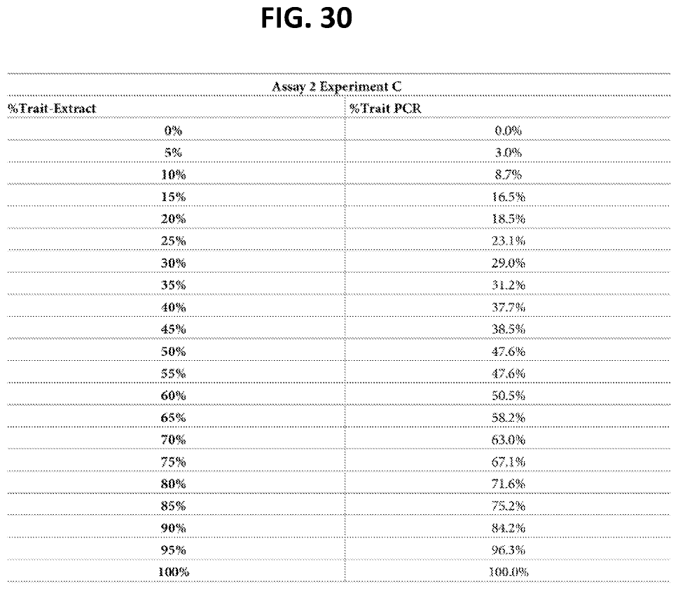

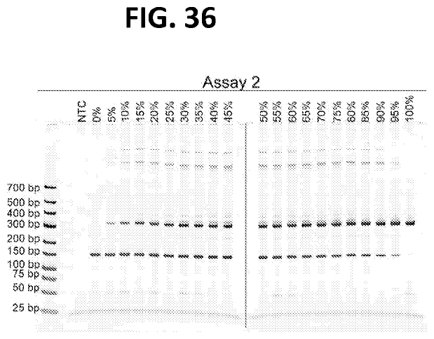

Using exactly the same protocols used to produce the % Trait PCR values in the reference data sets, test samples could be used to produce test % Trait PCR values. In this example, the 21 test DNA templates made in step 7 were used with assay 2 and PCR Protocol A to create a test set with 21 test reactions (termed “Experiment C”). The test set was qualitatively analyzed with the Gel Electrophoresis Protocol (FIG. 36), and quantitatively analyzed with the Capillary Electrophoresis Protocol to produce a set of test % Trait PCR values (FIG. 30).

9. Calculation of % Trait-Extract from % Trait PCR

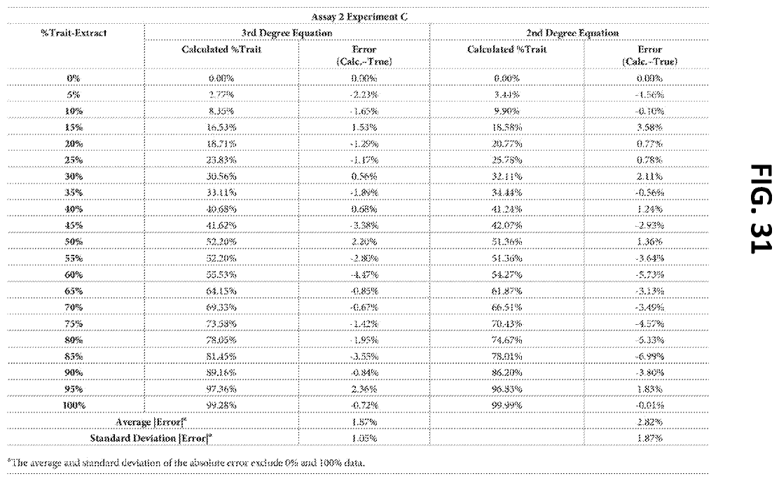

Using the calibration equations generated in step 7, the % Trait-Extract values could be estimated from the % Trait PCR values. For this example, the % Trait-Extract values were calculated from each of the 21 tests of Experiment C using the calibration equations that were derived from Experiments A and B data. When using the 3^{rd} degree equation, the average absolute error between the true % Trait-Extract and the calculated value was 1.87%, with the largest error of −4.47%. Using the 2^{nd} degree equation, the average absolute error was 2.82%, with no individual difference of more than 7% (FIG. 31). The mean and standard deviation of the absolute error values reported at the bottom of FIG. 31 excluded the 0% and 100% error values, since there corrected values had nearly zero error by design of the calibration method.

Protocols

Soybean Varieties Protocol

All assays were tested using either soybeans (Trait soybeans) containing the GTS40-3-2 event that confers glyphosate tolerance (a blend of Big Fellow, Large Lad, and Whitetail Thicket), a non-transgenic heirloom ‘Black Jet’ soybean (Non-Trait soybeans), or a defined weight/weight mixture (% Trait) of the two soybean varieties.

Genomic DNA Sequences Protocol

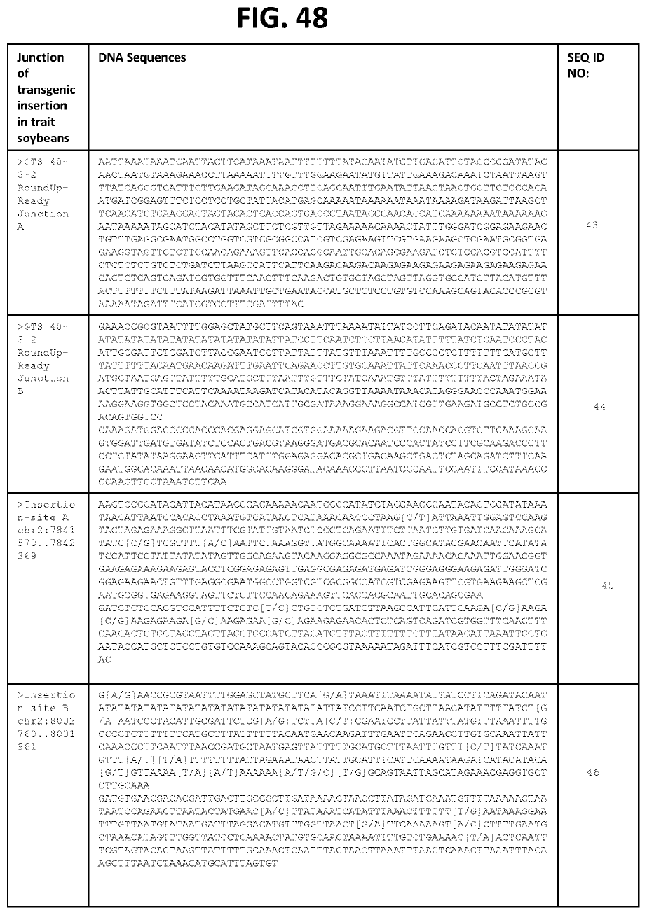

The DNA sequences (FIG. 48) at the junctions of the transgenic insertion in Trait soybeans were taken from the literature [18]. As the transgene was integrated into the genome with a complex rearrangement, the two ends (Junction A, and Junction B) were connected to two genomic locations that are not contiguous in conventional soybeans. The DNA sequences of the two corresponding insertion sites in conventional soybeans (Non-Trait) were obtained with a BLAST search of the database of the Legume Information System (www(dot)legumeinfo(dot)org/home) using the transgene junction sequences.

Primer Design Protocol

Three 3-primer sets of oligonucleotide primers (assays 2, 14 and 16 in FIG. 40) were designed using PrimerQuest software (Integrated DNA Technologies www(dot)idtdna(dot)com/PrimerQuest/) with the following parameters: Tm 59° C.-65° C., GC content 35%-65%, length 17 nt-30 nt. In order to design 3-primer sets, first 2-primer sets were designed to give Non-Trait amplification products between 75 nt and 400 nt in length, that include the DNA sequence that was disrupted when the transgene was integrated into the chromosome. Primers used to amplify these Non-Trait fragments were screened to avoid all known DNA variants using the Soybean Genome Variation Map (BIGD www(dot)bigd(dot)big(dot)ac(dot)cn/gvm/search). Each of the acceptable primers was then individually used as a starting point to design a primer that would amplify only Trait DNA, and not Non-Trait DNA. Because the transgene insertion event is not a simple insertion, both Junction A and Junction B were chosen to design assays. The amplification product lengths were chosen to make pairs of DNA amplicons (one amplified only from Non-Trait soybeans, and one amplified only from Trait soybeans) differing by at least 100 bp for facile quantification.

DNA Extraction Protocol

35 grams of soybeans (˜250 seeds) were ground with an Oster blender on the highest setting for 10 seconds, and 100 mL of 250 mM NaOH was added to the ground seeds and shaken by hand for 15 seconds. Roughly 75% of the contents in the cup were poured into an Aeropress coffee maker and filtered with a paper filter, yielding 10-15 mL of filtered extract. Using a blunt p1000 pipette tip, 1 mL of filtered extract was diluted 1:10 by adding 9 mL of water. The mixture was vortexed for 10 seconds, then another 1:10 serial dilution was performed in 5 mL final volume, for a final dilution of 1:100. The 1:100 diluted extract was then aliquoted and frozen at −20° C., diluted further, or directly used as the template for the PCR reaction.

PCR Protocol A

4μL of a 1:1000 dilution from a Quick DNA Extraction was added to 21 μL of PCR master mix (5μL 10× HF buffer (New England Biolabs), 0.5 μL Phusion Hot Start Flex DNA Polymerase (New England Biolabs), 0.5 μL 10 mM dNTPs, 0.5 μL 30 μM each of 3 oligonucleotide primers (Integrated DNA Technologies), 13.5 μL water for a final volume of 25 μL) The amplifications (in triplicate) were performed using a C1000 Touch thermocycler (Bio-Rad) [95° C. for 30 sec, followed by 35 cycles of 95° C. for 5 sec, 60° C. 10 sec, 72° C. 10 sec, followed by 72° C. for 30 sec]. The triplicate amplification reactions were then merged to a final volume of 75 μL before analysis.

PCR Protocol B

2 μL of a 1:100 dilution of each of extract was added to 10.5 μL of PCR master mix (2.5 μL 10× HF buffer (New England Biolabs), 1.5 μL Phusion Hot Start Flex DNA Polymerase (New England Biolabs), 0.25 μL 50 mM MgCl2, 0.25 μL 10 mM dNTPs, 0.75 μL of Non-trait specific and 1 μL of Trait specific and Common oligonucleotide primers at 100 μM (Integrated DNA Technologies), 3.254, water for a final volume of 12.5 μL). 12.5 μL amplifications (in triplicate) were performed using a NextGenPCR thermocycler (Molecular Biology Systems (MBS)) [98° C. for 5 sec, followed by 35 cycles of 98° C. for 1 sec, 55° C. 1 sec, 75° C. 3 sec, for a total run time of 4 min 52 sec]. The triplicate amplification reactions were then merged to a final volume of ˜37 μL.

Gel Electrophoresis Protocol

3 μL of each of the merged PCR products was analyzed using a 6% TBE PAGE gel run at 200V for 25 minutes, followed by staining with SybrGreen for 15 minutes and visualization with a Bio-Rad ChemiDoc MP.

Capillary Electrophoresis Protocol

2 μL of a PCR reaction was quantitatively analyzed using a Fragment Analyzer System (Agilent) and a dsDNA 910 reagent kit. The percentage of the total ng (Trait and Non-Trait) that was contained in the trait specific amplicon was recorded as % Trait PCR, and used for analysis.

Nanopore Protocol

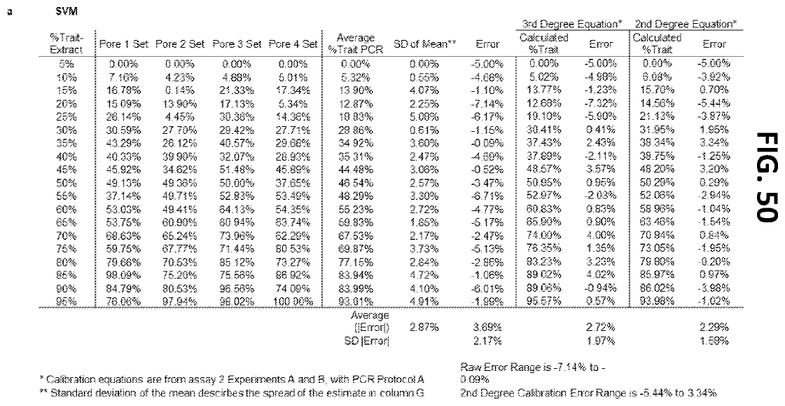

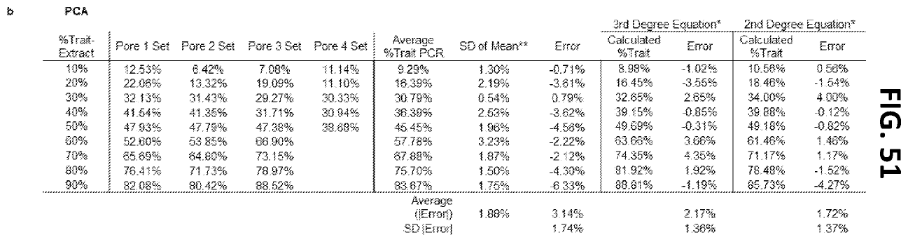

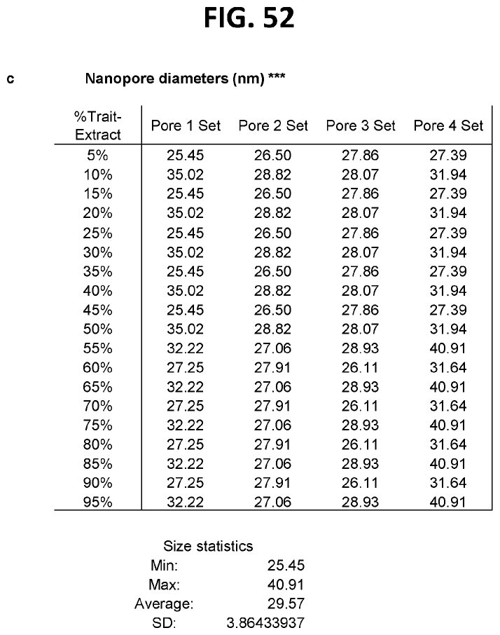

PCR reactions were diluted 1 to 50 into a nanopore recording buffer, which comprised of 4.0 M LiCl, 50 mM Tris HCl pH 8.8, 5 mM EDTA, and 10% PEG 200 v/v. Nanopore chip fabrication and the injection molded test strip used to package and fluidically seal a chip are described in “S1 Text”. For measuring a sample, approximately 10 μL of diluted sample was pipetted into the test strip and 100 mV bias was applied to the nanopore chip (trans side positive) using a prototype voltage-clamp amplifier [9]. Ionic current data was recorded using custom software at a sampling rate of 125 kHz for approximately 5 minutes, or enough time to collect ˜1000 molecular translocation events for each reagent. Each sample % Trait-Extract was recorded on 4 independent pores. Nanopore diameters ranged in size from 25-41 nm across all data sets (pore size range is discussed in “S1 Text”, and size details per nanopore device are reported in FIGS. 50-52. Control datasets, for model training and quantification correction, were collected for each pore just prior to each test data, as described in “S2 Text”. (This subject matter is related to PCT Application No. PCT/US2019/050087, unpublished).

(S1 Text):

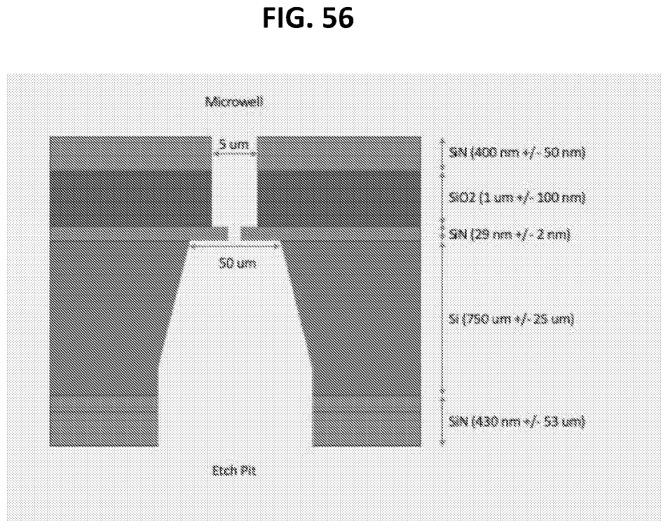

The nanopore chip: First, 30 nm of low-stress low-pressure CVD (LPCVD) SiN thin film (<200 MPa, tensile) is deposited on a 750 um Si substrates (Thermco LPCVD Nitride). The nanopores are formed in the SiN membrane by first patterning with PMMA and then exposing the 30 nm nanopore pattern using electron beam lithography (EBL) (JEOL JBX-6300 Lithography System) followed by reactive ion etching of the nanopore (RIE Oxford PlasmaPro 80). After the etch the final diameter falls within 25-35 nm. To reduce noise, an insulating layer, consisting of 1 um SiO2 layer, was deposited on the front side of the wafer using a plasma-enhanced CVD (PECVD) (PlasmaTherm Shuttlecock PECVD System) process followed by a 1000 C anneal for one hour (Thermco Oxidation Furnace). An additional 400 nm SiN etch mask layer was deposited via LPCVD (Thermco LPCVD Nitride) on the substrate following the anneal. The etch pit was opened from the backside by photolithography followed by reactive ion etching of the SiN etch mask layer (RIE Oxford PlasmaPro 80). A second photolithography step was performed on the front side of the wafer to define the SiO₂ micro-well pattern. Subsequently, reactive ion etching (RIE Oxford PlasmaPro 80) was used to partially open the SiN mask and SiO₂ layer with target etch depth of 0.8 um. The SiN membrane was then fully released by removing the remaining oxide and Si material from both sides of the wafer using a KOH wet etch. First, while protecting the frontside of the wafer, a KOH wet etch removed the Si substrate from the etch-pit side. Second, while protecting the backside, another KOH wet etch removed the the remaining oxide material from the frontside, fully releasing the SiN membrane and nanopore. A schematic of the nanopore chip is shown in FIG. 56.

Injection molded test strip—assembly and use: The test strip is shown schematically in exploded and assembled views in FIG. 2. The test strip top and base are injection molded in clear Polycarbonate (Makrolon 2407-5500115). The test strip chip and channel seal is injection molded in elastomer (211-45 Santoprene). The electrodes are screen printed Ag/AgCl ink (Creative Materials 113-09S) on 5 mil PET sheeting with an anti-abrasive Carbon coating on the connecting end (Creative Materials 124-50T). Prior to assembly, polycarbonate and elastomer parts are cleaned with 99.5% IPA in an ultrasonic cleaner (Digital Pro+) for three (3) minutes, flushed with deionized (DI) water, and dried in a food dehydrator (Excalibur 2900ECB) at ambient temperature. The nanopore chip and channels are sealed by compression of the central elastomer seal. Two screws and nuts are tightened to 6 ozf. in. with a calibrated torque screwdriver (Mountz TLF-IFR) for even compression of the seal to 25% to ensure leak-free sealing such that electrical conductivity between the channels can occur only through the nanopore. Each channel is approximately 8 microliters in volume. Prior to reagent testing, assembled test strips were prepared as follows. Test strips were filled with 10 uL of buffer in both the cis and trans channels, and the strips were loaded into the custom voltage-clamped amplifier [1]. Square voltage pulses 0.2 s in duration and ranging from ±2V to ±12V in magnitude were used to incentivize nanopore wetting. Following wetting, nanopore fitness was assessed by the symmetry of conductance over a voltage sweep from −0.3V to 0.3V, and by the root-mean-square of the current (IRMS) at 0.1 V. Pores with asymmetry <10% and IRMS <30 pA were used for reagent testing. Nanopore sizes estimated from the current, following the method detailed in [2], ranged from 25-35 nm at the start of reagent testing. Nanopores grew up to 40 nm in diameter in some cases during the process of reagent testing, for a total diameter range of 25-40 nm across all data provided in the paper.

### REFERENCES FROM S1 TEXT

1. Morin T J, McKenna W L, Shropshire T D, Wride D A, Deschamps J D, Liu X, et al. A handheld platform for target protein detection and quantification using disposable nanopore strips. Sci Rep. Nature Publishing Group; 2018 Oct. 4; 8(1):14834.
2. Morin T J, Shropshire T, Liu X, Briggs K, Huynh C, Tabard-Cossa V, et al. Nanopore-based target sequence detection. Wanunu M, editor. PLoS ONE. 2016 May 5; 11(5):e0154426-21.

Results

In one example, the workflow was simplified without using a reference-data-derived calibration, in which case the % Trait PCR values provided direct estimates for the % Trait values. However, this was implicitly equivalent to assuming a calibration equation equal to a straight line through (0,0) with slope 1, which generally produced higher errors. For the data in FIG. 30, for example, the mean absolute error (excluding 0% and 100%) was 4.64% (s.d. 3.07%), which was clearly inferior to the results using reference data to derive the calibration equations.

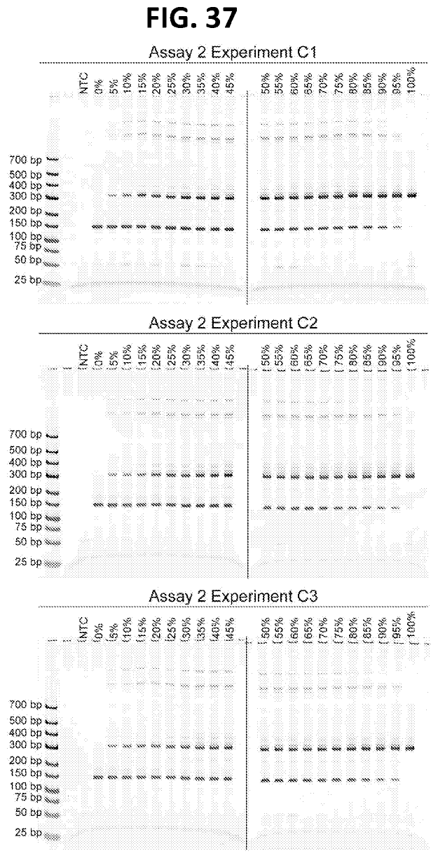

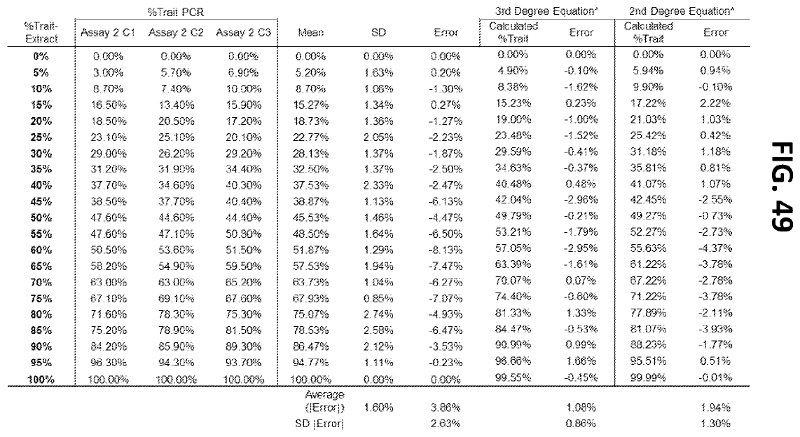

Across the entire dynamic range, the absolute error with 3^{rd} degree calibration equation had the mean value 1.87% and standard deviation 1.05% across 19 error values, which corresponded to 0.24% standard error of and a 95% confidence interval of 1.39% to 2.34% (mean±0.47%). To test variability, Experiment C data was repeated two more times (Experiments C1-C3, FIG. 37), and all three sets of results produced consistent results. Moreover, by averaging across the three sets, the error was further reduced (FIG. 49). Specifically, the triplicate-average of the mean absolute error (excluding 0% and 100%) is 3.86% (s.d. 2.63%) without calibration, and 1.08% (s.d. 0.86%) and 1.94% (s.d. 1.30%) with 3^{rd} degree and 2^{nd} degree calibration, respectively. The triplicate-averages had a mean standard deviation of 1.6%.

Using Solid-State Nanopores to Quantify % Trait

To show that the method outlined above is not limited to a particular method of quantification, the use of nanopore technology was also demonstrated for measuring and calculating the % Trait using the Experiment C samples. The nanopore-based trait quantification method is described in detail in [15] and “S2 Text”, with relevant portions described here.

Using the Nanopore Protocol, sets of four independent nanopores were used to measure and quantitate each % Trait-Extract from the Experiment C samples. Prior to running % Trait-Extract samples, three controls are sequentially run on each nanopore: 0% Trait-Extract, 50% Trait-Extract, and 100% Trait-Extract. The controls are used to build a support vector machine (SVM)-based model for assignment of trait events vs. non-trait events, and also to compensate for a difference in the nanopore capture frequency of the two different length amplicons 2 (Trait 298 bp, Non-trait 153 bp). While the % Trait-Extract values were used as internal controls for nanopore quantification, the resulting % Trait PCR estimates can be subsequently calibrated using the calibration equations derived from the % Trait-Extract-Mix reference data (Experiments A and B). Notably, those calibrations were derived using capillary electrophoresis results, and a calibration based on nanopore-analyzed reference data could further improve accuracy, though this was not explored. As with the capillary electrophoresis results for Experiment C reagents, the quadruplet nanopore measurements were generated for 0% to 100% in 5%-increments (21 values).

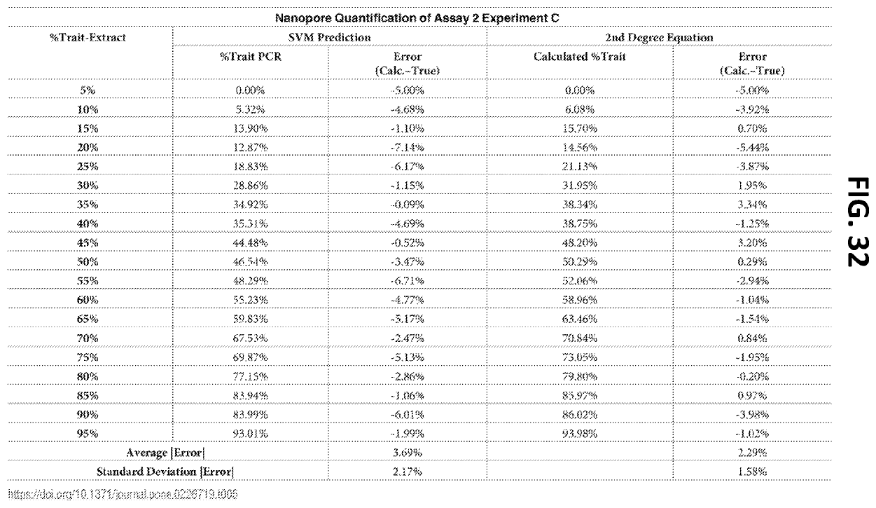

The results of applying the SVM method to quadruplet nanopore reads are shown in FIG. 32. Each of the reported % Trait PCR values are the average of the four values generated with four separate nanopores (FIGS. 50-52). As with the capillary electrophoresis results for Experiment C, the % Trait PCR estimates consistently under predicted the % Trait-Extract value (FIG. 30), and quantification improved for both methods (CE, nanopore) by using calibration (FIGS. 31-32). Using the SVM method, the largest difference between % Trait-Extract and %-Trait PCR was −7.14% and the average absolute error was 3.69% (s.d. 2.17%). The spread of each SVM prediction (defined as the standard deviation of the mean) has an average value of 2.87%. The 2^{nd} degree calibration equation derived for assay 2 using reference mixtures (Experiments A and B) was applied to the SVM data, which reduced the average absolute error to 2.29% (s.d. 1.58%) and lowered the maximum deviation to −5%.

To shorten the total assay time, the three-control workflow required was reduced by the SVM method, and developed a single-control method based on principle component analysis (PCA).

(S2 Text):

The PCA method only requires a 50% Trait-Extract to be run prior to the % Trait-Extract to be quantified. The method was applied to a subset of the same data used with SVM analysis, by removing the 0% Trait-Extract and 100% Trait-Extract data and using only the 50% Trait-Extract for correction and the “unknown” mixtures to be estimated. This was done for eleven % Trait-Extracts, from 0% to 100% in 10% increments. The PCA method produced an average absolute error of 3.14% (s.d. 1.74%), which was further improved to 1.72% (s.d. 1.37%) by applying the 2^{nd} degree calibration equation nanopore (FIGS. 50-52). The maximum deviation was −6.33% before calibration, and −4.27% after calibration.

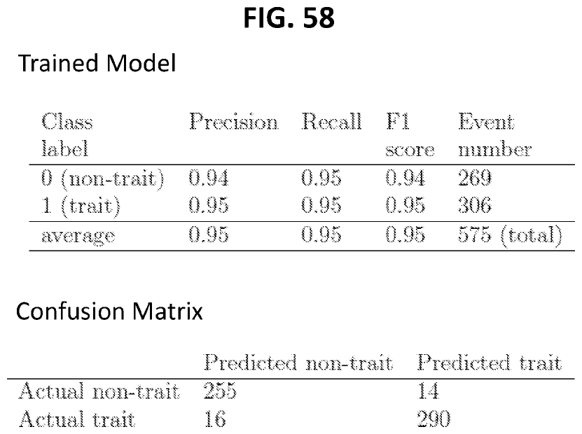

Trait vs. Non-Trait Relative Quantication from Nanopore Data: The PCR products were measured using solid-state nanopore sensors, building on our previous development of that technology [1, 2]. Briefly, the translocation of DNA fragments through a nanopore generates a measurable current signature, and trait and non-trait fragments can be discriminated by differences in their respective event signatures. Support vector machines (SVMs) are used for classifying translocation events [3]. First, two control samples (100% trait DNA with 0% non-trait DNA; and 0% trait DNA with 100% non-trait DNA) were run sequentially on a pore, each run collecting a comparable number of events, and the data were combined to train the model. Event signatures were defined as features and include: duration, median amplitude, max amplitude and area. The combined data is divided (70:30) as training: testing data. For the SVM algorithm, a hyper-parameter grid search on the training datasets was used to find the optimal model (optimizing ROC AUC score with 5-fold cross-validation). After the grid search finds the optimal model using the training data, the test data was classified using that model and scored, as shown in the table in FIG. 58.

False negative and false positive (FN/FP) rates and model accuracy are derived from this matrix. Next, a control mixture with a known ratio X:Y of trait: non-trait molecules (nominally, 1:1) was run on the same pore, and is used to correct for the difference in capture frequency between the trait and non-trait molecules. The equations and mathematical method for using the FN/FP and control mixture were detailed in [4], and summarized here. The estimated fraction of trait molecules in the unknown mixture is denoted Fmix and is given by the equation:

$${F}_{mix} = \frac{ρ α}{ρ α + 1} , where ρ = ( \frac{{Q}_{mix} - {Q}_{0}}{{Q}_{1} - {Q}_{mix}} ) , \\ α = ( \frac{{Q}_{1} - {Q}_{X : Y}}{{Q}_{X : Y} - {Q}_{0}} ) \times \frac{X}{Y} . \qquad ( 1 )$$

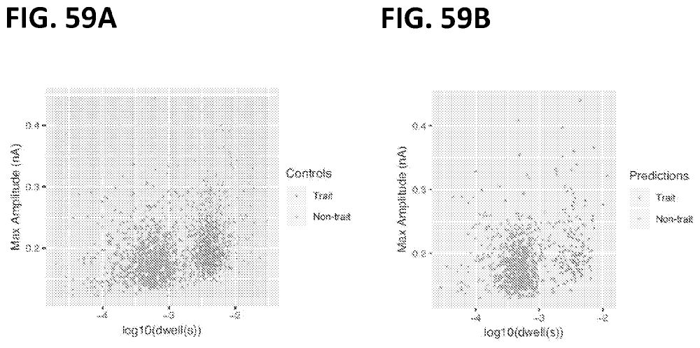

The four variables Q₁;Q₀; Q_{X:Y}; Qₘᵢₓ are the fraction of model-identified trait events for each of the four reagents run on the pore: the first three control regent sets (100% trait, 100% non-trait, X:Y control mixture), and the unknown mixture. FIGS. 59A-59B shows representative nanopore event populations from the first two control regent sets (100% trait, 100% non-trait) overlaid along with the model-identified boundary between trait and non-trait events (FIG. 59A), and the results of the model-identified event binning applied to an \unknown” mixture (FIG. 59B) that is 30% trait.

The SVM prediction after applying equation (1) to the data in FIG. 59B is 27.7%, compared to the known value of 30% trait. The FN/FP were 6.0% and 3.9% from a total of 1008 and 907 events recorded, respectively. The control mixture produced Q_{1:1}=0:3192 fraction of model-identified trait events out of 943 total events, while the unknown mixture produced Qₘᵢₓ=0:1717 fraction of model-identified trait events out of 967 total events.

The results presented in the main text and in S6 Table combined the SVM predictions of four independent nanopore results. Each nanopore runs three controls and one or more (but less than five) mixtures that were treated as unknowns. The combined predictions are the mean of the four predictions generated for a common % Trait value across four independent nanopores.

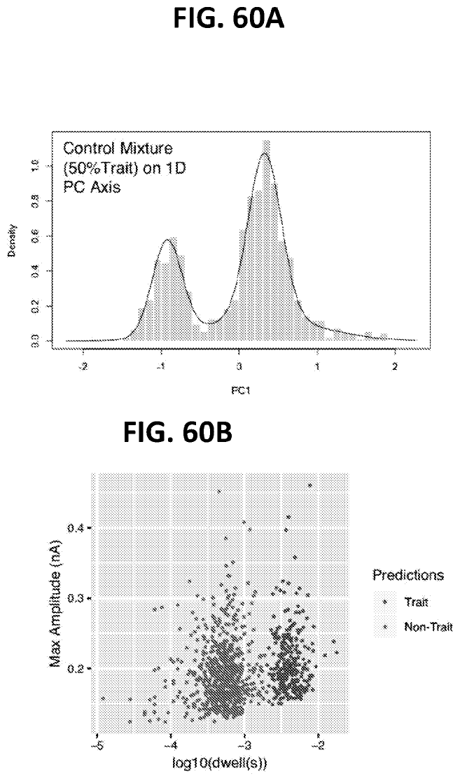

Since the SVM method requires three controls, a single-control based method was developed and applied to reduce the number of controls required for prediction. The alternative method uses principle component analysis (PCA) [5], and requires only the control mixture. The method found the best linear combination of event parameters that maximally divided the control mixture into two subsets on a single axis (FIGS. 60A-60B). The dividing line between the subsets is then used to identify trait vs. non-trait events from the unknown mixture (no FN/FP correction is performed), and a control mixture ratio is still used as a correction. This is equivalent to applying equation (1) and setting Q₁=1 and Q₀=0.

The PCA prediction for the same data shown in FIG. 59B is 30.3%. The raw value for the control mixture was Q_{1:1}=0:2916 model-identified trait events out of 943 total events, while the raw unknown mixture produced Qₘᵢₓ=0:1520 fraction of model-identified trait events out of 967 total events. The results of applying the PCA method are provided in the tables in FIGS. 50-52.

### REFERENCES FROM S2 TEXT

[1] Trevor J Morin, William L McKenna, Tyler D Shropshire, Dustin A Wride, Joshua D Deschamps, Xu Liu, Reto Stamm, Hongyun Wang, and William B Dunbar. A handheld platform for target protein detection and quantification using disposable nanopore strips. Scientific Reports, 8(1):14834, October 2018.
[2] Trevor J Morin, Tyler Shropshire, Xu Liu, Kyle Briggs, Cindy Huynh, Vincent Tabard-Cossa, Hongyun Wang, and William B Dunbar. Nanopore-based target sequence detection. PloS ONE, 11(5):e0154426{21, May 2016.
[3] N Cristianini and J Shawe-Taylor. An introduction to support vector machines and other kernel-based learning methods. Cambridge University Press, 2000.
[4] Yanan Zhao, William Mckenna, and William B Dunbar. Fractional abundance of polynucleotide sequences in a sample, International patent no. WO2018081178A1, May 2018.
[5] Ian Jolliffe. Principal Component Analysis. Springer, Berlin, Heidelberg, 2011.

Reducing PCR Time

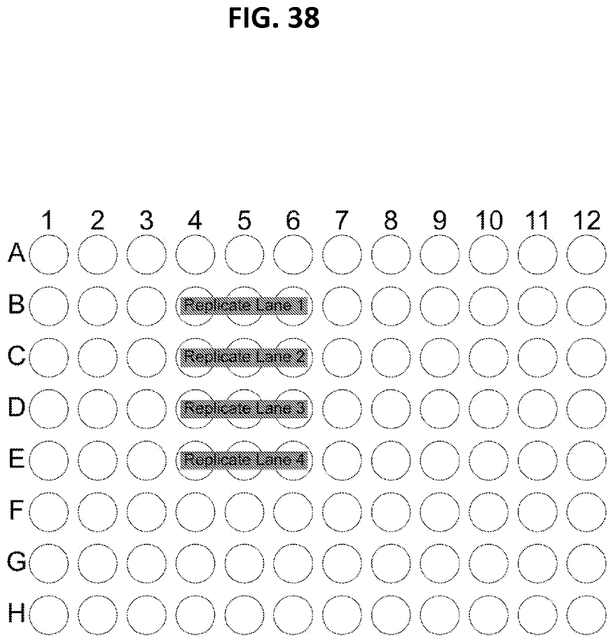

To demonstrate that the method is compatible with rapid PCR, reaction conditions were adjusted to complete 35 cycles (i.e., end point) in less than five minutes using assay 14 and a fast PCR device (MBS, PCR Protocol B). During testing of the device, different positions on the 96-well plate were observed to generate different % Trait PCR values. To compensate for this, each PCR pooled the results from three adjacent reaction wells (PCR Protocol B). To test reproducibility, four replicates lanes were run in parallel (FIG. 38). Each replicate lane was used to generate % Trait PCR values for a total of eleven % Trait-Extracts, from 0% to 100% in 10% increments. As before, extracts were made from seed mixtures with the Quick Extraction Protocol.

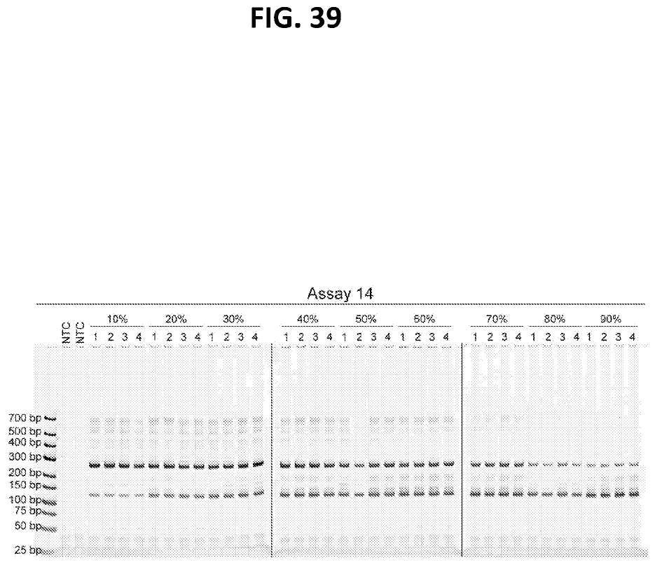

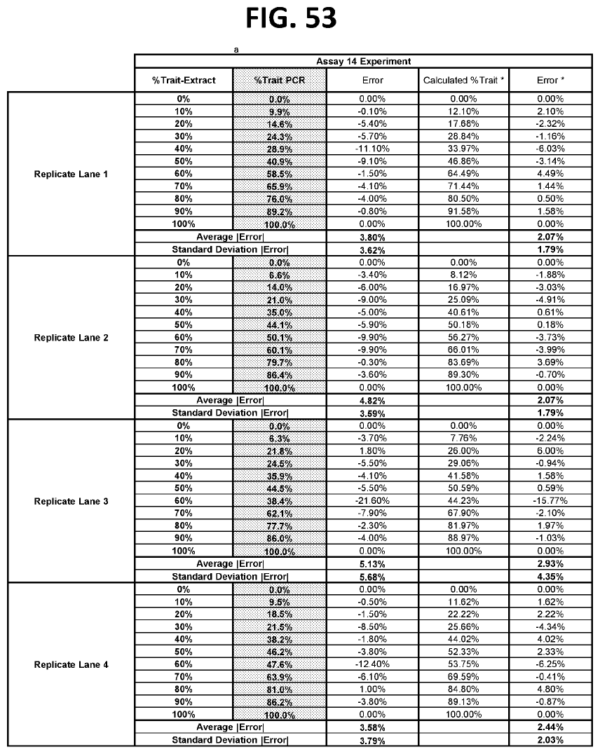

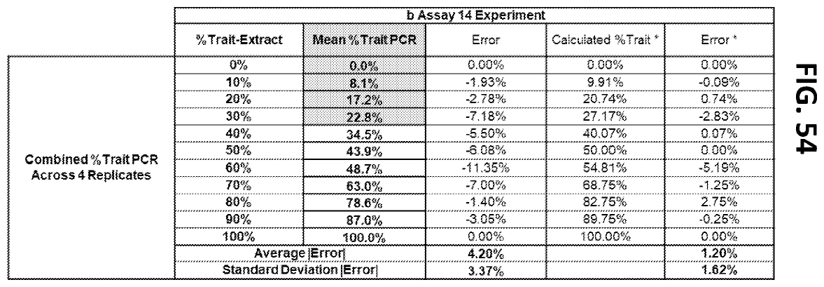

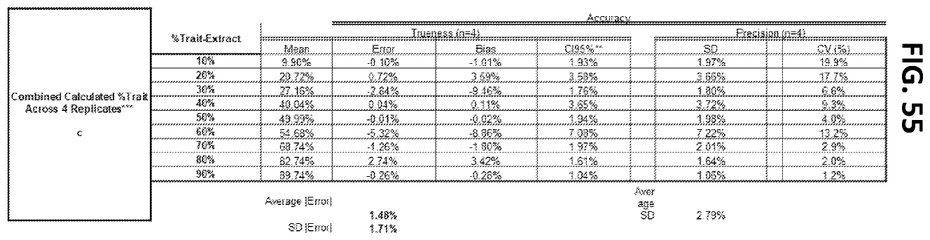

Using the same protocols used to produce the % Trait PCR values in the reference and test data sets, fast PCR samples can be used to produce test % Trait PCR values. The fast PCR samples were qualitatively analyzed with the Gel Electrophoresis Protocol (FIG. 39), and quantitatively analyzed with the Capillary Electrophoresis Protocol to produce a set of test % Trait PCR values (FIGS. 53-55). Reference material was not run on the fast PCR device. To provide a Calibration Equation correction option, the average 50% Trait-Extract PCR value across the 4 replicate lanes (43.9%) was used as a proxy for the 50% Trait-Extract-Mix value, resulting in the 2^{nd} degree equation: y=−0.24664x²+1.24664x. Similarity of the 50% Trait-Extract and 50% Trait-Extract-Mix value for assay 14 (FIG. 41) suggests that the calibration equation should be similar to what would be produced with an averaged 50% Trait-Extract-Mix value generated with the fast PCR device.

When using the 2^{nd} degree equation, the average absolute error between the true % Trait-Extract and the calculated value varied from 2.07%-2.95% across the 4 replicate lanes, with lane 3 showing the largest error of −15.77% at the single value of 60% Trait (suggesting it was an outlier). Combining the calculated % Trait values across the 4 replicate lanes resulted in an average absolute mean error of 1.48% (s.d. 1.71%) and average standard deviation of 2.79% (FIGS. 53-55). Thus, with greater redundancy in the workflow, averaging can reduce errors. The largest error was the combined estimate for 60% at −5.32%, again primarily being weighted by the outlier of lane 3. An outlier removal strategy could remedy this issue.

As before, it was also observed that without calibration the errors are higher: the average absolute error across the 4 replicate lanes is 4.2% (s.d. 3.37%). Across the replicates, bias and coefficient of variation (CV) was also reported in FIGS. 53-55 using a format consistent with W, but reiterate that scaling by the mean results in relative error comparisons while the statistics of the absolute error was focused on.

The protocol presented in example 3 provides a method for calculating the relative amount of a transgene, at a unique insertion site, from a weighed sample of seeds. As long as the reference experiments were performed in the same manner as the test experiments, it is also highly accurate across the entire dynamic range (5-100% shown here).

Although PCR amplified reference samples was used to generate the calibration equations, this need not be the case once an assay has been defined. It was demonstrated that the same equations can be used to analyze experiments performed at different times and from different seed extractions. It was also shown that as little as one reference data point (namely, the reference 50% Trait reagent) could be used to make a calibration equation that provides accurate results, and additional reference data points were shown to improve accuracy by improving the calibration equation.

True errors could also result from a dilution of the extract causing a sampling error. With this in mind, the dilution limit of the quick extracts was tested, and found that they still give quantitative results when diluted 200,000 times. While the maximum sampling error for a sample of 2,000 is +/−2.19% (at a 95% confidence level), each of the measurements were performed on a physical mixture of three independent PCRs, which could bring the potential sampling error down closer to +/−1%.

The three 3-primer assays presented here for the GTS40-3-2 event all worked with a common PCR protocol. By contrast, methods that use isothermal amplification require more complicated primer sets that loop or contain restriction sites, and optimization is not as straightforward.

Two different technologies were used to measure the ratio of the two PCR amplicons, capillary electrophoresis and solid-state nanopores, with comparable results. In practice, a number of other methods could be used to measure the ratio, such as gel electrophoresis, sequence specific fluorescent probes, or separation of the molecules with affinity tags on the primers, followed by quantification. For technologies where the separation of molecules must be compared to a reference ladder, the optimal 50% reference sample can be synthetically made from the proper amounts of the two DNA molecules, and can be run before or after the test sample.

By combining quick extraction and fast PCR with nanopore measurement and data analysis, one can achieve accurate results fast. To that end, a single-control based quantification was developed that uses principal component analysis applied to the nanopore data (“S2 Text”). Only a single control mixture is required (nominally, the 50% Trait), which can be recorded during sub-5 min PCR of the test sample, followed by nanopore measurement of the PCR product, for a seed-to-answer result in less than 10 minutes. As all of the reagent components including the single polymerase enzyme are commercially available in bulk, are thermostable, can be lyophilized, and are not sensitive to light, production of assays would be uncomplicated, and storage and shipping of assays could be inexpensive at appreciable volumes.

A two-step extraction was tested, where the 35 g of seeds are first extracted using only water, and a small amount of that was then mixed with a NaOH or detergent containing solution, and then that small volume was diluted and/or neutralized before use a PCR template. These variations were fully compatible with the method, and reduced the amount of chemicals necessary, thus lowering the cost and environmental impact.

As described herein, the method is not limited to quantitative analysis of transgenes in mixtures of seed crops. Using the same PCR method, determination of zygosity of a transgene (or any chromosome rearrangement, natural or introduced) in individual organisms would be straightforward. Unlike traditional zygosity assays, which typically give a Y/N readout, the presented method could also be used to determine zygosity in polyploid organisms. It could also be used to quantify the frequency of a deletion, insertion, or rearrangement in a population of haploid organisms or organelles, such as bacteria, mitochondria, and chloroplasts.

### REFERENCES FROM EXAMPLE 3

1. Holst-Jensen A. Testing for genetically modified organisms (GMOs): Past, present and future perspectives. Biotechnology Advances. Elsevier Inc; 2009. Nov. 12; 27(6):1071-82.10.1016/j.biotechadv.2009.05.025
2. Nguyen H T, Jehle J A. Quantitative analysis of the seasonal and tissue-specific expression of Cry1Ab in transgenic maize Mon810. J Plant Dis Prot. Second edition. Springer Berlin Heidelberg; 2016. Mar. 31; 114(2):82-7.10.1007/BF03356208
3. Shrestha H K, Hwu K-K, Chang M-C. Advances in detection of genetically engineered crops by multiplex polymerase chain reaction methods. Trends in Food Science & Technology. 2010. September; 21(9):442-54.10.1016/j.tifs.2010.06.004
4. Chaouachi M, Berard A, Said K. Relative quantification in seed GMO analysis: state of art and bottlenecks. Transgenic Res. 2013. Feb. 12; 22(3):461-76.10.1007/s11248-012-9684-1
5. Huang C-C, Pan T-M. Event-Specific Real-Time Detection and Quantification of Genetically Modified Roundup Ready Soybean. J Agric Food Chem. 2005. May; 53(10):3833-9.10.1021/jf048580x
6. Gerdes L, Busch U, Pecoraro S. A statistical approach to quantification of genetically modified organisms (GMO) using frequency distributions. BMC Bioinformatics. BioMed Central; 2014. Dec. 14; 15(1):407 10.1016/j.bdq.2015.12.003
7. Kiddle G, Hardinge P, Buttigieg N, Gandelman O, Pereira C, McElgunn C J, et al. GMO detection using a bioluminescent real time reporter (BART) of loop mediated isothermal amplification (LAMP) suitable for field use. BMC Biotechnol. BioMed Central; 2012. Dec. 1; 12(1):1-13.10.1186/1472-6750-12-15
8. Auer C A. Tracking genes from seed to supermarket: techniques and trends. Trends Plant Sci. 2003. December; 8(12):591-7.10.1016/j.tplants.2003.10.010
9. Morin T J, McKenna W L, Shropshire T D, Wride D A, Deschamps J D, Liu X, et al. A handheld platform for target protein detection and quantification using disposable nanopore strips. Sci Rep. 2018. Oct.; 8(1):14834 10.1038/s41598-018-33086-7
10. Li J, Stein D, McMullan C, Branton D, Aziz M J, Golovchenko J A. Ion-beam sculpting at nanometre length scales. Nature. 2001. Jul. 12; 412(6843):166-9.10.1038/35084037
11. Storm A J, Chen J H, Zandbergen H W, Dekker C. Translocation of double-strand DNA through a silicon oxide nanopore. Phys Rev E. 2005. May; 71(5 Pt 1):051903 10.1103/PhysRevE.71.051903
12. Wanunu M, Sutin J, McNally B, Chow A, Meller A. DNA translocation governed by interactions with solid-state nanopores. Biophys J. 2008. Nov. 15; 95(10):4716-25.10.1529/biophysj.108.140475
13. Fologea D, Brandin E, Uplinger J, Branton D, Li J. DNA conformation and base number simultaneously determined in a nanopore. ELECTROPHORESIS. 2007. September; 28(18):3186-92.10.1002/elps.200700047
14. Squires A H, Atas E, Meller A. Genomic Pathogen Typing Using Solid-State Nanopores. Ahmed N, editor. PLoS ONE. 2015. Nov. 12; 10(11):e0142944-16.10.1371/journal.pone.0142944
15. Zhao Y, Mckenna W, Dunbar W B. Fractional abundance of polynucleotide sequences in a sample, International patent no. WO2018081178A1.2018.
16. Singer A, Wanunu M, Morrison W, Kuhn H, Frank-Kamenetskii M, Meller A. Nanopore based sequence specific detection of duplex DNA for genomic profiling. Nano Lett. 2010. Feb. 10; 10(2):738-42.10.1021/n1100058y
17. Morin T J, Shropshire T, Liu X, Briggs K, Huynh C, Tabard-Cossa V, et al. Nanopore-based target sequence detection. Wanunu M, editor. PLoS ONE. 2016. May 5; 11(5):e0154426-21.10.1371/journal.pone.0154426
18. Windels P, Taverniers I, Depicker A, Van Bockstaele E, De Loose M. Characterisation of the Roundup Ready soybean insert. Eur Food Res Technol. Springer-Verlag; 2001. Aug. 1; 213(2):107-12.10.1007/s002170100336

### OTHER EMBODIMENTS

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of quantifying a relative amount of genetic variants in a sample, comprising
a. mixing said sample with a set of primers capable of binding specifically to a target sequence to initiate an amplification reaction, said set of primers comprising
i. a first primer that binds specifically to a common sequence on a first strand of a first variant and a second variant in the sample, wherein said first primer is added at a reaction limiting concentration;
ii. a second primer that binds specifically to a second strand of said first variant; and
iii. a third primer that binds specifically to a second strand of said second variant;
b. performing an amplification reaction on said mixed sample to generate two amplification products of different length, wherein said first amplification product is generated from the first and second primer, and wherein the second amplification product is generated from the first and third primer;
c. detecting at least two distinct signals corresponding to the first amplification product and the second amplification product; and
d. quantifying the relative amount of the first and the second amplification products based on said detected signals.

2. The method of claim 1, wherein said amplification reaction is limited to align amplification rates of said first and second variants.

3. The method of any of the preceding claims, wherein at least one component of the amplification reaction is provided at a limiting reaction to align amplification rates of said first and second variants.

4. The method of claim 1, wherein said amplification reaction is inhibited by PCR conditions, a PCR blocking oligonucleotide, or sequence specific cleavage of the DNA template.

5. The method of claim 1, wherein said sample is derived from an organism or a population of organisms.

6. The method of any of the preceding claims, wherein said relative amount of genetic variants is used to determine a zygosity of said organism.

7. The method of any of the preceding claims, wherein said organism is suspected of being a genetically modified organism.

8. The method of any of the preceding claims, wherein at least one of said genetic variants is recombinantly engineered.

9. The method of any of the preceding claims, further comprising amplifying a control gene in said sample, and quantifying one or both of said amplification products relative to said amplified control gene.

10. The method of any of the preceding claims, wherein said quantification determines a zyogosity of an organism comprising said genetic variants.

11. The method of any of the preceding claims, wherein at least one of said genetic variants comprises a recombinantly engineered gene.

12. The method of any of the preceding claims, wherein at least one of said genetic variants comprise an inserted sequence.

13. The method of any of the preceding claims, wherein at least one of said genetic variants comprises a genetic rearrangement.

14. The method of any of the preceding claims, wherein said sample is derived from a virus, a protozoan, a fungus, a mold, a plant, an animal, or a human.

15. The method of any of the preceding claims, wherein said amplification reaction is selected from PCR or isothermal amplification.

16. The method of any of the preceding claims, wherein said distinct signal is detected using a nanopore device.

17. The method of any of the preceding claims, wherein said signals from said first and second genetic variants are discriminated by a characteristic selected from the group consisting of: amplicon length, sequence, physical or chemical modification incorporated into the primer, and physical or chemical probe added to the amplicon post-amplification.

18. The method of any of the preceding claims, wherein said physical or chemical probe comprises PEG.

19. The method of any of the preceding claims, wherein said physical or chemical probe comprises a fluorophore

20. The method of any of the preceding claims, wherein said PEG or fluorophore is bound to DNA, LNA, XNA, or PNA.

21. The method of any of the preceding claims, wherein said amplification reaction comprises one or more modified nucleotides or one or more modified primers.

22. The method of any of the preceding claims, wherein said modification comprises a direct label or an indirect label.

23. The method of any of the preceding claims, wherein said modification comprises a charged chemical moiety, a neutral chemical moiety, a hydrophobic moiety, or a hydrophilic moiety.

24. The method of any of the preceding claims, wherein said modification comprises a fluorescent dye.

25. The method of any of the preceding claims, wherein said detection is performed using a sensor configured to measures an electrical signal that fluctuates upon translocation of said first and/or second amplification product through a nanopore.

26. The method of any of the preceding claims, wherein said electrical signal is distinct between said first and second amplification products.

27. The method of any of the preceding claims, wherein the set of primers further comprises a fourth primer and a fifth primer that each each bind to a third strand and a fourth strand, wherein the third primer binds to the third strand.

28. The method of any of the preceding claims, wherein performing said amplification reaction on said mixed sample further generates a third amplification product and a fourth amplification product.

29. The method of any of the preceding claims, wherein said four amplification products are each of different lengths.

30. The method of any of the preceding claims, wherein said four amplification products are of three different lengths, with two amplification products being the same length.

31. The method of any of the preceding claims, wherein said third amplification product is generated from the fourth primer and the third primer, and said fourth amplification product is generated from the fourth primer and the fifth primer.

32. The method of any of the preceding claims, wherein said first or second variant comprises a single nucleotide polymorphism.

33. The method of any of the preceding claims, wherein said first or second variant comprises a silent mutation, a missense mutation, or a nonsense mutation.

34. The method of any of the preceding claims, wherein said first or second variant comprises a modified nucleotide or a non-natural nucleotide.

35. The method of any of the preceding claims, wherein the method further comprises, prior to detecting, loading the first amplification product onto a nanopore device.

36. The method of any of the preceding claims, wherein the method further comprises, prior to detecting, loading the second amplification product onto a nanopore device.

37. The method of any of the preceding claims, wherein the method further comprises applying a voltage at least one nanopore for translocating the first and/or second amplification product through the at least one nanopore.

38. The method of any of the preceding claims, wherein the first primer is a forward primer selected from TCAAACCCTTCAATTTAACCGA (SEQ ID NO:5); AACTACCTTCTCACCGCATTC (SEQ ID NO: 10); CGAGCTTCTTCACGAACTTCTC (SEQ ID NO: 11); ACCGCATTCGAGCTTCTT (SEQ ID NO: 12); CTTTCTGTTGGAAGAGAACTACCT (SEQ ID NO: 13); GAGAGATCTTCGCTGTGCAA (SEQ ID NO: 14); GCAATTGCGTGGTGAACT (SEQ ID NO: 15); AGGCCATTCGCCTCAAA (SEQ ID NO: 16); CACGAACTTCTCGACGATGG (SEQ ID NO: 17); GGCCATTCGCCTCAAACAG (SEQ ID NO: 18); and CCCTTCAATTTAACCGATGCTAAT (SEQ ID NO: 19).

39. The method of any of the preceding claims, wherein the second primer is a reverse primer selected from: CAGTTAACCAAACATGTCCTAAATC (SEQ ID NO: 3); GCCCATATCTAGGAAGCCAATAC (SEQ ID NO: 20); AAGAAGAGTACCTCGGAGAGAG (SEQ ID NO: 8); CCACACCTAAATGTCATAACTCATAAAC (SEQ ID NO: 21); AGATCGGGAGGGAAGAGATT (SEQ ID NO: 22); GTACAAGGAGGCGCCAAATA (SEQ ID NO: 23); TTCGTATTGTAATCTCCCTCAGAAT (SEQ ID NO: 24); TCCAAGTACTAGAGAAAGGCTTAAT (SEQ ID NO: 25); AGGAAGCCAATACAGTCGATATAA (SEQ ID NO: 26); TCACTGGCATACGAACAATTCA (SEQ ID NO: 27); TGGAGTCCAAGTACTAGAGAAAGG (SEQ ID NO: 28); TCCCTCAGAATTTCTTAATCTTGTG (SEQ ID NO: 29); GAACAGTTAACCAAACATGTCCTAA (SEQ ID NO: 30); and TTCGTATTGTAATCTCCCTCAGAA (SEQ ID NO: 31).

40. The method of any of the preceding claims, wherein the third primer is a reverse primer selected from CATCTTCAACGATGGCCTTTC (SEQ ID NO: 4); GGAGTTTCTCCTCCTGCTATTAC (SEQ ID NO: 32); CTCCCAGAATGATCGGAGTTTC (SEQ ID NO: 9); ACACTCACCAGTGACCCTAATA (SEQ ID NO: 33); TGATCGGAGTTTCTCCTCCT (SEQ ID NO: 34); GGTCATTTGTTGAAGATAGGAAACC (SEQ ID NO: 35); AAGGAGTAGTACACTCACCAGT (SEQ ID NO: 36); CCTAATAGGCAACAGCATGAAA (SEQ ID NO: 37); TCAACATGTGAAGGAGTAGTACA (SEQ ID NO: 38); GCATCTACATATAGCTTCTCGTTGT (SEQ ID NO: 39); GTACACTCACCAGTGACCCTAATA (SEQ ID NO: 40); CCCTAATAGGCAACAGCATGAA (SEQ ID NO: 41); and CAACGATGGCCTTTCCTTTATC (SEQ ID NO: 42).
